(19) United States
(12) Patent Application Publication
(10) Pub. No.: US 2020/0340041 A1
(43) Pub. Date: Oct. 29, 2020

(54) NOVEL COMPOSITIONS, METHODS AND KITS FOR URINARY TRACT MICROORGANISM DETECTION

(71) Applicant: LIFE TECHNOLOGIES CORPORATION, Carlsbad, CA (US)

(72) Inventors: Kelly LI, San Jose, CA (US); Ioanna PAGANI, Oakland, CA (US); Jisheng LI, Pleasanton, CA (US); Sunali PATEL, Austin, TX (US); Kamini VARMA, Saratoga, CA (US); Jorge FONSECA, San Francisco, CA (US); Nitin PURI, Pleasanton, CA (US); Evan DIAMOND, Austin, TX (US)

(21) Appl. No.: 16/763,583

(22) PCT Filed: Nov. 13, 2018
(86) PCT No.: PCT/US2018/060840

§ 371 Date: May 13, 2020

Related U.S. Application Data
(60) Provisional application No. 62/585,273, filed on Nov. 13, 2017.

(51) Int. Cl. C12Q 1/689; C12Q 1/686; C12Q 1/6837

(52) U.S. Cl. CPC C12Q 1/689; C12Q 2600/16; C12Q 1/6837; C12Q 1/686

(57) ABSTRACT

Various methods are disclosed for amplifying nucleic acid sequences in a nucleic acid sample. The methods involve forming at least five amplification reaction mixes each including an aliquot from a sample source that includes nucleic acid sequences, using at least five different assays each including a pair of amplification primers, the assays selected from the group of assays in Table 1 and/or targeting the sequences specified in Table 1.

### BACKGROUND

A wide variety of microorganisms can cause or contribute to diseases and disorders. Infectious agents can spread from individual to individual and lead to sickness in the population. Microorganisms which exist on or within a host in a symbiosis can lead to host diseases when imbalances arise in the microbial populations of an individual. The human microbiome project is providing rich insights into the composition of human and animal microbiomes and the ability to maintain balance in specific tissues.

Urogenital, bladder, and urinary tract tissues are rich environments where incidences of bacterial, fungal, viral, and/or parasitic microorganisms (e.g., uropathogens) can cause imbalance, leading to severe impact at the site.

Each year around 150 million people are affected by urinary tract infections (UTIs), which present serious health issues regardless of whether they are symptomatic or asymptomatic. Currently, UTIs are diagnosed based on clinical symptoms and urine analysis (bacteria culture, and presence of white blood cells) and treated with antibiotics. However, the human urinary tract hosts a diverse and complex microbial community, and emerging evidences show that bladder and urinary tract microbiota (UTM) may exert a profound effect on urologic health, both positive and negative. Current diagnostic methodologies for the urinary tract suffer from lack of target throughput, and rely on microorganism culture analysis (urinalysis). By contrast, panel-based molecular testing may not only identify the presence of a specific species, but also profile urinary microbiota, which would assist in understanding its biological significance and may potentially provide guidance of the proper antibiotics thereby reducing overtreatment.

The traditional culture-based method oftentimes misses pathogen bacteria or fungi detection in UTIs, especially in a polymicrobial or mixed flora environment. This is at least in part, because not all uropathogens grow equally well under standard culture conditions which can result in a failure to detect certain species and/or microbes. Additionally, the current culture-based method is time consuming, has low throughput, and can lack sensitivity and/or specificity. Thus, the polymicrobial nature of urinary tract infection requires the development of an assay system that can overcome the limitations inherent in urine culture and provide a rapid accurate measurement of the uropathogens present in the urine. Current technologies for use in urinary microbial monitoring and detection are costly, lack sensitivity and/or specificity, and/or require a complicated or lengthy workflow. There is a need for specific, efficient, and cost-effective systems for monitoring and profiling urogenital, bladder, and urinary tract infection and microbiota.

### SUMMARY

In one aspect, provided are methods for amplifying a plurality of nucleic acid sequences in a nucleic acid sample comprising: forming at least five amplification reaction mixes each comprising an aliquot from a sample source comprising a plurality of nucleic acid sequences, using at least five different assays each comprising a pair of amplification primers, the assays selected from the group of assays in Table 1; applying each amplification reaction mix to a reaction vessel; performing a plurality of amplification reactions on the reaction vessel; and detecting an amplification product corresponding to a target nucleic acid sequence within one or more locations on the reaction vessel during the plurality of amplification reactions. In one embodiment the method further comprises: utilizing the reaction in an amplification product detection system; and operating the amplification product detection system to: associate locations of the amplification reaction mix on the reaction vessel with one or more of the assay IDs utilized in the amplification reaction mix, optionally by use of an association table. In one embodiment of the method, the reaction vessel is a plate with a plurality of wells. In another embodiment of the method, the reaction vessel is an array. In another embodiment of the method, the reaction vessel is an open array plate. In yet another embodiment of the method, the reaction vessel is a chip microarray. In one embodiment, the method comprises forming at least ten amplification reaction mixes each comprising an aliquot from a sample source comprising a plurality of nucleic acid sequences, using at least ten different assays selected from the group of assays in Table 1. In one embodiment, the method comprises forming at least fifteen amplification reaction mixes each comprising an aliquot from a sample source comprising a plurality of nucleic acid sequences, using at least fifteen different assays selected from the group of assays in Table 1. In one embodiment, the method comprises forming seventeen amplification reaction mixes each comprising an aliquot from a sample source comprising a plurality of nucleic acid sequences, using seventeen different assays selected from the group of assays in Table 1. In one embodiment, the method comprises forming reaction mixes each comprising an aliquot from a sample source comprising a plurality of nucleic acid sequences, using all of the assays in Table 1. In one embodiment of the method, the sample source is a urine specimen. In one embodiment of the method, the amplification product comprises a target amplicon of the nucleic acid sample having an amplicon length of between 56 to 105 nucleotides. In one embodiment of the method, the assay ID Ba04932084_s1 comprises a pair of primers targeting a portion of a nucleic acid sequence of an unannotated region of a gene of Acinetobacter baumannii. In one embodiment of the method, the assay ID Ba04932088_s1 comprises a pair of primers that targets a portion of a nucleic acid sequence of an oxalate decarboxylase/archaeal phosphoglucose isomerase, cupin superfamily gene of Citrobacter freundii, such as, for example, COG2140. In one embodiment of the method, the assay ID Ba07286617_s1 and/or Ba07286616_s1 comprises a pair of primers that targets a portion of a nucleic acid sequence for an iron complex transport system substrate-binding protein of Citrobacter freundii. In one embodiment of the method, the assay ID Ba04932080_s1 comprises a pair of primers that targets a portion of a nucleic acid sequence of a pyridoxal phosphate-dependent histidine decarboxylase (hdc) gene of Klebsiella aerogenes (previously known as Enterobacter aerogenes). In one embodiment of the method, the assay ID Ba04932087_s1 comprises a pair of primers that targets a portion of a nucleic acid sequence of a hypothetical protein of a gene of Enterobacter cloacae. In one embodiment of the method, the assay ID Ba04646247_s1 comprises a pair of primers that targets a portion of a nucleic acid sequence of an aminotransferase class V gene of Enterococcus faecalis. In one embodiment of the method, the assay ID Ba04932086_s1 comprises a pair of primers that targets a portion of a nucleic acid sequence of a PhnB -MerR family transcriptional regulator gene of Enterococcus faecium. In one embodiment of the method, the assay ID Ba04646242_s1 comprises a pair of primers that targets a portion of a nucleic acid sequence of a DNA-binding transcriptional regulator MerR family (Zntr) gene of Escherichia coli, such as, for example, COG0789. In one embodiment of the method, the assay ID Ba04932079_s1 comprises a pair of primers that targets a portion of a nucleic acid sequence of a parC (DNA topoisomerase IV subunit A) gene of Klebsiella oxytoca. In one embodiment of the method, the assay ID Ba04932083_s1 comprises a pair of primers that targets a portion of a nucleic acid sequence an act-like protein gene of Klebsiella pneumoniae. In one embodiment of the method, the assay ID Ba04932078_s1 comprises a pair of primers that targets a portion of a nucleic acid sequence of a COG1918 for Fe2+ transport system protein FeoA gene of Morganella morganii. In one embodiment of the method, the assay ID Ba04932076_s1 comprises a pair of primers that targets a portion of a nucleic acid sequence of an araC, ureR gene of Proteus mirabilis. In one embodiment of the method, the assay ID Ba04932077_s1 comprises a pair of primers that targets a portion of a nucleic acid sequence of a SUMF1 gene of Proteus vulgaris. In one embodiment of the method, the assay ID Ba04932082_s1 comprises a pair of primers that targets a portion of a nucleic acid sequence of a Sulfatase maturation enzyme As1B, radical SAM superfamily, putative iron-sulfur modifier protein gene of Providencia stuartii, such as, for example, COG0641. In one embodiment of the method, the assay ID Ba04932081_s1 comprises a pair of primers that targets a portion of a nucleic acid sequence of a N296_1760, helix turn helix domain protein gene of Pseudomonas aeruginosa. In one embodiment of the method, the assay ID Ba04932085_s1 comprises a pair of primers that targets a portion of a nucleic acid sequence of a cdaR gene of Staphylococcus saprophyticus. In one embodiment of the method, the assay ID Ba04646276_s1 comprises to a pair of primers that targets a portion of a nucleic acid sequence of a SIP gene of Streptococcus agalactiae. In one embodiment of the method, the assay ID Fn04646233_s1 comprises a pair of primers that targets a portion of a nucleic acid sequence of an IPT1 gene of Candida albicans.

In another aspect, provided is a method for amplifying a plurality of nucleic acid sequences in a nucleic acid sample comprising: forming a plurality of amplification reaction mixes each comprising an aliquot from a sample source comprising a plurality of nucleic acid sequences, wherein the sample source is a urine specimen; applying the plurality of amplification reaction mixes to a reaction vessel, where the reaction vessel is configured with at least five assays each targeting a different gene having the corresponding target region positions and the corresponding target region sizes in Table 1, and wherein each an assay comprises a pair of amplification primers; performing a plurality of amplification reactions on the reaction vessel; and detecting an amplification product corresponding to a target nucleic acid sequence within locations on the reaction vessel during the plurality of amplification reactions. In one embodiment, the method further comprises: utilizing the reaction vessel in an amplification product detection system; and operating the amplification product detection system to: associate locations of the amplification reaction mix on the reaction vessel with one or more of the assay utilized on the reaction vessel, optionally by use of an association table. In one embodiment of the method, the reaction vessel is a plate with a plurality of wells. In another embodiment of the method, the reaction vessel is an array. In another embodiment of the method, the reaction vessel is an open array plate. In yet another embodiment of the method, the reaction vessel is a chip microarray. In one embodiment of the method, the reaction vessel is configured with at least ten assays each targeting a different gene listed in Table 1. In one embodiment of the method, the reaction vessel is configured with at least fifteen assays each targeting a different gene listed in Table 1. In one embodiment of the method, the reaction vessel is configured with assays targeting seventeen of the genes listed in Table 1. In one embodiment of the method, the reaction vessel is configured with assays targeting each of the genes listed in Table 1. In one embodiment, the method comprises an assay, of the at least five assays, that amplifies an amplicon that is 93 nucleotides long and corresponds to a gene for an unannotated region in Acinetobacter baumannii. In one embodiment, the method comprises an assay, of the at least five assays, that amplifies an amplicon that is 103 nucleotides long and corresponds to an oxalate decarboxylase/archaeal phosphoglucose isomerase, cupin superfamily gene in Citrobacter freundii, including, for example, COG2140. In one embodiment, the method comprises an assay, of the at least five assays, that amplifies an amplicon that is 62 and/or 110 nucleotides long and corresponds to a portion of a nucleic acid sequence for an iron complex transport system substrate-binding protein of Citrobacter freundii. In one embodiment, the method comprises an assay, of the at least five assays, that amplifies an amplicon that is 98 nucleotides long and corresponds to a pyridoxal phosphate-dependent histidine decarboxylase (hdc) gene in Klebsiella aerogenes (previously known as Enterobacter aerogenes). In one embodiment, the method comprises an assay, of the at least five assays, that amplifies an amplicon that is 88 nucleotides long and corresponds to a gene for a hypothetical protein in Enterobacter cloacae. In one embodiment, the method comprises an assay, of the at least five assays, that amplifies an amplicon that is 95 nucleotides long and corresponds to aminotransferase class V gene in Enterococcus faecalis. In one embodiment, the method comprises an assay, of the at least five assays, that amplifies an amplicon that is 98 nucleotides long and corresponds to a PhnB -MerR family transcriptional regulator gene in Enterococcus faecium. In one embodiment, the method comprises an assay, of the at least five assays, that amplifies an amplicon that is 63 nucleotides long and corresponds to DNA-binding transcriptional regulator MerR family (Zntr) gene in Escherichia coli, including, for example, COG0789. In one embodiment, the method comprises an assay, of the at least five assays, that amplifies an amplicon that is 93 nucleotides long and corresponds to a parC (DNA topoisomerase IV subunit A) gene in Klebsiella oxytoca. In one embodiment, the method comprises an assay, of the at least five assays, that amplifies an amplicon that is 56 nucleotides long and corresponds to and act-like protein in Klebsiella pneumoniae. In one embodiment, the method comprises an assay, of the at least five assays, that amplifies an amplicon that is 91 nucleotides long and corresponds to a Fe2+ transport system protein FeoA gene in Morganella morganii, including, for example, COG1918. In one embodiment, the method comprises an assay, of the at least five assays, that amplifies an amplicon that is 100 nucleotides long and corresponds to an araC, ureR gene in Proteus mirabilis. In one embodiment, the method comprises an assay, of the at least five assays, that amplifies an amplicon that is 76 nucleotides long and corresponds to a SUMF1 gene in Proteus vulgaris. In one embodiment, the method comprises an assay, of the at least five assays, that amplifies an amplicon that is 100 nucleotides long and corresponds to a gene for a Sulfatase maturation enzyme As1B, radical SAM superfamily, putative iron-sulfur modifier protein in Providencia stuartii, including, for example, COG0641. In one embodiment, the method comprises an assay, of the at least five assays, that amplifies an amplicon that is 70 nucleotides long a gene for a helix turn helix domain protein in Pseudomonas aeruginosa, including, for example, N296_1760. In one embodiment, the method comprises an assay, of the at least five assays, that amplifies an amplicon that is 85 nucleotides long and corresponds to a cdaR gene in Staphylococcus saprophyticus. In one embodiment, the method comprises an assay, of the at least five assays, that amplifies an amplicon that is 66 nucleotides long and corresponds to a SIP gene in Streptococcus agalactiae. In one embodiment, the method comprises an assay, of the at least five assays, that amplifies an amplicon that is 105 nucleotides long and corresponds to an IPT1 gene in Candida albicans.

In another aspect, provided is a composition for determining the presence or absence of at least one target nucleic acid in a biological sample, the composition comprising: at least five different amplification primer pairs, wherein each of said primers of said pairs comprise a target hybridization region that is configured to specifically hybridize to all or a portion of a region of a nucleic acid sequence of a target microorganism in Table 1 and wherein under suitable conditions said primer pair generates an amplicon; and at least five detection probes configured to specifically hybridize to all or a portion of a region of said amplicon produced by said primer pairs. In one embodiment, the composition further comprises a control nucleic acid molecule comprising a plurality of different nucleic acid target sequences, said plurality of different nucleic acid target sequences being specific to at least five genes in Table 1. In one embodiment, the composition is a panel or a collection of assays. In one embodiment, the panel or collection of assays comprise a panel or collection of TaqMan Assays. In one embodiment of the composition, the at least one target nucleic acid is a biomarker for a microbe associated with a urinary tract infection. In one embodiment, the composition comprises a solid support. In one embodiment of the composition, the at least five amplification primer pairs are separated by location on the solid support. In one embodiment, the composition comprises at least ten amplification different primer pairs, wherein each of said primers of said pair comprises a target hybridization region that is configured to specifically hybridize to all or a portion of a region of a nucleic acid sequence of target microorganisms in Table 1 and wherein under suitable conditions said primer pair generates an amplicon. In one embodiment, the composition comprises at least fifteen different amplification primer pairs, wherein each of said primers of said pair comprises a target hybridization region that is configured to specifically hybridize to all or a portion of a region of a nucleic acid sequence of target microorganisms in Table 1 and wherein under suitable conditions said primer pair generates an amplicon. In one embodiment, the composition comprises at least seventeen different amplification primer pairs, wherein each of said primers of said pair comprises a target hybridization region that is configured to specifically hybridize to all or a portion of a region of a nucleic acid sequence of target microorganisms in Table 1 and wherein under suitable conditions said primer pair generates an amplicon. In one embodiment of the composition, the at least one target nucleic acid is specific for Acinetobacter baumannii and is within a 701 nucleic acid sequence in accession number NZ_GG704574.1 positioned in a region corresponding to nucleotides 202100-202800 of the Acinetobacter baumannii genome. In one embodiment of the composition, the at least one target nucleic acid is specific for Citrobacter freundii and is within a 801 nucleic acid sequence in accession number NZ_ANAV01000004.1 positioned in a region corresponding to nucleotides 137400-138200 of the Citrobacter freundii genome. In one embodiment of the composition, the at least one target nucleic acid is specific for Citrobacter freundii and is within a 801 nucleic acid sequence in accession number NZ_ANAV01000001.1 positioned in a region corresponding to nucleotides 277000-277800 of the Citrobacter freundii genome. In one embodiment of the composition, the at least one target nucleic acid is specific for Klebsiella aerogenes (previously known as Enterobacter aerogenes) and is within a 801 nucleic acid sequence in accession number CP014748.1 positioned in a region corresponding to nucleotides 1158600-1159400 of the Klebsiella aerogenes (previously known as Enterobacter aerogenes) genome. In one embodiment of the composition, the at least one target nucleic acid is specific for Enterobacter cloacae and is within a 801 nucleic acid sequence in accession number CP008823.1 positioned in a region corresponding to nucleotides 3274000-3274800 of the Enterobacter cloacae genome. In one embodiment of the composition, the at least one target nucleic acid is specific for Enterococcus faecalis and is within a 801 nucleic acid sequence in accession number HF558530.1 positioned in a region corresponding to nucleotides 1769100-1769900 of the Enterococcus faecalis genome. In one embodiment of the composition, the at least one target nucleic acid is specific for Enterococcus faecium and is within a 801 nucleic acid sequence in accession number NZ_GL476131.1 positioned in a region corresponding to nucleotides 17300-18100 of the Enterococcus faecium genome. In one embodiment of the composition, the at least one target nucleic acid is specific for Escherichia coli and is within a 701 nucleic acid sequence in accession number CP015843.2 positioned in a region corresponding to nucleotides 4336000-4336700 of the Escherichia coli genome. In one embodiment of the composition, the at least one target nucleic acid is specific for Klebsiella oxytoca and is within a 801 nucleic acid sequence in accession number CP020358.1 positioned in a region corresponding to nucleotides 2851700-2852600 of the Klebsiella oxytoca genome. In one embodiment of the composition, the at least one target nucleic acid is specific for Klebsiella pneumoniae and is within a 801 nucleic acid sequence in accession number CP007727.1 positioned in a region corresponding to nucleotides 209000-2090800 of the Klebsiella pneumoniae genome. In one embodiment of the composition, the at least one target nucleic acid is specific for Morganella morganii and is within a 801 nucleic acid sequence in accession number CP004345.1 positioned in a region corresponding to nucleotides 375800-376600 of the Morganella morganii genome. In one embodiment of the composition, the at least one target nucleic acid is specific for Proteus mirabilis and is within a 801 nucleic acid sequence in accession number CP017082.1 positioned in a region corresponding to nucleotides 580200-581000 of the Proteus mirabilis genome. In one embodiment, the composition further comprises a polymerase having 5′ nuclease activity. In some embodiments, the polymerase is thermostable. In some embodiments, the polymerase is Taq DNA polymerase. In one embodiment, the detection probes of the composition are TaqMan probes or 5′nuclease probes.

In one embodiment of the composition, the at least one target nucleic acid is specific for Proteus vulgaris and is within a 801 nucleic acid sequence in accession number JPIX01000006.1 positioned in a region corresponding to nucleotides 10200-102800 of the Proteus vulgaris genome. In one embodiment of the composition, the at least one target nucleic acid is specific for Providencia stuartii and is within a 801 nucleic acid sequence in accession number NZ_DS607663.1 positioned in a region corresponding to nucleotides 493000-493800 of the Providencia stuartii genome. In one embodiment of the composition, the at least one target nucleic acid is specific for Pseudomonas aeruginosa and is within a 801 nucleic acid sequence in accession number CP006831.1 positioned in a region corresponding to nucleotides 1857600-1858400 of the Pseudomonas aeruginosa genome. In one embodiment of the composition, the at least one target nucleic acid is specific for Staphylococcus saprophyticus and is within a 601 nucleic acid sequence in accession number AP008934.1 positioned in a region corresponding to nucleotides 200400-201000 of the Staphylococcus saprophyticus genome. In one embodiment of the composition, the at least one target nucleic acid is specific for Streptococcus agalactiae and is within a 601 nucleic acid sequence in accession number CP010319.1 positioned in a region corresponding to nucleotides 41000-41600 of the Streptococcus agalactiae genome. In one embodiment of the composition, the at least one target nucleic acid is specific for Candida albicans and is within a 701 nucleic acid sequence in accession number AY884203.1 positioned in a region corresponding to nucleotides 800-1500 of the Candida albicans genome.

In another aspect, provided is a nucleic acid construct for evaluating a plurality of amplification reactions, the nucleic acid construct comprising: a control nucleic acid molecule comprising a plurality of different nucleic acid target sequences, said plurality of target nucleic acid sequences directed to at least five genes in Table 1 inserted into a DNA plasmid. In one embodiment of the nucleic acid construct, said plurality of target nucleic acid sequences directed to at least ten of the genes in Table 1 in the DNA plasmid. In one embodiment of the nucleic acid construct, said plurality of target nucleic acid sequences directed to at least fifteen of the genes in Table 1 in the DNA plasmid. In one embodiment of the nucleic acid construct, said plurality of target nucleic acid sequences directed to each of the genes in Table 1 in the DNA plasmid. In one embodiment of the nucleic acid construct, a target nucleic acid sequence for Acinetobacter baumannii is within a 701 nucleic acid sequence in accession number NZ_GG704574.1 positioned in a region corresponding to nucleotides 202100-202800 of the Acinetobacter baumannii genome. In one embodiment of the nucleic acid construct, a target nucleic acid sequence for Citrobacter freundii is within a 801 nucleic acid sequence in accession number NZ_ANAV01000004.1 positioned in a region corresponding to nucleotides 137400-138200 of the Citrobacter freundii genome. In one embodiment of the nucleic acid construct, a target nucleic acid sequence for Citrobacter freundii is within a 801 nucleic acid sequence in accession number NZ_ANAV01000001.1 positioned in a region corresponding to nucleotides 277000-277800 of the Citrobacter freundii genome. In one embodiment of the nucleic acid construct, a target nucleic acid sequence for Klebsiella aerogenes (previously known as Enterobacter aerogenes) is within a 801 nucleic acid sequence in accession number CP014748.1 positioned in a region corresponding to nucleotides 1158600-1159400 of the Klebsiella aerogenes (previously known as Enterobacter aerogenes) genome. In one embodiment of the nucleic acid construct, a target nucleic acid sequence for Enterobacter cloacae is within a 801 nucleic acid sequence in accession number CP008823.1 positioned in a region corresponding to nucleotides 3274000-3274800 of the Enterobacter cloacae genome. In one embodiment of the nucleic acid construct, a target nucleic acid sequence for Enterococcus faecalis is within a 801 nucleic acid sequence in accession number HF558530.1 positioned in a region corresponding to nucleotides 1769100-1769900 of the Enterococcus faecalis genome. In one embodiment of the nucleic acid construct, a target nucleic acid sequence for Enterococcus faecium is within a 801 nucleic acid sequence in accession number NZ_GL476131.1 positioned in a region corresponding to nucleotides 17300-18100 of the Enterococcus faecium genome. In one embodiment of the nucleic acid construct, a target nucleic acid sequence for Escherichia coli is within a 701 nucleic acid sequence in accession number CP015843.2 positioned in a region corresponding to nucleotides 4336000-4336700 of the Escherichia coli genome. In one embodiment of the nucleic acid construct, a target nucleic acid sequence for Klebsiella oxytoca is within a 801 nucleic acid sequence in accession number CP020358.1 positioned in a region corresponding to nucleotides 2851700-2852600 of the Klebsiella oxytoca genome. In one embodiment of the nucleic acid construct, a target nucleic acid sequence for Klebsiella pneumoniae is within a 801 nucleic acid sequence in accession number CP007727.1 positioned in a region corresponding to nucleotides 209000-2090800 of the Klebsiella pneumoniae genome. In one embodiment of the nucleic acid construct, a target nucleic acid sequence for Morganella morganii is within a 801 nucleic acid sequence in accession number CP004345.1 positioned in a region corresponding to nucleotides 375800-376600 of the Morganella morganii genome. In one embodiment of the nucleic acid construct, a target nucleic acid sequence for Proteus mirabilis is within a 801 nucleic acid sequence in accession number CP017082.1 positioned in a region corresponding to nucleotides 580200-581000 of the Proteus mirabilis genome. In one embodiment of the nucleic acid construct, a target nucleic acid sequence for Proteus vulgaris is within a 801 nucleic acid sequence in accession number JPIX01000006.1 positioned in a region corresponding to nucleotides 10200-102800 of the Proteus vulgaris genome. In one embodiment of the nucleic acid construct, a target nucleic acid sequence for Providencia stuartii is within a 801 nucleic acid sequence in accession number NZ_DS607663.1 positioned in a region corresponding to nucleotides 493000-493800 of the Providencia stuartii genome. In one embodiment of the nucleic acid construct, a target nucleic acid sequence for Pseudomonas aeruginosa is within a 801 nucleic acid sequence in accession number CP006831.1 positioned in a region corresponding to nucleotides 1857600-1858400 of the Pseudomonas aeruginosa genome. In one embodiment of the nucleic acid construct, a target nucleic acid sequence for Staphylococcus saprophyticus is within a 601 nucleic acid sequence in accession number AP008934.1 positioned in a region corresponding to nucleotides 200400-201000 of the Staphylococcus saprophyticus genome. In one embodiment of the nucleic acid construct, a target nucleic acid sequence for Streptococcus agalactiae is within a 601 nucleic acid sequence in accession number CP010319.1 positioned in a region corresponding to nucleotides 41000-41600 of the Streptococcus agalactiae genome. In one embodiment of the nucleic acid construct, a target nucleic acid sequence for Candida albicans is within a 701 nucleic acid sequence in accession number AY884203.1 positioned in a region corresponding to nucleotides 800-1500 of the Candida albicans genome.

In another aspect, provided is a method for amplifying a plurality of nucleic acid sequences in a nucleic acid sample, comprising: performing a plurality of amplification reactions, said amplification reactions each comprising a portion of a nucleic acid sample and a pair of amplification primers each configured to produce an amplification product corresponding to a different target nucleic acid sequence from a group of target nucleic acid sequences associated with the organisms and corresponding amplicon sizes, regions, and accession numbers set forth in Table 1; forming a plurality of different amplification products from the amplification reactions; and determining the presence or absence of at least one of said plurality of different amplification products. In one embodiment, the method comprises performing the plurality of amplification reactions, wherein at least ten of the amplification reactions contain a portion of a nucleic acid sample and a pair of amplification primers each configured to produce an amplification product corresponding to a different target nucleic acid sequence from the group of target nucleic acid sequences associated with the organisms and corresponding amplicon sizes, regions, and accession numbers set forth in Table 1. In one embodiment, the method comprises performing the plurality of amplification reactions, where at least fifteen of the amplification reactions contain a portion of a nucleic acid sample and a pair of amplification primers each configured to produce an amplification product corresponding to a different target nucleic acid sequence from the group of target nucleic acid sequences associated with the organisms and corresponding amplicon sizes, regions, and accession numbers set forth in Table 1. In one embodiment, the method comprises performing the plurality of amplification reactions, where all of the amplification reactions, excluding a negative control, contain a portion of a nucleic acid sample and a pair of amplification primers, each configured to produce an amplification product corresponding to a different target nucleic acid sequence from the group of target nucleic acid sequences associated with the organisms and corresponding amplicon sizes, regions, and accession numbers set forth in Table 1.

In another aspect, provided is a method for amplifying a plurality of nucleic acid sequences in a nucleic acid sample, comprising: (a) performing a plurality of amplification reactions, at least five of said amplification reactions comprising a portion of a nucleic acid sample and a pair of amplification primers configured to produce an amplification product corresponding to said target nucleic acid sequence, wherein each target nucleic acid sequence is the amplification product of a different gene selected from the group of genes in Table 1; (b) forming a plurality of different amplification products; and (c) determining the presence or absence of at least one of said plurality of different amplification products. In one embodiment of the method at least five of said amplification reactions comprise a pair of amplification primers selected from an assay ID listed in Table 1. In one embodiment of the method, at least ten of said amplification reactions comprise a pair of amplification primers selected from an assay ID listed in Table 1. In one embodiment of the method, at least fifteen of said amplification reactions comprise a pair of amplification primers selected from an assay ID listed in Table 1. In one embodiment of the method, all of said amplification reactions comprise a pair of amplification primers selected from an assay ID listed in Table 1. The method comprising, in some embodiments, performing a plurality of amplification reactions, at least ten of said amplification reactions containing a portion of a nucleic acid sample and a pair of amplification primers configured to produce an amplification product corresponding to said target nucleic acid sequence, wherein said target nucleic acid sequence is the amplification product of a portion of the a gene listed in Table 1. The method comprising, in some embodiments, performing a plurality of amplification reactions, at least fifteen of said amplification reactions containing a portion of a nucleic acid sample and a pair of amplification primers configured to produce an amplification product corresponding to said target nucleic acid sequence, wherein each said target nucleic acid sequence is the amplification product of a different gene set forth in Table 1. The method comprising, in some embodiments, performing the plurality of amplification reactions, all of said amplification reactions, excluding a negative control, containing a portion of a nucleic acid sample and a pair of amplification primers configured to produce an amplification product corresponding to said target nucleic acid sequence, wherein each said target nucleic acid sequence is the amplification product of a different gene set forth in Table 1. In some embodiments of the method, said amplification product is between 56 to 105 nucleotides long. In some embodiments of the method, at least one pair of said amplification primers configured to produce an amplification product includes primers containing a nucleic acid sequence that is complementary or identical to a portion of said corresponding target nucleic acid sequence. In some embodiments of the method, said corresponding target nucleic acid sequence for at least one pair of said amplification primers contains a nucleic acid sequence that is identical or complementary to a nucleic acid sequence present in genomic DNA, RNA, miRNA, mRNA, cell-free DNA, circulating DNA or cDNA. In some embodiments of the method, said corresponding target nucleic acid sequence is present within or is derived from genomic DNA, RNA, miRNA, mRNA, cell-free DNA, circulating DNA or cDNA of a target microorganism. In some embodiments of the method, said target microorganism is a microorganism listed in Table 1. In some embodiments of the method, said forming includes forming in parallel between 10 and 10, 000 different amplification products. In some embodiments of the method, at least two of said plurality of amplification reactions each contains a pair of amplification primers configured to amplify a different corresponding target nucleic acid sequence. In some embodiments of the method, said corresponding target nucleic acid sequence contains a portion of a nucleic acid sequence of a gene listed in Table 1 or its corresponding cDNA. In some embodiments of the method, said gene is present within a microorganism listed in Table 1. In some embodiments of the method, each of said plurality of amplification reactions contains a set of amplification primers configured to produce an amplification product that is between 56 to 105 nucleotides long. In some embodiments of the method, said forming includes forming one or more amplification products containing a nucleic acid sequence that is complementary or identical to a portion of a gene listed in Table 1. In some embodiments of the method, said forming includes forming a separate amplification product for all of the genes listed in Table 1 using a nucleic acid sample derived from a microorganism listed in Table 1. In some embodiments of the method, said forming includes forming a separate amplification product for all the microorganism genes listed in Table 1.

In some embodiments of the method for amplifying a plurality of nucleic acid sequences in a nucleic acid sample, said forming includes forming a separate amplification product for any combination of at least two of the microorganism genes listed in Table 1. In some embodiments of the method, one or more of said plurality of amplification reactions further contains a detectably labeled probe that includes a sequence that is identical or complementary to a portion of said corresponding target nucleic acid sequence. In some embodiments of the method, said detectably labeled probe of at least one amplification reaction is configured to undergo cleavage by a polymerase having 5′ exonuclease activity. In some embodiments of the method, said detectably labeled probe of at least one amplification reaction contains a fluorescent label at its 5′ end and a quencher at its 3′ end. In some embodiments of the method, said detectably labeled probe further contains a minor groove binder (MGB) moiety. In some embodiments of the method, at least one of said amplification reactions occurs at an individual reaction site present within or upon a support, said support containing one or more individual reaction sites. In some embodiments of the method, said support is selected from a multi-well plate, a microfluidic card, and a plate comprising a plurality of through-hole reaction sites. In some embodiments of the method, said individual reaction site includes one or more of said amplification primers, and said amplifying further includes distributing a portion of said nucleic acid sample to said individual reaction site. In some embodiments of the method, said individual reaction site includes a dried deposit of a solution containing a pair of amplification primers and a nucleic acid probe, wherein said primers and probe are both configured to amplify a nucleic acid sequence derived from a gene listed in Table 1. In some embodiments of the method, said individual reaction site further includes a polymerase and/or nucleotides, distributed to said reaction site either prior to or after said portion of said nucleic acid sample is distributed to said reaction site. In some embodiments of the method, said nucleic acid sample is prepared from a urine specimen. In some embodiments, the method further comprises preparing said nucleic acid sample from a urine specimen prior to said performing said plurality of amplification reactions.

In another aspect, provided is a method for detecting the presence of a microorganism nucleic acid in a sample, said method comprising: (a) distributing portions of a nucleic acid sample to individual reaction chambers situated within a support; (b) performing parallel amplification reactions and forming at least five amplification products, each in individual reaction chambers, wherein each amplification reaction contains a pair of amplification primers configured to produce an amplification product corresponding to a target nucleic acid sequence present within, or derived from, the genome of a microorganism, wherein said corresponding target nucleic acid sequence contains a portion of the nucleic acid sequence of a gene listed in Table 1 or its corresponding cDNA; and (c) determining whether said amplification product has been formed in one or more of said individual reaction chambers. In one embodiment of the method, at least five of said amplification reactions comprise a pair of amplification primers selected from an assay ID listed in Table 1. In one embodiment of the method, at least ten of said amplification reactions comprise a pair of amplification primers selected from an assay ID listed in Table 1. In one embodiment of the method, at least fifteen of said amplification reactions comprise a pair of amplification primers selected from an assay ID listed in Table 1. In one embodiment of the method, all of said amplification reactions comprise a pair of amplification primers selected from an assay ID listed in Table 1. In one embodiment of the method, at least ten amplification products are formed during the parallel amplification reactions. In one embodiment of the method, at least fifteen amplification products are formed during the parallel amplification reactions. In one embodiment of the method, at least seventeen amplification products are formed during the parallel amplification reactions. In one embodiment of the method, said amplification product is between 56 to 105 nucleotides long. In one embodiment of the method, said determining includes detecting hybridization of a detectably labeled probe to said amplification product, optionally in real-time. In one embodiment of the method, at least one pair of said amplification primers configured to produce an amplification product corresponding to said target nucleic acid sequence includes primers containing a nucleic acid sequence that is complementary or identical to a portion of said corresponding target nucleic acid sequence. In one embodiment of the method, said corresponding target nucleic acid sequence for at least one pair of said amplification primers contains a nucleic acid sequence that is identical or complementary to a nucleic acid sequence present in genomic DNA, RNA, miRNA, mRNA, cell-free DNA, circulating DNA or cDNA. In one embodiment of the method, said corresponding target nucleic acid sequence is present within or is derived from genomic DNA, RNA, miRNA, mRNA, cell-free DNA, circulating DNA or cDNA of a target microorganism. In one embodiment of the method, said microorganism is a microorganism listed in Table 1. In one embodiment of the method, said forming includes forming in parallel between 10 and 10, 000 different amplification products.

In one embodiment of the method for detecting the presence of a microorganism nucleic acid in a sample, at least two of said amplification reactions each contains a pair of amplification primers configured to amplify a different corresponding target nucleic acid sequence. In one embodiment of the method, said gene is present within a microorganism listed in Table 1. In one embodiment of the method, each of said amplification reactions contains amplification primers configured to amplify at least a portion of a gene listed in Table 1. In one embodiment of the method, said forming includes forming one or more amplification products containing a nucleic acid sequence that is complementary or identical to a portion of a gene listed in Table 1. In one embodiment of the method, each of said plurality of amplification reactions contains a set of amplification primers configured to produce an amplification product that is between 56 to 105 nucleotides long. In one embodiment of the method, said forming includes forming a separate amplification product for all of the genes listed in Table 1 using a nucleic acid sample derived from a microorganism listed in Table 1. In one embodiment of the method, said forming includes forming a separate amplification product for all the microorganism genes listed in Table 1. In one embodiment of the method, said forming includes forming a separate amplification product for any combination of at least two of the microorganism genes listed in Table 1. In one embodiment of the method, one or more of said plurality of said amplification reactions further contains a detectably labeled probe that includes a sequence that is identical or complementary to a portion of the corresponding target nucleic acid sequence. In one embodiment of the method, said detectably labeled probe of at least one amplification reaction is configured to undergo cleavage by a polymerase having 5′ exonuclease activity. In one embodiment of the method, said detectably labeled probe of at least one amplification reaction contains a fluorescent label at its 5′ end and a quencher at its 3′ end. In one embodiment of the method, said detectably labeled probe further contains a minor groove binder (MGB) moiety. In one embodiment of the method, at least one of said amplification reactions occurs at an individual reaction site present within or upon a support, said support containing one or more individual reaction sites. In one embodiment of the method, said support is selected from a multi-well plate, a microfluidic card, and a plate comprising a plurality of through-hole reaction sites. In one embodiment of the method, said individual reaction site includes one or more of said amplification primers, and said amplifying further includes distributing a portion of the nucleic acid sample to said individual reaction site. In one embodiment of the method, said individual reaction chambers include a dried deposit of a solution containing a pair of amplification primers and a nucleic acid probe, wherein said primers and probe are both configured to amplify a nucleic acid sequence derived from a gene listed in Table 1. In one embodiment of the method, said individual reaction chambers further include a polymerase and/or nucleotides, distributed to the individual reaction chamber either prior to or after said portion of said nucleic acid sample is distributed to said reaction site. In one embodiment of the method, said nucleic acid sample is prepared from a urine specimen. In one embodiment, the method further comprises preparing said nucleic acid sample from a urine specimen prior to said distributing.

In another aspect, provided is a support for nucleic acid amplification, comprising: a support containing a plurality of reaction sites located within said support or on said support's surface; and at least five of said reaction sites containing: (1) an amplification primer pair configured to produce an amplification product corresponding target nucleic acid sequence, wherein said amplification product corresponds to a microorganism in Table 1, and (2) a detectably labeled probe configured to hybridize to said amplification product; and wherein each of the at least five said reaction sites contains a different amplification primer pair with corresponding detectably labeled probe. In one embodiment, the support comprises at least ten said reaction sites, wherein each of the at least ten said reaction sites contains a different amplification primer pair with corresponding detectably labeled probe. In one embodiment, the support comprises at least fifteen said reaction sites, wherein each of the at least fifteen said reaction sites contains a different amplification primer pair with corresponding detectably labeled probe. In one embodiment, the support comprises at least seventeen said reaction sites, wherein each of the at least seventeen said reaction sites contains a different amplification primer pair with corresponding detectably labeled probe. In one embodiment of the support, said amplification product is between 56 to 105 nucleotides long. In one embodiment of the support, each of said reaction sites contains a pair of amplification primers and a probe configured to amplify at least a portion of a gene selected from Table 1 or a nucleic acid derivative of a gene listed in Table 1. In one embodiment of the support, each of said reaction sites contains a pair of amplification primers and a probe selected from an assay id listed in Table 1. In one embodiment of the support, an amplification primer pair of at least one reaction site includes a primer containing a nucleic acid sequence that is complementary or identical to portion of said corresponding target nucleic acid sequence. In one embodiment of the support, said corresponding target nucleic acid sequence contains a nucleic acid sequence that is identical or complementary to a nucleic acid sequence present in genomic DNA, RNA, miRNA, mRNA, cell-free DNA, circulating DNA or cDNA. In one embodiment of the support, said corresponding target nucleic acid sequence is present within or is derived from genomic DNA, RNA, miRNA, mRNA, cell-free DNA, circulating DNA or cDNA derived from a target microorganism. In one embodiment of the support, said target microorganism is selected from Table 1. In one embodiment of the support, two or more of said reaction sites contain a portion of the same nucleic acid sample. In one embodiment of the support, said nucleic acid sample is derived from a urine specimen. In one embodiment of the support, at least one of said reaction sites includes an amplification product. In one embodiment of the support, said amplification product of a reaction site includes a nucleic acid sequence that is complementary or identical to a portion of a gene listed in Table 1. In one embodiment of the support, said support includes between 10 and 10, 000 reaction sites containing different amplification products. In one embodiment of the support, said support includes reaction sites containing amplification products that are identical or complementary to all of the genes listed in Table 1. In one embodiment of the support, at least two of said reaction sites each contains a pair of amplification primers configured to amplify a different corresponding target nucleic acid sequence. In one embodiment of the support, said corresponding target nucleic acid sequence contains a portion of the nucleic acid sequence of a gene listed in Table 1 or its corresponding cDNA. In one embodiment of the support, said plurality of reaction sites include amplification products for all of the genes listed in Table 1 using a nucleic acid sample derived from a microorganism listed in Table 1. In one embodiment of the support, said plurality of reaction sites include amplification products for any combination of at least two of the genes listed in Table 1 using a nucleic acid sample derived from at least two microorganisms listed in Table 1. In one embodiment of the support, said detectably labeled probe of at least one of said reaction sites is configured to undergo cleavage by a polymerase having 5′ exonuclease activity. In one embodiment of the support, said detectably labeled probe of at least one said reaction sites contains a fluorescent label at its 5′ end and a quencher at its 3′ end. In one embodiment of the support, said detectably labeled probe further contains a minor groove binder (MGB) moiety. In one embodiment of the support, said support is selected from a multi-well plate, a microfluidic card, and a plate comprising a plurality of through-hole reaction sites. In one embodiment of the support, one or more of said individual reaction sites includes a dried deposit of a solution containing said pair of amplification primers and said detectably labeled probe. In one embodiment of the support, said individual reaction sites further include a polymerase and/or nucleotides. In one embodiment of the support, one or more of said individual reaction sites contains a lyophilized composition comprising said pair of amplification primers, said detectably labeled probe, a polymerase, and nucleotides. In one embodiment of the support, said amplification primer pair and said detectably labeled probe are from one of the assays listed in Table 1.

In yet another aspect, provided is a composition for determining the presence or absence of at least one target nucleic acid from one or more of the microorganisms listed in Table 1 in a biological sample, said composition comprising: (a) at least one amplification primer pair, wherein each of said primers of said pair comprises a target hybridization region that is configured to specifically hybridize to all or a portion of a region of said target nucleic acid and wherein under suitable conditions said primer pair generates an amplicon which from a gene in Table 1; and (b) at least one detection probe configured to specifically hybridize to all or a portion of a region of said amplicon produced by said primer pair. In one embodiment of the composition, said amplicon is between 56 to 105 nucleotides long. In one embodiment, the composition comprises at least one assay listed in Table 1. In one embodiment, the composition comprises a set of nucleotide probes for detecting a panel of biomarkers; said probes being complementary to DNA and/or RNA sequences of a group of genes; characterized in that said group of genes are selected from any combination of those listed in Table 1. In one embodiment of the composition, said set of probes consists of 1 to 17 different probes. In one embodiment of the composition, said group of genes consists of at five different genes selected from those listed in Table 1. In one embodiment of the composition, at least five (5) different target nucleic acids in a sample are amplified and detected, said target nucleic acids being from five (5) different microorganisms listed in Table 1. In one embodiment of the composition, said five target nucleic acids are amplified and detected using the assay listed for each of said five different microorganisms listed in Table 1. In one embodiment of the composition, said group of genes consists of at ten different genes selected from those listed in Table 1. In one embodiment of the composition, at least ten (10) different target nucleic acids in a sample are amplified and detected, said target nucleic acids being from 10 (10) different microorganisms listed in Table 1. In one embodiment of the composition, said ten target nucleic acids are amplified and detected using the assay listed for each of said ten different microorganisms listed in Table 1. In one embodiment of the composition, said group of genes consists of at fifteen different genes selected from those listed in Table 1. In one embodiment of the composition, at least fifteen (15) different target nucleic acids in a sample are amplified and detected, said target nucleic acids being from fifteen (15) different microorganisms listed in Table 1. In one embodiment of the composition, said fifteen target nucleic acids are amplified and detected using the assay listed for each of said fifteen different microorganisms listed in Table 1. In one embodiment of the composition, said group of genes consists of at seventeen different genes selected from those listed in Table 1. In one embodiment of the composition, at least seventeen (17) different target nucleic acids in a sample are amplified and detected, said target nucleic acids being from seventeen (17) different microorganisms listed in Table 1. In one embodiment of the composition, said seventeen target nucleic acids are amplified and detected using the assay listed for each of said seventeen different microorganisms listed in Table 1. In one embodiment, the composition further comprises a polymerase having 5′ nuclease activity. In some embodiments, the polymerase is thermostable. In some embodiments, the polymerase is Taq DNA polymerase. In one embodiment, the detection probes of the composition are TaqMan probes or 5′nuclease probes.

In another aspect, provided is a method of profiling a panel of biomarkers associated with a biological sample comprising: (a) obtaining said biological sample from a subject; (b) contacting at least some portion of said sample with at least five individual amplification reactions, each of said individual reactions comprising a set of target-specific primers and a polymerase; (c) amplifying at least one target sequence per individual reaction under amplification conditions able to produce an amplified product; (c) contacting each of said plurality of individual reactions with a detectably labeled probe specific for said amplified product produced by said target-specific primers; (d) determining the presence or absence of said amplified product in each of said plurality of individual amplification reactions to arrive at a biomarker profile for said biological sample, wherein said biomarkers are associated with the genes listed in Table 1. In one embodiment of the method, at least ten individual amplification reactions are contacted by the at least some portion of said sample. In one embodiment of the method, at least fifteen individual amplification reactions are contacted by the at least some portion of said sample. In one embodiment of the method, at least seventeen individual amplification reactions are contacted by the at least some portion of said sample. In one embodiment of the method, said biomarkers are associated with urogenital infection and/or microbiota. In one embodiment of the method, said panel comprises a set of 1 to 17 different biomarkers. In one embodiment of the method, said plurality of individual amplification reactions are on a solid support. In one embodiment of the method, each of said plurality of individual amplification reactions comprises a single assay selected from Table 1. In another aspect, provided is a method for amplifying a plurality of nucleic acid target sequences in a sample containing a control nucleic acid molecule, the method comprising: performing a plurality of amplification reactions in parallel, each of the plurality of amplification reactions including a portion of the sample and a pair of amplification primers configured to amplify a corresponding target sequence in the control nucleic acid molecule, wherein the control nucleic acid molecule contains a plurality of different target sequences; forming a plurality of different amplification products corresponding to at least two different target sequences in the control nucleic acid molecule; and determining the presence of at least two different amplification products in the amplification reactions. In one embodiment of the method, the control nucleic acid molecule contains at least five different target sequences from different microorganisms set forth in Table 1. In one embodiment of the method, the control nucleic acid molecule contains at least ten different target sequences from different microorganisms set forth in Table 1. In one embodiment of the method, the control nucleic acid molecule contains at least fifteen different target sequences from different microorganisms set forth in Table 1. In one embodiment of the method, the control nucleic acid molecule contains all the different target sequences from different microorganisms set forth in Table 1. In one embodiment of the method, the plurality of different target sequences is derived from genomic or transcriptomic sequences of different microorganisms set forth in Table 1. In one embodiment of the method, the plurality of different target sequences is derived from any number of microorganism genes selected from Table 1. In one embodiment of the method, the forming includes forming in parallel between 5 and 100 different amplification products. In one embodiment of the method, the forming includes forming in parallel between 10 and 50 different amplification products. In one embodiment of the method, at least one pair of amplification primers configured to amplify a corresponding target sequence includes primers containing a nucleic acid sequence that is complementary or identical to a portion of the corresponding target sequence. In one embodiment of the method, at least two of the plurality of amplification reactions each contains a pair of amplification primers configured to amplify a different corresponding target sequence. In one embodiment of the method, one or more amplification reactions of the plurality further contains a detectably labeled probe that includes a sequence that is identical or complementary to a portion of the corresponding target sequence. In one embodiment of the method, the detectably labeled probe of at least one amplification reaction is configured to undergo cleavage by a polymerase having 5′ exonuclease activity. In one embodiment of the method, the detectably labeled probe of at least one amplification reaction contains a fluorescent label at its 5′ end and a quencher at its 3′ end. In one embodiment of the method, the control nucleic acid molecule is a DNA plasmid. In one embodiment of the method, the DNA plasmid is linear. In one embodiment, the method further comprises preparing the sample containing the control nucleic acid molecule from cells prior to the performing of amplification reactions.

In yet another aspect, provided is a method for amplifying a plurality of nucleic acid target sequences in a sample containing a control nucleic acid molecule, the method comprising: distributing the sample into a plurality of reaction volumes, where the control nucleic acid molecule contains a plurality of different target sequences, and wherein the reaction volumes include at least two different pair of amplification primers configured to amplify a corresponding target sequence in the control nucleic acid molecule; performing amplification reactions in the reaction volumes and forming a plurality of different amplification products corresponding to at least two different target sequences in the control nucleic acid molecule; and determining the presence of at least two different amplification products in the amplification reactions.

In yet another aspect, provided is method for evaluating a plurality of amplification reactions, comprising: distributing portions of a nucleic acid sample to individual reaction chambers situated within or upon a support, wherein the nucleic acid sample contains a control nucleic acid molecule and wherein the control nucleic acid molecule contains a plurality of different target sequences; performing a plurality of parallel amplification reactions and forming a plurality of different target amplification products corresponding to at least two different target sequences in the control nucleic acid molecule in the individual reaction chambers, wherein each amplification reaction contains a pair of amplification primers configured to amplify a corresponding target sequence present within the control nucleic acid molecule, at least two of the amplification reactions containing amplification primers configured to amplify different corresponding target sequences present within the control nucleic acid molecule; and quantifying at least two different target amplification products formed in at least two of the individual reaction chambers. In one embodiment, the method is performed using a set of samples which are serial dilutions of the control nucleic acid molecule. In one embodiment, the method further comprises determining a limit of detection for at least one of the control nucleic acid molecule target sequences based on the quantified target amplification products from the serially diluted control nucleic acid molecule. In one embodiment, the method further comprises determining a dynamic range for at least one of the control nucleic acid molecule target sequences based on the quantified target amplification products from the serially diluted control nucleic acid molecule. In one embodiment of the method, the quantifying includes detecting hybridization of a detectably labeled probe to the amplification product, optionally in real time. In one embodiment of the method, the control nucleic acid molecule comprises at least five different target sequences from microorganisms set forth in Table 1. In one embodiment of the method, the control nucleic acid molecule contains at least ten different target sequences from microorganisms set forth in Table 1. In one embodiment of the method, the control nucleic acid molecule contains at least fifteen different target sequences from microorganisms set forth in Table 1. In one embodiment of the method, the control nucleic acid molecule contains about all the different target sequences from microorganisms set forth in Table 1. In one embodiment of the method, the plurality of target sequences are derived from genomic sequences of different microorganisms in Table 1. In one embodiment of the method, the forming includes forming between 5 and 100 different amplification products. In one embodiment of the method, the forming includes forming between 1 and 17 different amplification products. In one embodiment of the method, one or more amplification reactions of the plurality further contains a detectably labeled probe that includes a sequence that is identical or complementary to a portion of the corresponding target sequence. In one embodiment of the method, the detectably labeled probe of at least one amplification reaction is configured to undergo cleavage by a polymerase in having 5′ exonuclease activity. In one embodiment of the method, the detectably labeled probe of at least one amplification reaction contains a fluorescent label at its 5′ end and a quencher at its 3′ end. In one embodiment of the method, the individual reaction chambers further includes a polymerase and/or nucleotides, distributed to the individual reaction chamber either prior to or after the portion of the sample is distributed to the reaction chamber. In one embodiment of the method, the control nucleic acid molecule is a DNA plasmid. In one embodiment of the method, the DNA plasmid is linear.

In still another aspect, provided is a nucleic acid construct comprising a plurality of different amplification target sequences, wherein at least two of the amplification target sequences comprise at least a 56 nucleotide portion of a gene selected from Table 1 or its corresponding cDNA. In another aspect, provided is a nucleic acid construct comprising a plurality of different amplification target sequences, wherein at least two of the amplification target sequences are derived from at least two different microorganisms or microorganism genes selected from Table 1.

In another aspect, provided is an array for nucleic acid amplification, comprising: a support containing a plurality of reaction sites located within the support or upon the support; each of the plurality of reaction sites containing: (i) a control nucleic acid molecule containing a plurality of different target sequences, (ii) an amplification primer pair configured to amplify a corresponding target sequence, and (iii) a detectably labeled probe configured to hybridize to a nucleic acid sequence generated by extension of at least one of the amplification primers of the pair. In one embodiment of the array, at least two of the different target sequences comprise at least a 56 nucleotide portion of a gene selected from Table 1 or its corresponding cDNA. In one embodiment of the array, the control nucleic acid molecule comprises at least five different target sequences from microorganisms set forth in Table 1. In one embodiment of the array, the control nucleic acid molecule contains at least ten different target sequences from microorganisms set forth in Table 1. In one embodiment of the array, the control nucleic acid molecule contains at least fifteen different target sequences from microorganisms set forth in Table 1. In one embodiment of the array, the control nucleic acid molecule contains all of the different target sequences from microorganisms set forth in Table 1. In one embodiment of the array, the control nucleic acid molecule is a plasmid. In one embodiment of the array, the plasmid is linear. In one embodiment of the array, at least one of the reaction sites includes an amplification product. In one embodiment of the array, the support includes between 10 and 10,000 reaction sites containing different amplification products. In one embodiment of the array, at least two of the reaction sites each contains a pair of amplification primers configured to amplify a different corresponding target sequence. In one embodiment of the array, the detectably labeled probe of at least one reaction site is configured to undergo cleavage by a polymerase having 5′ exonuclease activity. In one embodiment of the array, the detectably labeled probe of at least one reaction site contains a fluorescent label at its 5′ end and a quencher at its 3′ end. In one embodiment of the array, the detectably labeled probe further contains a minor groove binder moiety. In one embodiment of the array, the support is selected from a multi-well plate, a microfluidic card, and a plate containing a plurality of through-hole reaction sites. In one embodiment of the array, the plurality of reaction sites further include a polymerase and/or nucleotides.

In yet another aspect, provided is a method for amplifying a plurality of nucleic acid target sequences, comprising: distributing both a control nucleic acid molecule and a test nucleic acid sample into a plurality of reaction volumes, where the control nucleic acid molecule includes a plurality of different target sequences and the test nucleic acid sample includes one or more test nucleic acid molecules; subjecting the reaction volumes to nucleic acid amplification conditions and amplifying at least two different target sequences of the control nucleic acid molecule in the reaction volumes using pairs of amplification primers, each pair of amplification primers being used to amplify a different target sequence in the control nucleic acid molecule; detecting the presence of at least two different amplified target sequences in the reaction volumes. In one embodiment of the method, the control nucleic acid molecule is circular. In one embodiment of the method, the control nucleic acid molecule is linear. In one embodiment, the method further comprises distributing the control nucleic acid molecule and a test nucleic acid molecule from the test nucleic acid sample to different reaction volumes. In one embodiment of the method, the test nucleic acid sample also includes two or more different target nucleic acid molecules, each containing a different target sequence. In one embodiment, the method further comprises amplifying at least two different target sequences of the test nucleic acid sample in the reaction volumes using pairs of amplification primers, each pair of amplification primers being used to amplify a different target sequence in the target nucleic acid sample.

In another aspect, provided is a method for detecting the presence of a uropathogen in a biological sample, the method comprising the use of at least one assay selected from Table 1. In one embodiment, the method comprises the use of at least 10, at least 15, or preferably, at least 17 assays selected from Table 1. In one embodiment, the method for detecting the presence of a uropathogen comprises the use of a method for synthesizing and/or amplifying a plurality of nucleic acid target sequences, as described herein. In some embodiments, the method for synthesizing and/or amplifying comprises a PCR. In some embodiments, the PCR is qPCR. In some embodiments, the synthesizing and/or amplifying is performed on a solid support, such as an TaqMan OpenArray. In some embodiments, the qPCR method for detecting the presence of a uropathogen in a biological sample provides results which are at least 2× more accurate and/or sensitive than results obtained using a traditional culture-based method. In some embodiments, the qPCR method for detecting the presence of a uropathogen in a biological sample provides results which are at least 3× more accurate and/or sensitive than results obtained using a traditional culture-based method. In some embodiments, accuracy and/or sensitivity of the method for detecting is verified using Sanger Sequencing methods.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a workflow 100 for amplifying a plurality of nucleic acid sequences in accordance with one embodiment.

FIG. 2 illustrates a reaction vessel 200 in accordance with one embodiment.

FIG. 3 illustrates a method 300 for amplifying a plurality of nucleic acid sequences in a nucleic acid sample in accordance with one embodiment.

FIG. 4 depicts serial dilution data for a panel of 17 UTM assays (see Table 1 for list of assays) and 2 control assays (RNaseP and Xeno) using a linearized control DNA plasmid (i.e., superplasmid) sample as the template at varying concentrations, as indicated.

FIG. 5 illustrates alternative ways to present copies/μl in the context of stock solution, PCR reaction per sub-array (5 μl) or per through-hole (33 nl).

FIG. 6 displays experimental results summarizing the R-square and slope of serial dilution assays for a panel of 17 UTM assays (see Table 1 for list of assays) and 2 control assays (RNaseP and Xeno) using a linearized control DNA plasmid (i.e., superplasmid) sample as the template at varying concentrations.

FIG. 7 depicts graphical results for the limit of detection and dynamic range for assays directed to a panel of nine different targets selected from the group of seventeen microorganisms listed in Table 1 using a linearized control DNA plasmid (i.e., superplasmid) sample as the template at varying concentrations. In each graph, the X-axis shows logio of the template concentration and the Y-axis shows the PCR Ct values.

FIG. 8 illustrates experimental results evaluating accuracy and specificity of 17 UTM assays (see Table 1 for list of assays) against an ATCC gDNA inclusivity panel.

FIG. 9 illustrates experimental results evaluating accuracy and specificity of 17 UTM assays (see Table 1 for list of assays) against an ATCC gDNA exclusivity panel.

FIG. 10 illustrates, from a collection of 115 urine samples, the number of samples identified as positive for a given uropathogen using a qPCR UTM assay as described herein or a culture-based method.

FIG. 11 illustrates experimental results evaluating accuracy and specificity of 17 urine samples using qPCR UTM assays (see Table 1 for list of assays). Urine specimens were analyzed using either qPCR UTM assays as described herein or a traditional culture method. Positive results for qPCR are shown as average Ct values (for assays run in triplicate) and are indicated by both dark and grey highlighted squares. Positive results for cultures are indicated by dark grey highlighted squares.

FIG. 12 shows orthogonal testing of urine samples by Sanger Sequencing to confirm positive OpenArray™ results for samples having negative or inconclusive culture growth.

FIGS. 13A and 13B illustrate concordance numbers between samples tested by qPCR UTM assays as described herein versus those analyzed by a traditional culture method.

### DETAILED DESCRIPTION

The present disclosure relates to methods, compositions, and kits for amplification and characterization of select sets of microorganisms in a biological sample. For example, embodiments disclosed herein provide methods, compositions, and kits for detecting and/or monitoring urogenital, bladder, and urinary tract microbiome constituents and dynamics.

The methods, compositions, and kits disclosed herein may be utilized for detection of healthy and pathogenic microorganisms of urinary flora, including a range of bacteria, fungi, protozoa, and viruses.

The methods, compositions, and kits provided herein can be of use in the detection of pathogens and microbiota associated with bacterial and fungal bladder and urinary tract infections (UTIs). Results from the methods and compositions may be of use in determining treatment regimen(s) suitable for the individual from which the examined sample was obtained. The methods and compositions provided herein may further be used to monitor the microbiota composition and/or dynamics during, and after, treatment of the individual.

A microorganism nucleic acid may be detected in a sample by subjecting the sample to multiple individual amplification reactions, each reaction performed with a pair of amplification primers designed to be specific for at least a portion of the target microbe nucleic acid, with a detectably labeled probe specific for the target sequence amplified by the primers. In some aspects, the multiple individual amplification reactions as disclosed herein can generate individual amplification products for each of the microbes for which amplification primers and detector probes are designed or configured. In some embodiments, the microbial profile of a sample is arrived at by determining the presence or absence (+ or −) of the targeted amplification products from the individual amplification reactions. In some embodiments, a plurality of individual amplification reactions, each comprising a different targeted amplification product, are analyzed simultaneously to arrive at a microbial profile of a given sample.

Detection assays may utilize oligonucleotide primers and a detectably labeled probe for amplification and detection of microbial species-specific gene targets. Some detection assays may utilize TaqMan® Gene Expression Assays for amplification and detection of microbial species-specific gene targets.

Additional amplification reactions and assays may be performed as reference and/or control reactions and assays. Without limitation, these reference and/or control reactions and assays can be used in relative quantification applications to assess the adequacy of the biological sample or the nucleic acid sample, to normalize microbial presence, and/or to detect the presence of amplification inhibitors in the biological or nucleic acid sample. Exemplary target nucleic acids for such reference and/or control assays include, without limitation, prokaryotic 16S rRNA, human RNase P gene DNA (RNaseP), added exogenous nucleic acid, and/or a xeno nucleic acid (Xeno; XNA).

The methods, compositions, and kits may in some cases be used in performing a plurality of single-plex nucleic acid amplification reactions under the same assay conditions and/or at substantially the same time.

Amplifying target sequences in a sample may involve contacting at least some portion of the sample with target-specific primers as disclosed herein and at least one polymerase under amplification conditions, thereby producing at least one amplified target sequence. This may involve contacting at least some portion of the sample with target-specific primer pairs and at least one polymerase under amplification conditions, thereby producing at least one amplified target sequence.

In some embodiments, the nucleic acid sequences in a nucleic acid sample may be aliquoted to form at least five different amplification reaction mixes, each including an aliquot from a sample source including nucleic acid sequences, using at least five different assays, each including a pair of amplification primers and a detectably-labeled probe. In some embodiments, the different assays are selected from the group of TaqMan® Gene Expression Assay IDs listed in Table 1.

TABLE 1
Microorganism Gene Target Regions and Assays

| | | | Accession # | Position of | Size of | Assay | |
|---|---|---|---|---|---|---|---|
| Type of | Microorganism- | | for target | target | target | Amplicon | |
| Microbe | Species | Gene targeted | region | region | region | Size | Assay ID |
| Bacterial- | Acinetobacter | unannotated region | NZ_ | 202100- | 701 | 75 | Ba04932084_ |
| Gram | baumannii (AB) | | GG704574.1 | 202800 | | | s1 |
| negative | | | | | | | |
| Bacterial- | Citrobacter | COG2140 Oxalate | NZ_ | 137400- | 801 | 103 | Ba04932088_ |
| Gram | freundii (CF) | decarboxylase/ | ANAV01000004.1 | 138200 | | | s1 |
| negative | | archaeal | | | | | |
| | | phosphoglucose | | | | | |
| | | isomerase, cupin | | | | | |
| | | superfamily | | | | | |
| Bacterial- | Citrobacter | iron complex | NZ_ | 277000- | 801 | 62 | Ba07286617_ |
| Gram | freundii (CF-1) | transport system | ANAV01000001.1 | 277800 | | | s1 |
| negative | | substrate-binding | | | | | |
| | | protein | | | | | |
| Bacterial- | Citrobacter | iron complex | NZ_ | 277000- | 801 | 110 | Ba07286616_ |
| Gram | freundii (CF-2) | transport system | ANAV01000001.1 | 277800 | | | s1 |
| negative | | substrate-binding | | | | | |
| | | protein | | | | | |
| Bacterial- | Enterobacter | pyridoxal | CP014748.1 | 1158600- | 801 | 98 | Ba04932080_ |
| Gram | aerogenes (EA)- | phosphate- | | 1159400 | | | s1 |
| negative | presently known | dependent histidine | | | | | |
| | as Klebsiella | decarboxylase | | | | | |
| | aerogenes (K4) | (hdc) gene | | | | | |
| Bacterial- | Enterobacter | hypothetical | CP008823.1 | 3274000- | 801 | 88 | Ba04932087_ |
| Gram | cloacae (EnC) | protein | | 3274800 | | | s1 |
| negative | | | | | | | |
| Bacterial- | Enterococcus | aminotransferase | HF558530.1 | 1769100- | 801 | 95 | Ba04646247_ |
| Gram positive | faecalis (EF_b) | class V | | 1769900 | | | s1 |
| Bacterial- | Enterococcus | PhnB-MerR | NZ_ | 17300- | 801 | 98 | Ba04932086_ |
| Gram | faecium (EF) | family | GL476131.1 | 18100 | | | s1 |
| negative | | transcriptional | | | | | |
| | | regulator | | | | | |
| Bacterial- | Escherichia coli | COG0789 DNA- | CP015843.2 | 4336000- | 701 | 63 | Ba04646242_ |
| Gram | (EsC) | binding | | 4336700 | | | s1 |
| negative | | transcriptional | | | | | |
| | | regulator MerR | | | | | |
| | | family (Zntr) | | | | | |
| Bacterial- | Klebsiella | parC (DNA | CP020358.1 | 2851700- | 901 | 93 | Ba04932079_ |
| Gram | oxytoca (KO) | topoisomerase IV | | 2852600 | | | s1 |
| negative | | subunit A) | | | | | |
| Bacterial- | Klebsiella | ACT like protein | CP007727.1 | 209000- | 801 | 56 | Ba04932083_ |
| Gram | pneumoniae | | | 209800 | | | s1 |
| negative | (KP) | | | | | | |
| Bacterial- | Morganella | COG1918-Fe2 + | CP004345.1 | 375800- | 801 | 91 | Ba04932078_ |
| Gram | morganii (MM) | transport system | | 376600 | | | s1 |
| negative | | protein FeoA | | | | | |
| Bacterial- | Proteus mirabilis | araC, ureR | CP017082.1 | 580200- | 801 | 100 | Ba04932076_ |
| Gram | (PM) | | | 581000 | | | s1 |
| negative | | | | | | | |
| Bacterial- | Proteus vulgaris | SUMF1 gene | JPIX01000006.1 | 102000- | 801 | 76 | Ba04932082_ |
| Gram | (PV) | | | 102800 | | | s1 |
| negative | | | | | | | |
| Bacterial- | Providencia | COG0641 | NZ_ | 493000- | 801 | 100 | Ba04932077_ |
| Gram | stuartii (PS) | Sulfatase | DS607663.1 | 493800 | | | s1 |
| negative | | maturation enzyme | | | | | |
| | | AslB, radical SAM | | | | | |
| | | superfamily, | | | | | |
| | | putative iron-sulfur | | | | | |
| | | modifier protein | | | | | |
| Bacterial- | Pseudomonas | N296_1760, helix | CP006831.1 | 1857600- | 801 | 70 | Ba04932081_ |
| Gram positive | aeruginosa (PA) | turn helix domain | | 1858400 | | | s1 |
| | | protein | | | | | |
| Bacterial- | Staphylococcus | cdaR | AP008934.1 | 200400- | 601 | 85 | Ba04932085_ |
| Gram positive | saprophyticus | | | 201000 | | | s1 |
| | (SS) | | | | | | |
| Bacterial- | Streptococcus | SIP | CP010319.1 | 41000- | 601 | 66 | Ba04646276_ |
| Gram positive | agalactiae (SA) | | | 41600 | | | s1 |
| Fungal/Yeast | Candida | IPT1 | AY884203.1 | 800- | 701 | 105 | Fn04646233_ |
| | albicans (CA) | | | 1500 | | | s1 |

In some aspects, each amplification reaction mix is applied to a reaction vessel, and thereafter amplification reactions are performed on the reaction vessel, followed by detection of amplification products corresponding to a target nucleic acid sequence within one or more locations on the reaction vessel during the amplification reactions. As disclosed herein, the reaction can be utilized in an amplification product detection system, operated to associate locations of the amplification reaction mix on the reaction vessel with one or more of the assay IDs utilized in the amplification reaction mix. In various embodiments, the reaction vessel can be a tube, a plate with wells, a card, an array, an open array, or a chip microarray. In some embodiments, the reaction vessel is a solid support (“support”). In some embodiments, the reaction vessel or support may further include a reaction site or a plurality of reaction sites. In some embodiments the reaction site may be, but is not limited to, a chamber, well, through-hole, spot, container or compartment located on or within any of the foregoing reaction vessels.

In some aspects, the methods involve forming a plurality of amplification reaction mixes each including an aliquot from a sample source including nucleic acid sequences, using a plurality of different assays selected from the group of assays in Table 1. In some embodiments, the methods involve forming at least five amplification reaction mixes each including an aliquot from a sample source including nucleic acid sequences, using at least five different assays selected from the group of assays (see list of Assay IDs) in Table 1. In other embodiments, the methods involve forming at least ten amplification reaction mixes each including an aliquot from a sample source including nucleic acid sequences, using at least ten different assays selected from the group of assays in Table 1; or forming at least fifteen amplification reaction mixes each including an aliquot from a sample source including nucleic acid sequences, using at least fifteen different assays selected from the group of assays in Table 1; or forming reaction mixes each including an aliquot from a sample source including nucleic acid sequences, using all of the assays in Table 1.

In some embodiments, the sample source is typically, though not necessarily, a urine specimen. In some embodiments, the urine specimen is collected by urine voiding, through the use of a catheter, or by suprapubic aspiration.

See Table 1 for assays that may be utilized in the reactions as disclosed herein. For example, in some embodiments, the assay ID Ba04932084_s1 may include a pair of primers targeting a portion of a nucleic acid sequence of an unannotated region of a gene of Acinetobacter baumannii. In other embodiments, the assay ID Ba04932088_s1 may include a pair of primers that targets a portion of a nucleic acid sequence of a COG2140 for Oxalate decarboxylase/archaeal phosphoglucose isomerase, cupin superfamily gene of Citrobacter freundii. In other embodiments, the assay ID Ba07286617_s1 and/or Ba07286616_s1 may include a pair of primers that targets a portion of a nucleic acid sequence of an iron complex transport system substrate-binding protein of Citrobacter freundii. In other embodiments, the assay ID Ba04932080_s1 may include a pair of primers that targets a portion of a nucleic acid sequence of a pyridoxal phosphate-dependent histidine decarboxylase(hdc) gene of Klebsiella aerogenes (previously known as Enterobacter aerogenes). In other embodiments, the assay ID Ba04932087_s1 may include a pair of primers that targets a portion of a nucleic acid sequence of a hypothetical protein of a gene of Enterobacter cloacae. In other embodiments, the assay ID Ba04646247_s1 may include a pair of primers that targets a portion of a nucleic acid sequence of an aminotransferase class V gene of Enterococcus faecalis. In other embodiments, the assay ID Ba04932086_s1 may include a pair of primers that targets a portion of a nucleic acid sequence of a PhnB-MerR family transcriptional regulator gene of Enterococcus faecium. In other embodiments, the assay ID Ba04646242_s1 may include a pair of primers that targets a portion of a nucleic acid sequence of a COG0789 DNA-binding transcriptional regulator MerR family (Zntr) gene of Escherichia coli. In other embodiments, the assay ID Ba04932079_s1 may include a pair of primers that targets a portion of a nucleic acid sequence of a parC (DNA topoisomerase IV subunit A) gene of Klebsiella oxytoca. In other embodiments, the assay ID Ba04932083_s1 may include a pair of primers that targets a portion of a nucleic acid sequence an act-like protein gene of Klebsiella pneumoniae. In other embodiments, the assay ID Ba04932078_s1 may include a pair of primers that targets a portion of a nucleic acid sequence of a COG1918 for Fe2+ transport system protein FeoA gene of Morganella morganii. In other embodiments, the assay ID Ba04932076_s1 may include a pair of primers that targets a portion of a nucleic acid sequence of an araC, ureR gene of Proteus mirabilis. In other embodiments, the assay ID Ba04932077_s1 may include a pair of primers that targets a portion of a nucleic acid sequence of a SUMF1 gene of Proteus vulgaris. In other embodiments, the assay ID Ba04932082_s1 may include a pair of primers that targets a portion of a nucleic acid sequence of a COG0641 for Sulfatase maturation enzyme As1B, radical SAM superfamily, putative iron-sulfur modifier protein gene of Providencia stuartii. In other embodiments, the assay ID Ba04932081_s1 may include a pair of primers that targets a portion of a nucleic acid sequence of a N296_1760, helix turn helix domain protein gene of Pseudomonas aeruginosa. In other embodiments, the assay ID Ba04932085_s1 may include a pair of primers that targets a portion of a nucleic acid sequence of a cdaR gene of Staphylococcus saprophyticus. In other embodiments, the assay ID Ba04646276_s1 may include to a pair of primers that targets a portion of a nucleic acid sequence of a SIP gene of Streptococcus agalactiae. In other embodiments, the assay ID Fn04646233_s1 may include a pair of primers that targets a portion of a nucleic acid sequence of an IPT1 gene of Candida albicans.

In some embodiments, target amplicons produced by amplification of a nucleic acid sample by use of a species-specific assay, such as those listed in Table 1, may have an amplicon length of between 20 to 200 nucleotides, between 30 to 150 nucleotides, between 40 to 120 nucleotides, or between 50 to 110 nucleotides, for example, between 56 to 105 nucleotides.

Amplifying nucleic acid sequences in a nucleic acid sample may in some embodiments involve forming amplification reaction mixes each including an aliquot from a sample source including nucleic acid sequences, wherein the sample source is a urine specimen, applying the amplification reaction mixes to a reaction vessel, where the reaction vessel is configured with at least five assays each targeting a different gene located within the corresponding target regions listed in Table 1. Each assay includes a pair of amplification primers, performing amplification reactions on the reaction vessel, and detection is performed for an amplification product corresponding to a target nucleic acid sequence within locations on the reaction vessel during the amplification reactions. In some embodiments, an amplification product detection system associates locations of the amplification reaction mix on or in a reaction vessel with one or more of the assay utilized on the reaction vessel. In various embodiments, the reaction vessel is a plate with wells, an array, an OpenArray with multiple through-holes, or a chip microarray.

In various embodiments, the reaction vessel may in some cases be configured with at least five assays each targeting a different gene listed in Table 1. In some embodiments, the reaction vessel may in some cases be configured with at least ten assays each targeting a different gene listed in Table 1, or with at least fifteen assays each targeting a different gene listed in Table 1, or with seventeen assays each targeting one of the genes listed in Table 1. In some embodiments, an assay in the at least five assays may amplify an amplicon that is 93 nucleotides long, in a gene corresponding to unannotated region in Acinetobacter baumannii. In some embodiments, an assay of the at least five assays may amplify an amplicon of that is 103 nucleotides long in a gene corresponding to COG2140 for Oxalate decarboxylase/archaeal phosphoglucose isomerase, cupin superfamily in Citrobacter freundii. In some embodiments, an assay of the at least five assays may amplify an amplicon of that is 62 and/or 110 nucleotides long in a gene corresponding to COG2140 for Oxalate decarboxylase/archaeal phosphoglucose isomerase, cupin superfamily in an iron complex transport system substrate-binding protein of Citrobacter freundii. In some embodiments, an assay of the at least five assays may amplify an amplicon that is 98 nucleotides long, in a gene corresponding to pyridoxal phosphate-dependent histidine decarboxylase (hdc) in Klebsiella aerogenes (previously known as Enterobacter aerogenes). In some embodiments, an assay of the at least five assays may amplify an amplicon that is 88 nucleotides long for a gene corresponding to a hypothetical protein in Enterobacter cloacae. In some embodiments, an assay of the at least five assays may amplify an amplicon that is 95 nucleotides long in a gene corresponding to aminotransferase class V in Enterococcus faecalis. In some embodiments, an assay of the at least five assays may amplify an amplicon that is 98 nucleotides long in a gene corresponding to PhnB-MerR family transcriptional regulator in Enterococcus faecium. In some embodiments, an assay of the at least five assays may amplify an amplicon that is 63 nucleotides long in a gene corresponding to COG0789 Dna-binding transcriptional regulator MerR family (Zntr) in Escherichia coli. In some embodiments, an assay of the at least five assays may amplify an amplicon that is 93 nucleotides long in a gene corresponding to parC (DNA topoisomerase IV subunit A) in Klebsiella oxytoca. In some embodiments, an assay of the at least five assays may amplify an amplicon that is 56 nucleotides long in a gene corresponding to act-like protein in Klebsiella pneumoniae. In some embodiments, an assay of the at least five assays may amplify an amplicon that is 91 nucleotides long in a gene corresponding to COG1918 for Fe2+ transport system protein FeoA in Morganella morganii. In some embodiments, an assay of the at least five assays may amplify an amplicon that is 100 nucleotides long in a gene corresponding to araC, ureR in Proteus mirabilis. In some embodiments, an assay of the at least five assays may amplify an amplicon that is 76 nucleotides long in a gene corresponding to SUMF1 in Proteus vulgaris. In some embodiments, an assay of the at least five assays may amplify an amplicon that is 100 nucleotides long in a gene corresponding to COG0641 for Sulfatase maturation enzyme As1B, radical SAM superfamily, putative iron-sulfur modifier protein in Providencia stuartii. In some embodiments, an assay of the at least five assays may amplify an amplicon that is 70 nucleotides long in a gene corresponding to N296_1760, helix turn helix domain protein in Pseudomonas aeruginosa. In some embodiments, an assay of the at least five assays may amplify an amplicon that is 85 nucleotides long in a gene corresponding to cdaR in Staphylococcus saprophyticus. In some embodiments, an assay of the at least five assays may amplify an amplicon that is 66 nucleotides long in a gene corresponding to SIP in Streptococcus agalactiae. In some embodiments, an assay of the at least five assays may amplify an amplicon that is 105 nucleotides long in a gene corresponding to IPT1 in Candida albicans. In some embodiments, at least one assay of the at least five; at least ten; or at least fifteen assays is selected from any of the three assays IDs highlighted in bold text in Table 1. In some embodiments, at least one assay of the at least five; at least ten; or at least fifteen assays is specific to any of the three microorganism-species highlighted in bold text in Table 1. In some embodiments, at least one assay of the at least five; at least ten; or at least fifteen assays is selected from any of the three assay IDs highlighted in bold text in Table 1. In some embodiments, at least one assay of the at least five; at least ten; or at least fifteen assays is selected from any of the three assay IDs listed in Table 1 for Enterobacter cloacae (EnC), Proteus vulgaris (PV), and/or Providencia stuartii (PS).

In accordance with these methods, a composition for determining the presence or absence of at least one target nucleic acid in a biological sample includes at least five different amplification primer pairs, wherein each of the primers of the pairs include a target hybridization region that is configured to specifically hybridize to all or a portion of a region of a nucleic acid sequence of a target microorganism in Table 1 and wherein under suitable conditions the primer pair generates an amplicon, and wherein at least five detection probes configured to specifically hybridize to all or a portion of a region of the amplicon produced by the primer pair. In some embodiments, the composition includes a control nucleic acid molecule including different nucleic acid target sequences; the target nucleic acid sequences specific to at least five of the genes in Table 1. In some embodiments, the control nucleic acid molecule is a DNA plasmid comprising a plurality of target nucleic acid sequences specific to different genes listed in Table 1 (e.g., at least five, at least ten, or at least fifteen different genes, or all of the genes listed in Table 1). In some embodiments, the composition is a panel or collection of assays, for example a panel or collection of TaqMan® Assays. In some embodiments, the composition is a panel or collection of assays, for example a panel or collection of TaqMan® Assays including at least five; at least ten; at least fifteen; or all of the TaqMan® Gene Expression Assays (having the specific assay IDs) listed in Table 1. In some embodiments, the panel or collection of assays comprise a plurality of TaqMan® Gene Expression Assays. In some embodiments, the panel or collection of assays comprise a plurality of TaqMan® Gene Expression Assays obtained from or supplied by Thermo Fisher Scientific.

The at least one target nucleic acid may be a biomarker for a microbe associated with a urinary tract infection, and/or the composition may be a solid support. The at least five amplification primer pairs are separated by location on the solid support. In other embodiments, the composition includes at least ten, at least fifteen, or seventeen different amplification primer pairs, wherein each of the primers of the pair includes a target hybridization region that is configured to specifically hybridize to all or a portion of a region of a nucleic acid sequence of targeted genes in Table 1, and wherein under suitable conditions the primer pair generates an amplicon. In some embodiments, the associated amplicon generated from the pair of primers configured to specifically hybridize to all or a portion of a region of a nucleic acid sequence of the targeted genes in Table 1 have the indicated size relative to each corresponding assay listed in Table 1.

In some aspects, the methods provided herein utilize an assay comprising an oligonucleotide primer or set of primers and/or a probe which is designed to hybridize to a target nucleic acid sequence (or a complementary sequence) within a target nucleic acid region (“target region”). In some embodiments, the target region is within a larger sequence associated with a particular accession number. In some embodiments, the target region may be between 500 to 1000 nucleotides long. In some embodiments, the target region is selected from any of those target regions listed in Table 1. For example, in some embodiments, a target nucleic acid sequence for a selected microorganism species may be within a target region within a sequence associated with a particular accession number, said region having an identifiable position within said sequence associated with the accession number. In some embodiments, a target nucleic acid sequence for Acinetobacter baumannii may be within a 701 nucleic acid sequence in accession number NZ GG704574.1 positioned in a region corresponding to nucleotides 202100-202800 of the genome. In some embodiments, a target nucleic acid sequence for Citrobacter freundii may be within a 801 nucleic acid sequence in accession number NZ_ANAV01000004 .1 positioned in a region corresponding to nucleotides 137400-138200 of the genome. In some embodiments, a target nucleic acid sequence for Citrobacter freundii may be within a 801 nucleic acid sequence in accession number NZ_ANAV01000001.1 positioned in a region corresponding to nucleotides 277000-277800 of the genome. In some embodiments, a target nucleic acid sequence for Klebsiella aerogenes (previously known as Enterobacter aerogenes) may be within a 801 nucleic acid sequence in accession number CP014748 .1 positioned in a region corresponding to nucleotides 1158600-1159400 of the genome. In some embodiments, a target nucleic acid sequence for Enterobacter cloacae may be within a 801 nucleic acid sequence in accession number CP008823 .1 positioned in a region corresponding to nucleotides 3274000-3274800 of the genome. In some embodiments, a target nucleic acid sequence for Enterococcus faecalis may be within a 801 nucleic acid sequence in accession number HF558530 .1 positioned in a region corresponding to nucleotides 1769100-1769900 of the genome. In some embodiments, a target nucleic acid sequence for Enterococcus faecium may be within a 801 nucleic acid sequence in accession number NZ_GL476131 .1 positioned in a region corresponding to nucleotides 17300-18100 of the genome. In some embodiments, a target nucleic acid sequence for Escherichia coli may be within a 701 nucleic acid sequence in accession number CP015843 .2 positioned in a region corresponding to nucleotides 4336000-4336700 of the genome. In some embodiments, a target nucleic acid sequence for Klebsiella oxytoca may be within a 801 nucleic acid sequence in accession number CP020358 .1 positioned in a region corresponding to nucleotides 2851700-2852600 of the genome. In some embodiments, a target nucleic acid sequence for Klebsiella pneumoniae may be within a 801 nucleic acid sequence in accession number CP007727.1 positioned in a region corresponding to nucleotides 209000-2090800 of the genome. In some embodiments, a target nucleic acid sequence for Morganella morganii may be within a 801 nucleic acid sequence in accession number CP004345 .1 positioned in a region corresponding to nucleotides 375800-376600 of the genome. A target nucleic acid sequence for Proteus mirabilis may be within a 801 nucleic acid sequence in accession number CP017082 .1 positioned in a region corresponding to nucleotides 580200-581000 of the genome. In some embodiments, a target nucleic acid sequence for Proteus vulgaris may be within a 801 nucleic acid sequence in accession number JPIX01000006 0.1 positioned in a region corresponding to nucleotides 10200-102800 of the genome. In some embodiments, a target nucleic acid sequence for Providencia stuartii may be within a 801 nucleic acid sequence in accession number NZ_DS607663 .1 positioned in a region corresponding to nucleotides 493000-493800 of the genome. In some embodiments, a target nucleic acid sequence for Pseudomonas aeruginosa may be within a 801 nucleic acid sequence in accession number CP006831.1 positioned in a region corresponding to nucleotides 1857600-1858400 of the genome. In some embodiments, a target nucleic acid sequence for Staphylococcus saprophyticus may be within a 601 nucleic acid sequence in accession number AP008934 .1 positioned in a region corresponding to nucleotides 200400-201000 of the genome. In some embodiments, a target nucleic acid sequence for Streptococcus agalactiae may be within a 601 nucleic acid sequence in accession number CP010319 .1 positioned in a region corresponding to nucleotides 41000-41600 of the genome. In some embodiments, a target nucleic acid sequence for Candida albicans may be within a 701 nucleic acid sequence in accession number AY884203 .1 positioned in a region corresponding to nucleotides 800-1500 of the genome.

In some embodiments, a nucleic acid construct for evaluating amplification reactions may thus be utilized, which includes a control nucleic acid molecule including different nucleic acid target sequences directed to at least five of the genes targeted in Table 1 inserted into a DNA plasmid. The plurality of target nucleic acid sequences may in some cases be directed to at least ten of the genes in Table 1 inserted into a DNA plasmid, or to at least fifteen of the genes in Table 1 inserted into a DNA plasmid, or to each of the genes in Table 1 inserted into a DNA plasmid. In some embodiments, the DNA plasmid comprising a plurality of nucleic acid target sequences can be used as a positive control nucleic for amplification.

In some embodiments, the DNA plasmid comprising a plurality of target nucleic acid sequences (“superplasmid”), can comprise at least five, at least ten, at least fifteen, or seventeen different nucleic acid target sequences which are identical to or complementary to amplicons generated by use of an assay selected from those listed in Table 1. In some embodiments, the DNA plasmid may comprise a target nucleic acid sequence for Acinetobacter baumannii that is within a 701 nucleic acid sequence in accession number NZ_GG704574 .1 positioned in a region corresponding to nucleotides 202100-202800 of the genome. In some embodiments, the DNA plasmid may comprise a target nucleic acid sequence for Citrobacter freundii that is within a 801 nucleic acid sequence in accession number NZ_ANAV01000004 .1 positioned in a region corresponding to nucleotides 137400-138200 of the genome. In some embodiments, the DNA plasmid may comprise a target nucleic acid sequence for Citrobacter freundii that is within a 801 nucleic acid sequence in accession number NZ_ANAV01000001.1 positioned in a region corresponding to nucleotides 277000-277800 of the genome. In some embodiments, the DNA plasmid may comprise a target nucleic acid sequence for Klebsiella aerogenes (previously known as Enterobacter aerogenes) that is within a 801 nucleic acid sequence in accession number CP014748 .1 positioned in a region corresponding to nucleotides 1158600-1159400 of the genome. In some embodiments, the DNA plasmid may comprise a target nucleic acid sequence for Enterobacter cloacae that is within a 801 nucleic acid sequence in accession number CP008823 .1 positioned in a region corresponding to nucleotides 3274000-3274800 of the genome. In some embodiments, the DNA plasmid may comprise a target nucleic acid sequence for Enterococcus faecalis that is within a 801 nucleic acid sequence in accession number HF558530 .1 positioned in a region corresponding to nucleotides 1769100-1769900 of the genome. In some embodiments, the DNA plasmid may comprise a target nucleic acid sequence for Enterococcus faecium that is within a 801 nucleic acid sequence in accession number NZ_GL476131 .1 positioned in a region corresponding to nucleotides 17300-18100 of the genome. In some embodiments, the DNA plasmid may comprise a target nucleic acid sequence for Escherichia coli that is within a 701 nucleic acid sequence in accession number CP015843 .2 positioned in a region corresponding to nucleotides 4336000-4336700 of the genome. In some embodiments, the DNA plasmid may comprise a target nucleic acid sequence for Klebsiella oxytoca that is within a 801 nucleic acid sequence in accession number CP020358 .1 positioned in a region corresponding to nucleotides 2851700-2852600 of the genome. In some embodiments, the DNA plasmid may comprise a target nucleic acid sequence for Klebsiella pneumoniae that is within a 801 nucleic acid sequence in accession number CP007727 .1 positioned in a region corresponding to nucleotides 209000-2090800 of the genome. In some embodiments, the DNA plasmid may comprise a target nucleic acid sequence for Morganella morganii that is within a 801 nucleic acid sequence in accession number CP004345 .1 positioned in a region corresponding to nucleotides 375800-376600 of the genome. In some embodiments, the DNA plasmid may comprise a target nucleic acid sequence for Proteus mirabilis that is within a 801 nucleic acid sequence in accession number CP017082 .1 positioned in a region corresponding to nucleotides 580200-581000 of the genome. In some embodiments, the DNA plasmid may comprise a target nucleic acid sequence for Proteus vulgaris that is within a 801 nucleic acid sequence in accession number JPIX01000006 .1 positioned in a region corresponding to nucleotides 10200-102800 of the genome. In some embodiments, the DNA plasmid may comprise a target nucleic acid sequence for Providencia stuartii that is within a 801 nucleic acid sequence in accession number NZ DS607663 .1 positioned in a region corresponding to nucleotides 493000-493800 of the genome. In some embodiments, the DNA plasmid may comprise a target nucleic acid sequence for Pseudomonas aeruginosa that is within a 801 nucleic acid sequence in accession number CP006831 .1 positioned in a region corresponding to nucleotides 1857600-1858400 of the genome. In some embodiments, the DNA plasmid may comprise a target nucleic acid sequence for Staphylococcus saprophyticus that is within a 601 nucleic acid sequence in accession number AP008934 .1 positioned in a region corresponding to nucleotides 200400-201000 of the genome. In some embodiments, the DNA plasmid may comprise a target nucleic acid sequence for Streptococcus agalactiae that is within a 601 nucleic acid sequence in accession number CP010319 .1 positioned in a region corresponding to nucleotides 41000-41600 of the genome. In some embodiments, the DNA plasmid may comprise a target nucleic acid sequence for Candida albicans that is within a 701 nucleic acid sequence in accession number AY884203 .1 positioned in a region corresponding to nucleotides 800-1500 of the genome.

A method for amplifying nucleic acid sequences in a nucleic acid sample may thus involve performing amplification reactions, the amplification reactions each including a portion of a nucleic acid sample and a pair of amplification primers each configured to produce an amplification product corresponding to a different target nucleic acid sequence from a group of target nucleic acid sequences including the microorganisms and corresponding amplicon sizes, regions, and accession numbers set forth in Table 1, forming different amplification products from the amplification reactions, and determining the presence or absence of at least one of the different amplification products. The disclosed methods may utilize at least five, at least ten, at least fifteen, or all of the amplification reactions targeting nucleic acid sequences for the organisms and corresponding amplicon sizes, regions, and accession numbers set forth in Table 1.

In some embodiments, a method for amplifying nucleic acid sequences in a nucleic acid sample involves (a) performing amplification reactions including a portion of a nucleic acid sample and a pair of amplification primers configured to produce an amplification product corresponding to the target nucleic acid sequence, wherein each target nucleic acid sequence is the amplification product of a different gene set forth in Table 1, (b) forming different amplification products, and (c) and determining the presence or absence of at least one of the different amplification products, wherein at least five, at least ten, at least fifteen, or all of the amplification reactions include a pair of amplification primers selected from an assay ID listed in Table 1.

In some embodiments, the forming may include forming in parallel between 5 and 100 different amplification products, or forming in parallel between 10 and 50 different amplification products.

In some embodiments, the amplification product or amplicon may be between about 50 to 110 nucleotides long. For example, between 56 to 105 nucleotides long. In some embodiments, a pair of amplification primers may produce an amplification product that includes a nucleic acid sequence that is complementary or identical to a portion or all of the corresponding target nucleic acid sequence. In some embodiments, the corresponding target nucleic acid sequence may include a nucleic acid sequence that is identical or complementary to a nucleic acid sequence present in genomic DNA, RNA, miRNA, mRNA, cell-free DNA, circulating DNA and/or cDNA. The corresponding target nucleic acid sequence may be present within or be derived from genomic DNA, RNA, miRNA, mRNA, cell-free DNA, circulating DNA or cDNA of a target microorganism, where the target microorganism is a microorganism listed in Table 1. In some embodiments, the method may produce, in parallel, between 10 and 10, 000 different amplification products. At least two of the amplification reactions may each contain a pair of amplification primers configured to amplify a different corresponding target nucleic acid sequence. The corresponding target nucleic acid sequence may include a portion of a nucleic acid sequence of a gene listed in Table 1 or its corresponding cDNA. The gene will typically be present within a microorganism listed in Table 1. In some embodiments, each of the amplification reactions may include a set of amplification primers configured to produce an amplification product that is between about 50 to 110 nucleotides long, and/or may form one or more of the amplification products including a nucleic acid sequence that is complementary or identical to a portion of a gene listed in Table 1. In some embodiments, a separate amplification product is formed for at least five, at least ten, at least fifteen, or for all of the genes listed in Table 1 using a nucleic acid sample derived from at least five, at least ten, at least fifteen, or for each microorganism listed in Table 1. One or more of the amplification reactions may further include a detectably labeled probe that includes a sequence that is identical or complementary to a portion of the corresponding target nucleic acid sequence and/or amplification product (e.g., amplicon). In some embodiments, the detectably labeled probe may be configured to undergo cleavage by a polymerase having 5′ exonuclease activity. In some embodiments, the detectably labeled probe may include a fluorescent label at its 5′ end and a quencher at its 3′ end. In yet other embodiments, the detectably labeled probe may further contain a minor groove binder (MGB) moiety. In some embodiments, at least one of the amplification reactions may occur at an individual reaction site present within or upon a reaction vessel, the reaction vessel including one or more individual reaction sites.

In some embodiments, the reaction vessel may be selected from a multi-well plate, a microfluidic card, and a plate including through-hole reaction sites. The individual reaction site may include one or more of the amplification primers, and the amplifying further may involve distributing a portion of the nucleic acid sample to the individual reaction site. The individual reaction site may include a dried deposit of a solution including a pair of amplification primers and a nucleic acid probe, wherein the primers and probe are both configured to amplify a nucleic acid sequence derived from a gene listed in Table 1. The individual reaction site may further include a polymerase and nucleotides, either prior to or after a portion of the nucleic acid sample is distributed to the reaction site. The nucleic acid sample may be prepared from a urine specimen.

Methods for detecting the presence of a microorganism nucleic acid in a sample may thus involve (a) distributing portions of a nucleic acid sample to individual reaction sites or chambers situated within a reaction vessel or support, (b) performing parallel amplification reactions and forming at least five amplification products, each in individual reaction sites or chambers, wherein each amplification reaction may include a pair of amplification primers configured to produce an amplification product corresponding to a target nucleic acid sequence present within, or derived from, the genome of a microorganism, wherein the corresponding target nucleic acid sequence may include a portion of the nucleic acid sequence of a gene listed in Table 1 or its corresponding cDNA, and (c) determining whether the amplification product has been formed in one or more of the individual reaction sites or chambers, wherein at least five of the amplification reactions include a pair of amplification primers selected from an assay id listed in Table 1. In other embodiments, at least ten, or fifteen, or all of the amplification reactions include a pair of amplification primers selected from an assay id listed in Table 1.

Hybridization of a detectably labeled probe to the amplification product may be detected, optionally in real-time. At least one pair of the amplification primers may be configured to produce an amplification product corresponding to the target nucleic acid sequence includes primers including a nucleic acid sequence that is complementary or identical to a portion of the corresponding target nucleic acid sequence. The corresponding target nucleic acid sequence for at least one pair of the amplification primers may contain a nucleic acid sequence that is identical or complementary to a nucleic acid sequence present in genomic DNA, RNA, miRNA, mRNA, cell-free DNA, circulating DNA and/or cDNA. The corresponding target nucleic acid sequence may be present within or is derived from genomic DNA, RNA, miRNA, mRNA, cell-free DNA, circulating DNA and/or cDNA of a target microorganism. In various embodiments, the microorganism is a microorganism species listed in Table 1.

The individual reaction sites or chambers used to carry out these methods may include a polymerase and nucleotides, added or distributed to the reaction site either prior to or after the portion of the nucleic acid sample is distributed to the reaction site.

In some embodiments, a reaction vessel or support for nucleic acid amplification may involve reaction sites located within the vessel or support or on the support' s surface. In some embodiments, at least five, at least ten, at least fifteen, or all of the reaction sites include (1) a different amplification primer pair to produce an amplification product corresponding to a target nucleic acid sequence, wherein the amplification product corresponds to a microorganism in Table 1. In some other embodiments, the at least five, at least ten, at least fifteen, or all of the reaction sites further include (2) a detectably labeled probe configured to hybridize to the amplification product. Thus in some embodiments, the at least five, at least ten, at least fifteen, or each of the reaction sites may include a different amplification primer pair with a corresponding detectably labeled probe specific to the amplification product or amplicon generated by the amplification primer pair.

In some embodiments, each of the reaction sites may contain a pair of amplification primers and a probe configured to amplify at least a portion of a gene selected from Table 1 or a nucleic acid derivative of a gene listed in Table 1. In some embodiments, each of the reaction sites may include a pair of amplification primers and a probe selected from an assay id listed in Table 1.

A composition for determining the presence or absence of at least one target nucleic acid from one or more of the microorganisms listed in Table 1 in a biological sample may include (a) at least one amplification primer pair, wherein each of the primers of the pair includes a target hybridization region that is configured to specifically hybridize to all or a portion of a region of the target nucleic acid and wherein under suitable conditions the primer pair generates an amplicon from a gene in Table 1, and (b) at least one detection probe configured to specifically hybridize to all or a portion of a region of the amplicon produced by the primer pair. As in other embodiments, the amplicon may be between about 50 to 110 nucleotides long, for example between 56 to 105 nucleotides long, and the composition may include at least one assay listed in Table 1. The composition may include a set of nucleotide probes for detecting a panel of biomarkers, the probes being complementary to DNA and/or RNA sequences of a group of genes, and characterized in that the group of genes are selected from any combination of those listed in Table 1. The set of probes may consist of 1 to 17 different probes. The group of genes may include at least five (5) different genes selected from those listed in Table 1. In some embodiments, at least five (5) different target nucleic acids in a sample are amplified and detected, the target nucleic acids being from five (5) different microorganisms listed in Table 1 (other embodiments may target 10, 15, or 17 of the organisms in Table 1). In some embodiments, the target nucleic acids are amplified and detected using the assay listed for each of five different microorganisms listed in Table 1. In some embodiments, the target nucleic acids are amplified to produce the corresponding amplicons having the amplicon size listed in Table 1.

In some embodiments, a biological sample is obtained from a subject and at least some portion of the biological sample is contacted with an individual amplification reaction, wherein at least one target sequence per individual reaction is amplified to produce an amplified product. In some embodiments, the biological sample is contacted with at least five, at least ten, or at least fifteen individual amplification reactions, wherein at least one target sequence per individual reaction is amplified to produce at least five, at least ten, at least fifteen amplified products. In some other embodiments, each of the individual reactions is contacted with a detectably labeled probe specific for the amplified product produced by the target-specific primers, and the presence or absence of the amplified product in each of the individual amplification reactions is determined. In some embodiments, the presence or absence of the amplified product in each of the individual amplification reactions is used to arrive at a biomarker profile for the biological sample, wherein the biomarkers are associated with any of the genes listed in Table 1. In some embodiments, the biomarkers are associated with at least five, at least ten, at least fifteen or all of the genes listed in Table 1.

The biomarkers are associated with bladder, urinary tract and/or urogenital infection and/or microbiota. The panel can include a set of 1 to 17 different biomarkers. The individual amplification reactions may be on a solid support, with each of the individual amplification reactions utilizing a single different assay selected from Table 1.

The amplification reactions may be performed in parallel, each including a portion of the sample and a pair of amplification primers configured to amplify a corresponding target sequence in a control nucleic acid molecule, wherein the control nucleic acid molecule may include different target sequences. In some embodiments, different amplification products corresponding to different target sequences in the control nucleic acid molecule are formed, and the presence of at least two different amplification products in the amplification reactions is determined. In various embodiments, the control nucleic acid molecule may include at least five, at least ten, at least fifteen, or all of the different target sequences from different microorganisms set forth in Table 1.

In some embodiments, the different target sequences are derived from genomic or transcriptomic sequences of different microorganisms as set forth in Table 1, and more specifically, the target genes selected listed in Table 1. At least one pair of amplification primers may be configured to amplify a corresponding target sequence includes primers including a nucleic acid sequence that is complementary or identical to a portion of the corresponding target sequence. At least two of the amplification reactions may each may include a pair of amplification primers configured to amplify a different corresponding target sequence.

One or more of the amplification reactions may include a detectably labeled probe that includes a sequence that is identical or complementary to a portion of the corresponding target sequence. The detectably labeled probe of at least one amplification reaction may be configured to undergo cleavage by a polymerase having 5′ exonuclease activity. In some embodiments, the detectably labeled probe of at least one amplification reaction may include a fluorescent label at its 5′ end and a quencher at its 3′ end. The control nucleic acid molecule may be a DNA plasmid (e.g., superplasmid), and in some cases the plasmid or superplasmid is linear. The sample including the control nucleic acid molecule may be prepared from cells prior to the performing of amplification reactions.

In some embodiments, the method for amplifying nucleic acid target sequences in a sample including a control nucleic acid molecule may involve distributing a sample into reaction volumes, where a control nucleic acid molecule may include different target sequences, and wherein the reaction volumes include at least two different pairs of amplification primers configured to amplify a corresponding target sequence in the control nucleic acid molecule. In some embodiments, amplification reactions are performed in the reaction volumes and different amplification products corresponding to at least two different target sequences in the control nucleic acid molecule are formed. The presence of at least two different amplification products in the amplification reactions can then be determined.

Portions of a nucleic acid sample may be distributed to individual reaction chambers situated within or upon a support, wherein the nucleic acid sample may include a control nucleic acid molecule and wherein the control nucleic acid molecule may include different target sequences. In some embodiments, amplification reactions are performed in parallel and different target amplification products are formed corresponding to at least two different target sequences in the control nucleic acid molecule in individual reaction chambers, wherein each amplification reaction contains a pair of amplification primers configured to amplify a corresponding target sequence present within the control nucleic acid molecule, and at least two of the amplification reactions including amplification primers are configured to amplify different corresponding target sequences present within the control nucleic acid molecule. At least two different target amplification products formed in at least two of the individual reaction chambers can be quantified. The method may be performed using a set of samples which are serial dilutions of the control nucleic acid molecule.

A detection limit may be determined for at least one of the control nucleic acid molecule target sequences based on the quantified target amplification products from the serially diluted control nucleic acid molecule.

A dynamic range for at least one of the control nucleic acid molecule target sequences may be determined based on the quantified target amplification products from the serially diluted control nucleic acid molecule.

In some embodiments, quantifying may involve detecting hybridization of a detectably labeled probe to the amplification product, optionally in real time. The control nucleic acid molecule may include at least two, at least five, at least ten, at least fifteen, or all of the different target sequences from microorganisms set forth in Table 1. The target sequences may be derived from the genomic sequences of different microorganisms listed in Table 1. Between 1 and 17 different amplification products may be formed. One or more amplification reactions of a plurality of amplification reaction may further include a detectably labeled probe that comprises a sequence that is identical or complementary to a portion of the corresponding target sequence. The detectably labeled probe of at least one amplification reaction may be configured to undergo cleavage by a polymerase have 5′ exonuclease activity. The detectably labeled probe of at least one amplification reaction may include a fluorescent label at its 5′ end and a quencher at its 3′ end. The individual reaction chambers may further include a polymerase and nucleotides, added to the reaction chambers either prior to or after a portion of the sample is distributed to the reaction chamber. The control nucleic acid molecule may be a DNA plasmid, for example a linear plasmid, such a superplasmid as described herein.

The nucleic acid construct may include different amplification target sequences, wherein at least two of the amplification target sequences include at least a 56 nucleotide portion of a gene selected from Table 1 or its corresponding cDNA.

The nucleic acid construct may include different amplification target sequences, wherein at least two of the amplification target sequences are derived from at least two different microorganisms or microorganism genes selected from Table 1.

A support for nucleic acid amplification can be an array. In some embodiments, the array may include reaction sites located within the array or on the array. In some embodiments, each of the reaction sites can include (i) an amplification primer pair configured to amplify a corresponding target sequence, and (ii) a detectably labeled probe configured to hybridize to a nucleic acid sequence generated by extension of at least one of the amplification primers of the pair. In some other embodiments, at least one of the reaction sites can further include (iii) a control nucleic acid molecule including different target sequences. At least two of the different target sequences may include at least a 56 nucleotide portion of a gene selected from Table 1 or its corresponding cDNA. The control nucleic acid molecule may include at least two, at least five, at least ten, at least fifteen, or all of the different target sequences from microorganisms set forth in Table 1. The control nucleic acid molecule may be a plasmid, for example a plasmid that is linear, such a superplasmid as described herein. In some embodiments, at least one of the reaction sites includes an amplification product.

The support may include between 10 and 10,000 reaction sites including different amplification products. In some embodiments, at least two of the reaction sites each include a pair of amplification primers configured to amplify a different corresponding target sequence. The detectably labeled probe of at least one reaction site may be configured to undergo cleavage by a polymerase using a 5′ nuclease assay. The detectably labeled probe of at least one reaction site may contain a fluorescent label at its 5′ end and a quencher at its 3′ end. The detectably labeled probe may further include a minor groove binder moiety. The support may be selected from a multi-well plate, a microfluidic card, and a plate including through-hole reaction sites. The reaction sites may further include a polymerase and/or nucleotides.

A method for amplifying nucleic acid target sequences may involve distributing a control nucleic acid molecule and/or a test nucleic acid sample into reaction volumes, where the control nucleic acid molecule includes different target sequences and the test nucleic acid sample includes one or more test nucleic acid molecules, subjecting the reaction volumes to nucleic acid amplification conditions and amplifying at least two different target sequences of the control nucleic acid molecule in the reaction volumes using pairs of amplification primers, each pair of amplification primers being used to amplify a different target sequence in the control nucleic acid molecule, and detecting the presence of at least two different amplified target sequences in the reaction volumes. The control nucleic acid molecule may be circular, or linear. The control nucleic acid molecule and a test nucleic acid sample may be distributed to different reaction sites. The test nucleic acid sample may also include two or more different target nucleic acid molecules, each including a different target sequence. At least two different target sequences of the test nucleic acid sample in the reaction volumes may be amplified using pairs of amplification primers, each pair of amplification primers being used to amplify a different target sequence in the target nucleic acid sample.

In some embodiments, a reaction mixture is formed by contacting at least some portion of a nucleic acid sample with a target-specific primer pair and probe set (or assay) of Table 1 and at least one polymerase. In some embodiments, the reaction mixture is incubated under amplification conditions thereby producing at least one amplified target sequence. In some additional embodiments, the at least one amplified target sequence is detected and the presence the amplified target sequence in the nucleic acid sample is determined. Each target-specific primer and probe set may include a forward primer and a reverse primer designed to specifically amplify a target sequence and a detectably labeled probe specific to the nucleic acid amplified by the forward and reverse primers.

The methods, compositions and kits disclosed herein may be utilized for detecting, profiling, and monitoring certain sets of target microorganisms in a biological sample, using an assay developed to detect the presence of the microorganisms listed in Table 1 in a single sample preparation. An Applied Biosystems™ TaqMan™ Assay is a combination of an amplification primer pair and a fluorescently labeled probe designed to work in combination to amplify and detect a target nucleic acid, and the disclosed methods and compositions may include primer pairs and probes provided in the Applied Biosystems™ TaqMan™ Assays (assay IDs) listed in Table 1.

Panels of amplification primer pairs and corresponding detectably labeled probes are provided where each primer/probe combination is specific for a selected microorganism in Table 1. The microbe panel, independent of reaction, extraction, and/or other control targets, may include primer pairs specific for microorganisms listed in Table 1.

Panels of amplification primer pairs are disclosed herein for specific target genes listed in Table 1. In some embodiments, the gene panel, independent of reaction, extraction, and/or other control targets, includes primer pairs specific for at least five, at least ten, at least fifteen, or all of the genes listed in Table 1.

The disclosed methods may utilize panels of amplification primer pairs and a corresponding detectably labeled probe, where each primer/probe combination is specific for a microbial gene target listed in Table 1. In some embodiments, the microbial gene panel, independent of reaction, extraction, and/or other control targets, includes primer pairs specific for at least five, at least ten, at least fifteen, or all of the microbial genes listed in Table 1.

The type or presence of a microorganism in a biological sample can be identified or determined by analyzing a nucleic acid sample prepared from a biological sample. Once obtained or collected from a source, for example a subject or patient, a biological sample can be processed according to known methods to extract nucleic acids present in the sample. In other instances, a total nucleic acid sample can be prepared from the biological sample. In some instances, steps to enrich microorganisms in the biological sample may be taken prior to nucleic acid extraction. In some embodiments, the nucleic acid sample is amplified according to known methods, such as polymerase chain reaction (PCR). In some preferred embodiments, the PCR is a quantitative PCR (qPCR).

When applying quantitative methods to PCR-based technologies (e.g., qPCR), a fluorescent probe or other detectable label may be incorporated into the reaction to provide a means for determining the progress of the target amplification. In the case of a fluorescent probe, the reaction can be made to fluoresce in relative proportion to the quantity of nucleic acid product produced. As such, using PCR, assays for nucleotides sequences corresponding to the microorganism genes are the target sequences and can be used to determine the presence or absence of a microorganism in or the microbial profile of the biological sample.

The amplification reactions may occur on a support having reaction sites and each reaction site may include one pair of amplification primers. The amplification reactions may occur in reaction vessels and each reaction may include one pair of amplification primers. The reaction vessel may further include at least one target specific oligonucleotide probe, the probe being specific for nucleic acid portion amplified by the amplification primer pair present in individual a reaction site in or on the support. The reaction sites may be through-holes in a support plate and each through-hole may include one pair of amplification primers and at least one detectably-labeled probe as described herein. The primers or primers and probes may be dried in each reaction site of the reaction vessel. All of the reaction sites may in some cases reside on the same support or reaction vessel.

The support may provide a surface for the immobilization, attachment, or placement of amplification reagents (e.g., oligonucleotides, such as probes and/or primers), by any available method so that they are significantly or entirely prevented from diffusing freely or moving with respect to one another. The reagents can, for example, be placed in contact with the support, and optionally covalently or noncovalently attached or partially/completed embedded. Suitable supports are available commercially, and will be apparent to the skilled person. A solid support may be used for the methods, compositions and kits described herein. Such solid supports can include, but are not limited to, paper, nitrocellulose, myelin, glass, silica, nylon, plastics such as polyethylene, polypropylene or polystyrene, or other solid material. In addition, the support may be a gel constructed from such materials such as agarose, polyacrylamide, polysaccharide or proteins, which may themselves be overlaid on a further solid surface such as glass or metal, to provide mechanical strength, electrical conductivity or other desired physical property. The support may include a flat (planar) surface, or at least a structure in which the surface-bound oligonucleotides are attached in approximately the same plane. The solid support may in some cases be non-planar and may even be formed from discrete units, e.g. microbeads.

As used herein, the term “surface” means any generally two-dimensional structure on a solid support to which the desired oligonucleotide(s) is/are attached or immobilized.

The amplification reaction vessel can also contain other component reagents of the amplification reaction mixture as disclosed herein such as, for example, dNTPs (dATP, dCTP, dGTP, dTTP, and/or dUTP), one or more polymerases, a buffer(s), one or more salt(s), one or more detergent(s), one or more amplification inhibitor blocking agent(s), and/or one or more antifoam agent(s). Accordingly, semi-solid or solid supports may be provided with reaction sites or reaction chambers including an amplification primer pair together with an amplification reaction mixture or master mix. The primer pair and reaction mix combination in the reaction vessel or individual reaction site may be dried. The reaction mixture in the reaction site or reaction vessel may lyophilized and in some embodiments, can be applied to the reaction site or vessel as a dried deposit. Semi-solid or solid supports may be provided with reaction sites including an amplification primer pair and detectably labeled probe together with an amplification master mix to form a reaction mixture. The reaction mixture in the reaction site or reaction vessel may be dried. The reaction mix in the reaction site or reaction vessel may also be lyophilized.

Supports may be provided including a reaction site including a primer or a primer pair specific for at least five, at least ten, or at least fifteen of the genes listed in Table 1. Supports may be provided including a primer or a primer pair specific for all of the genes listed in Table 1, or supports may be provided including reaction sites wherein each reaction site includes a different primer or a primer pair specific for at least 2, 3, 4, 5, 6, 7, 8, 9, 10, 11, 12, 13, 14, 15, 16, or 17 of the genes listed in Table 1. The supports provided may further include a reaction site including a primer or a primer pair specific for at least 1, 2, 3, 4, 5, 6, 7, 8, 9, or 10 internal and/or external controls.

To more clearly and concisely describe and point out the subject matter of the present disclosure, certain definitions may be provided for specific terms, which are used in the following description and the appended claims. Throughout the specification, exemplification of specific terms should be considered as non-limiting examples.

The disclosure may refer to compositions for use as amplification controls and to methods for the use thereof in nucleic acid amplification processes. The provided control compositions and methods for their use provide users thereof tools and methods to monitor, evaluate, troubleshoot, and control nucleic acid amplification workflows. In some embodiments, the control nucleic acid molecules provided herein include at least two, at least five, at least then, at least fifteen or at least seventeen different target sequences.

The provided extraction and/or amplification control nucleic acid compositions can serve as positive and/or negative controls for workflows involving nucleic acid amplification and/or detection. The control compositions and methods for the use thereof provided herein may be used in conjunction with compositions and methods for amplification and characterization of select nucleic acids, or their respective cDNAs, derived from microorganisms in a biological sample. The control compositions and methods for use thereof as provided herein may be used in conjunction with compositions and methods for the detection of and/or evaluating microbiota profiles of select tissues and anatomical regions, for example, for detecting and/or monitoring bladder, urinary tract, and urogenital microbiome constituents and dynamics. As such, when used in conjunction with amplification reactions for a select set of assays for target nucleic acids or microorganisms in a biological sample, the control nucleic acid molecules used include the same target sequences to which the amplification and/or detection assays are directed. In some embodiments, the control nucleic acid molecules may include a subset of the target sequences to which the assays are directed. The control nucleic acid molecules may include additional target sequences to which additional reference or control assays are directed. The control nucleic acid molecules may include xeno target sequences (with no known homology to any organism) to which control assays are directed. In some embodiments, the control nucleic acid molecules may be a plasmid molecule comprising a plurality of target sequences, referred to herein as a superplasmid. In some embodiments, the superplasmid control nucleic acid molecule is linearized.

In some embodiments, provided herein are methods for amplifying target sequences in a control nucleic acid molecule sample including contacting at least some portion of the sample with target-specific primers and a polymerase under amplification conditions thereby producing at least one amplified target sequence. As described herein, the control nucleic acid molecule may include different target nucleic acid sequences. The disclosure provides methods for amplifying target sequences in a control nucleic acid molecule sample including contacting at least some portion of the sample with target-specific primers as disclosed herein and a polymerase under amplification conditions thereby producing at least one amplified target sequence, wherein each of the target-specific primers is provided in a multiplicity of separate reactions (e.g., as single-plex reactions). The disclosure provides methods for amplifying target sequences in a control nucleic acid molecule sample including contacting at least some portion of the sample with target-specific primers as disclosed herein and a polymerase under amplification conditions thereby producing at least one amplified target sequence, wherein the target-specific primers are provided in a single, combined reaction (e.g., as a multiplex reaction). The methods provided herein include contacting at least some portion of the sample with target-specific primer and probe sets (e.g., assays) as disclosed herein and a polymerase under amplification conditions thereby producing at least one amplified target sequence and detecting the presence of the at least one amplified target sequence. In some embodiments, each assay includes a forward primer and a reverse primer designed to specifically amplify a target sequence and a detectably labeled probe specific for the nucleic acid amplified by the forward and reverse primers (e.g., an amplicon).

The methods provided herein include subjecting a sample including a control nucleic acid molecule including different target sequences to multiple individual amplification reactions (i.e. single-plex reactions), each individual reaction performed with a pair of amplification primers designed to be specific for at least a portion of a target sequence in the control nucleic acid molecule and a detectably labeled probe specific of the target sequence amplified by the primers. The multiple individual amplification reactions can generate individual amplification products in separate reactions for each of the target sequences for which the amplification primers and detector probe are designed. Evaluation of the multiple amplification reactions can be arrived at by determining the presence or absence of, and/or by quantifying, the targeted amplification products from the individual (single-plex) amplification reactions.

The methods provided herein include subjecting a sample including a control nucleic acid molecule including different target sequences to an amplification reaction including a combination of primer pairs designed to be specific for target sequences in the control nucleic acid sample (i.e. multiplex reaction). The reaction is performed with at least two different pairs of amplification primers designed to be specific for at least two different target sequences in the control nucleic acid molecule and a detectably labeled probe specific for each of the different target sequences amplified by the different primers. The amplification reaction can generate multiple amplification products for each of the target sequences for which the combination of amplification primers and detector probes are designed. Evaluation of the multiple amplification reactions can be arrived at by determining the presence or absence of, and/or by quantifying, the targeted amplification products within the combined (multiplex) amplification reaction.

The detection assays of the compositions and methods provided herein may involve the use of oligonucleotide primers and a detectably labeled probe for amplification and detection of control nucleic acid specific target sequences. The target-specific primer and probe sets may be provided as part of a single-plex reaction, having a single set of primers and probes specific for a single nucleic acid target in a reaction. The target specific primer and probe sets may alternatively be provided as part of a multiplex reaction, having multiple sets of primers and probes specific for multiple and different nucleic acid targets within the same reaction.

Detection assays of the compositions and methods provided herein involve the use of oligonucleotide primers and a detectable nucleic acid binding moiety for amplification and detection of control nucleic acid specific target sequences. The target-specific primer and the detectable nucleic acid binding moiety are provided as part of a single-plex reaction. The target specific primer and the detectable nucleic acid binding moiety may alternatively be provided as part of a multiplex reaction. The detectable nucleic acid binding moiety may be a nucleic acid binding dye. The dye may be a double-stranded DNA binding dye. In some embodiments, the dye may be SYBR Green.

The compositions, methods and kits provided herein include additional amplification reactions and assays which are performed as additional reference or control reactions and assays. Without limitation, these additional reference or control reactions and assays can be used in relative quantification applications to assess the adequacy of the biological sample or the nucleic acid sample, to normalize microbial presence, and/or to detect the presence of amplification inhibitors in the biological or nucleic acid sample. Exemplary target nucleic acids for such additional reference or control assays include, without limitation, prokaryotic 16S rRNA gene sequence, human RNase P gene sequence, xeno nucleic acid (XNA) sequence and/or added exogenous nucleic acids.

The disclosure relates to compositions, methods, and kits for performing single-plex nucleic acid amplification reactions under the same assay conditions and/or at substantially the same time. The disclosure also relates to compositions, methods, and kits for performing multiplex nucleic acid amplification reactions under the same assay conditions and/or at substantially the same time.

In some embodiments, this disclosure relates to compositions, methods, and kits for detecting, monitoring, and evaluating extraction and/or amplification of control nucleic acid molecules including certain sets of target sequences derived from certain target microorganisms. In some embodiments, the control nucleic acid molecule is part of a plasmid including multiple target sequences (i.e., a multi-target plasmid or superplasmid). For example, in some embodiments as described herein, an amplification control nucleic acid molecule is developed to contain at least two different target sequences derived from the microorganisms and/or the microorganism genes listed in Table 1.

An Applied Biosystems™ TaqMan® Assay is a combination of an amplification primer pair (forward primer and reverse primer) and a fluorescently labeled probe designed to work in combination to amplify and detect a particular target nucleic acid. The compositions and methods disclosed herein may include microorganism-specific and/or gene-specific TaqMan® assays. The compositions and methods disclosed herein may include microorganism-specific TaqMan® assays directed to bladder, urogenital, and/or urinary tract microbiota. The compositions and methods disclosed herein include at least one of the primer pairs and probes provided in the Applied Biosystems™ TaqMan® Assays listed in Table 1. The methods may include at least two different sets of primer pairs and probes provided in the TaqMan® Assays listed in Table 1. The methods may include a select group or panel of the different sets of primer pairs and probes provided in the TaqMan® Assays listed in Table 1. The methods may include all of the different sets of primer pairs and probes provided in the TaqMan® Assays listed in Table 1.

In some embodiments, the use of TaqMan Assays as described herein provide a more sensitive and more accurate method for detection and identification of urinary tract microbiota when compared to data collected from culture-based methods. The traditional or routine (“gold standard”) approach for detecting and identifying UTI pathogens is to prepare urine cultures from a urine sample and monitor growth of microorganisms for 24 hours. Whether or not the culture shows microorganism growth is used to determine if a urine sample is positive (+growth) or negative (-growth) for a given uropathogen. This is referred to as a “culture-based” method for urinalysis.

Urine culture results are typically categorized on the basis of quantity and purity of microorganism growth. A commonly used criterion for defining bacterium and/or fungal growth is the presence of ≥10⁵ Colony Forming Units (CFU) per milliliter of urine. A UTI culture is typically deemed to be positive for a particular microorganism if there is a microorganism concentration of ≥10⁵ CFU/mL, while less than this concentration would be considered to be negative or as having “no significant growth.” In most cases, below a recognized threshold (≤10⁵ CFU/mL), the likelihood is that the organisms grown are contaminants, particularly if more than one type of organism is present. Above the threshold it is more probable that a true urinary tract infection is occurring. However, in some cases, there may be growth that is at a concentration of ≥10⁵ CFU per milliliter of urine, but there are too many different microorganisms (“mixed flora”) to accurately identify or distinguish the UTM. In these instances, culture results are also referred to as “negative” for having inconclusive data since growth of multiple (e.g., more than 2) organisms is also highly likely to be a contaminated specimen. Thus, a “true negative” culture is one that has no or low (≤10⁵cfu/mL) growth and a “true positive” culture is one that has significant growth (i.e., ≥10⁵ CFU/mL), while a “negative” sample having mixed flora “@≥10⁵ CFU/mL” is one where results are inconclusive or unidentifiable.

In some embodiments, the use of TaqMan Assays as described herein are at least 2× times more sensitive and/or accurate for the detection and/or identification of a UTI pathogen when compared to results obtained from a traditional culture-based method. In some embodiments, UTI TaqMan Assays may be at least 2×, 3×, 4×, 5× or 10× more sensitive and/or accurate when compared to results obtained from a traditional culture method. In some embodiments, UTI TaqMan Assays may be at least 20%, 30%, 40%, 50%, 60%, 70%, 80%, 90%, or 99% (and including all percentage numbers in between) more sensitive and/or accurate when compared results obtained from a traditional culture method. In some embodiments, the use of TaqMan Assays as described herein may identify the presence of at least 1 more, 2 more, 3 more, 5 more, 10 more, 15 more, or 17 more microorganisms (from those listed in Table 1) in a urine sample when compared to the number of microorganisms identified using a traditional culture-based method when testing the same urine sample.

In some embodiments, the PCR methods as described herein may provide positive and/or negative urinalysis results (for identifying the presence of a uropathogen), wherein the result is at least 80%, 90%, 95%, 96%, 97%, 98%, 99%, or 100% (and including all percentage numbers in between) concordant with the positive and/or negative urinalysis result obtained by Sanger Sequencing methods.

In some embodiments, the PCR methods as described herein may provide positive and/or negative urinalysis results (for identifying the presence of a uropathogen), wherein the result is at least 50%, 60%, 70%, 80%, 90%, 95%, 96%, 97%, 98%, 99%, or 100% (and including all percentage numbers in between) concordant with the positive and/or negative urinalysis result obtained by a traditional culture-based method.

Control nucleic acid molecules are provided which contain target sequences derived from selected genomic or transcriptomic sequences of different microorganisms. Target sequences may be derived from genomic or transcriptomic sequences from bacteria, fungi, protozoa, and/or viruses. Control nucleic acid molecules may include different target sequences derived from different genomic or transcriptomic sequences of different microorganisms from Table 1, for example, from 2 to about 17 different target sequences, or from 5 to 17 different target sequences, or from 10 to 17, or from 15 to 17 different target sequences. In some embodiments, control nucleic acid molecules are provided including at least 5, at least 10, at least 15, or at least 17 different target sequences derived from different microbial genes.

The different target sequences in the control nucleic acid molecules can vary in length. For example, in some embodiments, each of the different target sequences in the control nucleic acid molecule are from about 15 nucleotides to about 1000 nucleotides in length. In some embodiments, each of the different target sequences in the control nucleic acid molecule are from about 20 to about 800, about 25 to about 600, about 30 to about 500, about 40 to about 400, about 50 to about 300, about 56 to 105 nucleotides in length.

The control nucleic acid molecule may include a portion of the genomic or transcriptomic sequence or sequences which flank either side or both sides of the different target sequences. For example, the control nucleic acid molecule may include a portion of the 3′ flanking sequence, a portion of the 5′ flanking sequence or a portion of both the 3′ and 5′ flanking sequences for at least two of the different target sequences of the control nucleic acid molecule. The control nucleic acid molecule may include a portion of the 3′ flanking sequence, a portion of the 5′ flanking sequence or a portion of both the 3′ and 5′ flanking sequence corresponding to each of the different genomic or transcriptomic target sequences of the control nucleic acid molecule. The flanking sequence (if 3′ or 5′) or sequences (if 3′ and 5′) corresponding to the genomic or transcriptomic region or regions flanking each of the target sequences may be between 5 and 500 nucleotides in length. The flanking sequence(s) corresponding to the genomic or transcriptomic region or regions flanking each of the target sequences may be from about 10 to 400, about 15 to 200, about 20 to 100, or about 25 to 50 nucleotides in length. The control nucleic acid molecule may be target sequences derived from selected genomic or transcriptomic sequences of different microorganisms as well as their corresponding 3′, 5′, or 3′ and 5′ genomic or transcriptomic flanking sequences. The control nucleic acid molecule may include flanking sequences corresponding to only a portion of the different target sequences. For example, flanking sequence can be included in the target nucleic acid molecule for only 1, 2, 3, 4, 5, 6, 10, 15, 20, 25, or 30, etc. of the different target sequences. The different target sequences and only their corresponding 3′ flanking sequences may be included in the control nucleic acid molecule, and/or the different target sequences and only their corresponding 5′ flanking sequences may be included in the control nucleic acid molecule, and/or the different target sequences and both their corresponding 3′- and 5′-flanking sequences may be included in the control nucleic acid molecule. The different target sequences and a combination of either the corresponding 3′ flanking, 5′ flanking, 3′ and 5′ flanking or no flanking sequences for each target sequence may be in the control nucleic acid molecule. The control nucleic acid molecule may in some cases not include any corresponding genomic or transcriptomic flanking sequences.

The control nucleic acid molecule including the different target sequences (with or without flanking sequences included) can vary in length. The length of the entire control nucleic acid molecule may be between 0.5 kb to 50 kb in length. The entire control nucleic acid molecule may be from about 1 kb to 20 kb, about 2 kb to 15 kb, about 3 kb to 10 kb in length, or about 4 kb to 5 kb in length. A portion of or the entire sequence of the control nucleic acid molecule may be inserted into or contained within a nucleic acid construct including, without limitation, a vector, plasmid, or virus.

When applying quantitative methods to polymerase chain reaction (PCR)-based technologies (e.g., qPCR), a fluorescent probe or other detectable label may be incorporated into the reaction to provide a means for determining the progress of the target amplification. Through the use of the fluorescent probe or other detectable label, such as a nucleic acid binding moiety, the reaction can be made to fluoresce in relative proportion to the quantity of nucleic acid product produced. As such, when using PCR, assays for nucleotide sequences corresponding to the control target sequences can be used to determine the efficacy of the amplification reaction and/or extraction process for the control nucleic acid sample. The fluorescent probe can be used in a sequence-specific manner for detection of specific nucleic acids. The detectable label can be used in a non-sequence-specific manner for general detection of nucleic acids.

The amplification reactions occur on a support having reaction sites and each reaction site may include one pair of amplification primers. The amplification reactions occur in reaction vessels and each reaction may include one pair of amplification primers. The reaction vessel further may include at least one target specific oligonucleotide probe, the probe being specific for a portion of the nucleic acid amplified by the amplification primer pair present in the reaction vessel. As noted, the reaction vessels can comprise individual reaction sites. In some embodiments, the reaction sites can be through-holes in a support plate and each through-hole may include one pair of amplification primers and at least one detectably-labeled probe as described herein. The primers and probes may be dried in each reaction site or reaction vessel prior to contact with the control nucleic acid sample comprising a control nucleic acid molecule.

The amplification reaction vessel can also contain other component reagents of the amplification reaction mixture such as, for example, dNTPs (dATP, dCTP, dGTP, dTTP and/or dUTP), polymerase, buffer(s), at least one salt(s), at least one detergent(s), at least one amplification inhibitor blocking agent(s), and/or at least one antifoam agent(s). Accordingly, semi-solid or solid supports may be provided with reaction sites including a control nucleic acid molecule and an amplification primer pair together with an amplification master mix. The primer pair and master mix combination in the reaction site or reaction vessel may be dried prior to addition of a control nucleic acid sample. Semi-solid or solid supports may be provided with reaction sites including a control nucleic acid molecule, an amplification primer pair and detectably labeled probe together with an amplification master mix. The primer pair, probe, and master mix combination in the reaction site or reaction vessel may be dried prior to addition of a control nucleic acid sample.

In some embodiments, the nucleic acid sample may be DNA or RNA, such as genomic DNA (gDNA). The nucleic acid sample may comprise single-stranded or double-stranded nucleic acid molecules. The nucleic acid sample may be obtained from any source, including for example cultured cells or a biological test sample. It will be appreciated that nucleic acid sample may be isolated from a biological source using any of a variety of procedures known in the art, for example, MagMAX™ DNA Multi-Sample Ultra Kit (Applied Biosystems, Thermo Fisher Scientific), the MagMAX™ Express-96 Magnetic Particle Processor and the KingFisher™ Flex Magnetic Particle Processor (Thermo Fisher Scientific), the ABI Prism™ 6100 Nucleic Acid PrepStation and the ABI Prism™ 6700 Automated Nucleic Acid Workstation (Applied Biosystems, Thermo Fisher Scientific), and the like. It will be appreciated that nucleic acids sample may be fragmented prior to analysis, including the use of such procedures as mechanical force, restriction endonuclease cleavage, or any method known in the art. In some embodiments, the nucleic acids sample may be in a crude lysate when amplified and/or analyzed.

As used in this specification, the words “a” or “an” means at least one, unless specifically stated otherwise. In this specification, the use of the singular includes the plural unless specifically stated otherwise. For example, but not as a limitation, “a target nucleic acid” means that more than one target nucleic acid can be present; for example, one or more copies of a particular target nucleic acid species, as well as two or more different species of target nucleic acid. The term “and/or” means that the terms before and after the slash can be taken together or separately. For illustration purposes, but not as a limitation, “X and/or Y” can mean “X” or “Y” or “X” and “Y”.

It will be appreciated that there is an implied “about” prior to the temperatures, concentrations, times, etc. discussed in the present disclosure, such that slight and insubstantial deviations are within the scope of the present teachings herein. Also, the use of “include”, “includes”, “including”, “contain”, “may include”, “including”, “include”, “includes”, and “including” are not intended to be limiting. It is to be understood that both the foregoing general description and detailed description are exemplary and explanatory only and are not restrictive of the teachings.

Unless specifically noted in this specification, embodiments in this specification that recite “including” various components are also contemplated as “consisting of” or “consisting essentially of” the recited components; embodiments in the specification that recite “consisting of” various components are also contemplated as “including” or “consisting essentially of” the recited components; and embodiments in the specification that recite “consisting essentially of” various components are also contemplated as “consisting of” or “including” the recited components (this interchangeability does not apply to the use of these terms in the claims).

As used herein, the terms “amplification”, “nucleic acid amplification”, or “amplifying” refer to the production of multiple copies of a nucleic acid template, or the production of multiple nucleic acid sequence copies that are complementary to the nucleic acid template. The terms (including the term “polymerizing”) may also refer to extending a nucleic acid template (e.g., by polymerization). The amplification reaction may be a polymerase-mediated extension reaction such as, for example, a polymerase chain reaction (PCR). However, any of the known amplification reactions may be suitable for use as described herein. The term “amplifying” that typically refers to an “exponential” increase in target nucleic acid may be used herein to describe both linear and exponential increases in the numbers of a select target sequence of nucleic acid.

The terms “amplicon” and “amplification product” as used herein generally refer to the product of an amplification reaction. An amplicon may be double-stranded or single-stranded, and may include the separated component strands obtained by denaturing a double-stranded amplification product. In certain embodiments, the amplicon of one amplification cycle can serve as a template in a subsequent amplification cycle.

The terms “annealing” and “hybridizing”, including, without limitation, variations of the root words “hybridize” and “anneal”, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which primers and probes anneal to complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968).

In general, whether such annealing takes place is influenced by, among other things, the length of the complementary portions of the complementary portions of the primers and their corresponding binding sites in the target flanking sequences and/or amplicons, or the corresponding complementary portions of a reporter probe and its binding site; the pH; the temperature; the presence of mono- and divalent cations; the proportion of G and C nucleotides in the hybridizing region; the viscosity of the medium; and the presence of denaturants. Such variables influence the time required for hybridization. Thus, the preferred annealing conditions will depend upon the particular application. Such conditions, however, can be routinely determined by persons of ordinary skill in the art, without undue experimentation. Preferably, annealing conditions are selected to allow the primers and/or probes to selectively hybridize with a complementary sequence in the corresponding target flanking sequence or amplicon, but not hybridize to any significant degree to different target nucleic acids or non-target sequences in the reaction composition at the second reaction temperature.

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The terms “denaturing” and “denaturation” as used herein refer to any process in which a double-stranded polynucleotide, including without limitation, a genomic DNA (gDNA) fragment including at least one target nucleic acid, a double-stranded amplicon, or a polynucleotide including at least one double-stranded segment is converted to two single-stranded polynucleotides or to a single-stranded or substantially single-stranded polynucleotide, as appropriate. Denaturing a double-stranded polynucleotide includes, without limitation, a variety of thermal and chemical techniques which render a double-stranded nucleic acid single-stranded or substantially single-stranded, for example but not limited to, releasing the two individual single-stranded components of a double-stranded polynucleotide or a duplex including two oligonucleotides. Those in the art will appreciate that the denaturing technique employed is generally not limiting unless it substantially interferes with a subsequent annealing or enzymatic step of an amplification reaction, or in certain methods, the detection of a fluorescent signal.

As used herein, the term “Tm” is used in reference to melting temperature. The melting temperature is the temperature at which a population of double-stranded nucleic acid molecules becomes half dissociated into single strands.

The term “minor groove binder” as used herein refers to a small molecule that fits into the minor groove of double-stranded DNA, sometimes in a sequence specific manner. Generally, minor groove binders are long, flat molecules that can adopt a crescent-like shape and thus, fit snugly into the minor groove of a double helix, often displacing water. Minor groove binding molecules typically include several aromatic rings connected by bonds with torsional freedom, for example, but not limited to, furan, benzene, or pyrrole rings.

The term “end-point” measurement refers to a method where data collection occurs only once the reaction has been stopped.

The terms “real-time” and “real-time continuous” are interchangeable and refer to a method where data collection occurs through periodic monitoring during the course of the polymerization reaction. Thus, the methods combine amplification and detection into a single step.

As used herein the terms “Ct” and “cycle threshold” refer to the time at which fluorescence intensity is greater than background fluorescence. They are characterized by the point in time (or PCR cycle) where the target amplification is first detected. Consequently, the greater the quantity of target DNA in the starting material, the faster a significant increase in fluorescent signal will appear, yielding a lower Ct.

As used herein, the term “primer” and its derivatives refer generally to any polynucleotide that can hybridize to a target nucleic acid. The primer can also serve to prime nucleic acid synthesis. The primer is a synthetically or biologically produced single-stranded oligonucleotide that is extended by covalent bonding of nucleotide monomers during amplification or polymerization of a nucleic acid molecule. Nucleic acid amplification often is based on nucleic acid synthesis by a nucleic acid polymerase or reverse transcriptase. Many such polymerases or reverse transcriptases require the presence of a primer that may be extended to initiate such nucleic acid synthesis. A primer is typically about 11 bases to about 35 bases in length, although shorter or longer primers may be used depending on the need. In certain embodiments, a primer is 17 bases or longer. In certain embodiments, a primer is about 17 bases to about 25 bases in length. A primer may include standard, non-standard, derivatized and modified nucleotides. As will be appreciated by those skilled in the art, the oligonucleotides disclosed herein may be used as one or more primers in various extension, synthesis, or amplification reactions.

Typically, a PCR reaction employs a pair of amplification primers including an “upstream” or “forward” primer and a “downstream” or “reverse” primer, which delimit a region of the RNA or DNA to be amplified. A first primer and a second primer may be either a forward or reverse primer and are used interchangeably herein and are not to be limiting.

The terms “complementarity” and “complementary” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementarity refers to the situation in which each nucleotide unit of one polynucleotide strand or region can hydrogen bond with each nucleotide unit of a second polynucleotide strand or region. “Less than perfect complementarity” refers to the situation in which some, but not all, nucleotide units of two strands or two units can hydrogen bond with each other.

As used herein, the term “reverse complement” refers to a sequence that will anneal/base pair or substantially anneal/base pair to a second oligonucleotide according to the rules defined by Watson-Crick base pairing and the antiparallel nature of the DNA-DNA, RNA-RNA, and RNA-DNA double helices. Thus, as an example, the reverse complement of the RNA sequence 5′-AAUUUGC would be 5′-GCAAAUU. Alternative base pairing schemes, including but not limited to G-U pairing, can also be included in reverse complements.

As used herein, the term “probe” refers to synthetic or biologically produced nucleic acids (DNA or RNA) which, by design or selection, contain specific nucleotide sequences that allow them to hybridize, under defined stringencies, specifically (i.e., preferentially) to target nucleic acid sequences.

In some embodiments, the amplification control nucleic acids provided herein are used in conjunction with methods, compositions and kits for amplifying, detecting, profiling and/or monitoring target nucleic acids in a nucleic acid sample from a biological sample.

“Biological sample” or “test sample” includes cells, sections of tissues such as biopsy and autopsy samples, and frozen sections taken for histologic purposes, as well as fluid or secretion specimens that arise from cells or tissues. Such samples include biopsies, blood and blood fractions or products (e.g., serum, platelets, red blood cells, and the like), lymph, bone marrow, sputum, bronchoalveolar lavage, amniotic fluid, hair, skin, cultured cells, e.g., primary cultures, explants, and transformed cells, stool, urine, etc. In some embodiments, where the sample is derived from urine, the sample can be collected by urine voiding, through the use of a catheter or by suprapubic aspiration. Prior to target nucleic acid preparation, biological samples may be fresh, frozen or formalin- or paraformalin-fixed paraffin-embedded tissue (FFPE). A “biopsy” refers to the process of removing a tissue sample for diagnostic or prognostic evaluation, and to the tissue specimen itself. The biopsy technique applied will depend on the tissue type to be evaluated (e.g., skin, mucosa, etc.), the size and type of the tissue sample, among other factors. Representative biopsy techniques include, but are not limited to, excisional biopsy, incisional biopsy, needle biopsy, and surgical biopsy

In some embodiments, the amplification control nucleic acids provided herein are used in conjunction with methods, compositions and kits amplifying, detecting, profiling and/or monitoring nucleic acids from certain sets of target microorganisms in a biological or test sample. In some embodiments, a biological or test sample is from the urinary tract (e.g., urogenital mucosa, urethra, urogenital area) and includes cells, tissue and/or fluids (e.g., urinary tract secretions, urinary fluids, and urogenital secretions) from these anatomical sites.

Urine samples may be collected using any urine collection device, container or instrument readily known to those of skill in the art. In some embodiments, for example, urine can be collected using a BD Vacutainer® urine collection cup; a BD Vacutainer® urinalysis preservative tube; a BD Vacutainer® Plus C&S preservative tube; Hologics® Aptima Urine Specimen Transport Tubes, and the like. The urine specimen can be collected by any means known to those of skill in the art. For example, urine can be collected by urine voiding, through the use of a catheter, or by suprapubic aspiration. Collection systems, reagents, and media compatible with, for example, urethral or urogenital biological samples are known in the art and contemplated for use with the methods, compositions and kits as disclosed herein.

It will be appreciated that nucleic acids may be isolated from biological samples using any of a variety of procedures known in the art, for example, using a MagMAX™ DNA Multi-Sample Ultra Kit (Applied Biosystems, Thermo Fisher Scientific), a MagMAX™ Express-96 Magnetic Particle Processor (Thermo Fisher Scientific) , a KingFisher™ Flex Magnetic Particle Processor (Thermo Fisher Scientific), a PureLink™ Microbiome DNA Purification Kit (Invitrogen, Thermo Fisher Scientific), a RecoverAll™ Total Nucleic Acid Isolation Kit for FFPE(Ambion™, Thermo Fisher Scientific), a PureLink™ FFPE RNA Isolation Kit (Ambion™, Thermo Fisher Scientific), an ABI Prism™ 6100 Nucleic Acid PrepStation and an ABI Prism™ 6700 Automated Nucleic Acid Workstation (Applied Biosystems, Thermo Fisher Scientific), and the like. It will be appreciated that target nucleic acids from the biological samples may be cut or sheared prior to analysis, including the use of such procedures as mechanical force, sonication, restriction endonuclease cleavage, or any method known in the art.

As used herein, the term “template” is interchangeable with “target molecule” or “target nucleic acid” and refers to a double-stranded or single-stranded nucleic acid molecule which is to be amplified, copied or extended, synthesized, or sequenced. In the case of a double-stranded DNA molecule, denaturation of its strands to form a first and a second strand is performed to amplify, sequence, or synthesize these molecules. Target nucleic acids can include the nucleic acid sequences to which primers useful in the amplification or synthesis reaction can hybridize prior to extension by a polymerase. A primer, complementary to a portion of a template is hybridized under appropriate conditions and the polymerase (e.g., DNA polymerase or reverse transcriptase) may then synthesize a nucleic acid molecule complementary to the template or a portion thereof. The newly synthesized molecule, according to the present disclosure, may be equal or shorter in length than the original template. Mismatch incorporation during the synthesis or extension of the newly synthesized molecule may result in one or a number of mismatched base pairs. Thus, the synthesized molecule need not be exactly complementary to the template. The template may be an RNA molecule, a DNA molecule, or a DNA/RNA hybrid molecule. A newly synthesized molecule may serve as a template for subsequent nucleic acid synthesis or amplification.

The target nucleic acid may be a nucleic acid (e.g., DNA or RNA), genomic DNA (gDNA), cell-free DNA, circulating DNA, cDNA, messenger RNA (mRNA), transfer RNA (tRNA), small interfering RNA (siRNA), microRNA (miRNA), or other mature small RNA, and may include nucleic acid analogs or other nucleic acid mimics. The target may be methylated, non-methylated, or both. The target may be bisulfate-treated and non-methylated cytosines converted to uracil. Further, it will be appreciated that “target nucleic acid” may refer to the target nucleic acid itself, as well as surrogates thereof, for example, amplification products and native sequences.

The target nucleic acid may be obtained from any source, and may include any number of different compositional components. The target molecules of the present teachings may be derived from any number of sources, including without limitation, viruses, archae, protists, prokaryotes and eukaryotes, for example, from a biological sample obtained from a eukaryotic organism, most preferably a mammal such as a primate e.g., chimpanzee or human. It will be appreciated that target nucleic acids may be isolated from biological samples using any of a variety of procedures known in the art, for example, MagMAX™ DNA Multi-Sample Ultra Kit (Applied Biosystems, Thermo Fisher Scientific), the MagMAX™ Express-96 Magnetic Particle Processor and the KingFisher™ Flex Magnetic Particle Processor (Thermo Fisher Scientific), a RecoverAll™ Total Nucleic Acid Isolation Kit for FFPE and PureLink™ FFPE RNA Isolation Kit (Ambion™, Thermo Fisher Scientific), the ABI Prism™ 6100 Nucleic Acid PrepStation and the ABI Prism™ 6700 Automated Nucleic Acid Workstation (Applied Biosystems, Thermo Fisher Scientific), and the like. It will be appreciated that target nucleic acids may be cut or sheared prior to analysis, including the use of such procedures as mechanical force, sonication, restriction endonuclease cleavage, or any method known in the art. In general, the target nucleic acids of the present teachings will be single-stranded, though in some embodiments the target nucleic acids may be double-stranded, and a single-strand may result from denaturation.

The term “incorporating” as used herein, means becoming a part of a DNA or RNA molecule or primer.

The term “nucleic acid binding moiety” as used herein refers to a molecule which has an affinity for binding nucleic acid molecules such as DNA, RNA or DNA/RNA hybrids.

The term “nucleic acid binding dye” as used herein refers to a fluorescent molecule that is specific for a double-stranded polynucleotide or that at least shows a substantially greater fluorescent enhancement when associated with double-stranded polynucleotides than with a single stranded polynucleotide. Typically, nucleic acid binding dye molecules associate with double-stranded segments of polynucleotides by intercalating between the base pairs of the double-stranded segment, but binding in the major or minor grooves of the double-stranded segment, or both. Non-limiting examples of nucleic acid binding dyes include ethidium bromide, DAPI, Hoechst derivatives including without limitation Hoechst 33258 and Hoechst 33342, intercalators including a lanthanide chelate (for example, but not limited to, a naphthalene diimide derivative carrying two fluorescent tetradentate β-diketone-Eu3+ chelates (NDI-(BHHCT-Eu3+)2), see e.g., Nojima et al., Nucl. Acids Res. Suppl. No. 1 105 (2001), and certain asymmetrical cyanine dyes such as SYBR® Green and PicoGreen®.

As used herein, the terms “polynucleotide,” “oligonucleotide,” and “nucleic acid” are used interchangeably and refer to single-stranded and double-stranded polymers of nucleotide monomers, including without limitation, 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+, and the like. A polynucleotide may be composed entirely of deoxyribonucleotides, entirely of ribonucleotides, or chimeric mixtures thereof and may include nucleotide analogs. The nucleotide monomer units may include any of the nucleotides described herein, including, but not limited to, nucleotides and/or nucleotide analogs. Polynucleotides typically range in size from a few monomeric units, e.g., 5-40 when they are sometimes referred to in the art as oligonucleotides, to several thousands of monomeric nucleotide units. Unless denoted otherwise, whenever a polynucleotide sequence is represented, it will be understood that the nucleotides are in the 5′-to-3′ order from left to right and that “A” denotes deoxyadenosine, “C” denotes deoxycytosine, “G” denotes deoxyguanosine, “T” denotes deoxythymidine, and “U” denotes deoxyuridine, unless otherwise noted.

The term “nucleotide” refers to a phosphate ester of a nucleoside, e.g., triphosphate esters, wherein the most common site of esterification is the hydroxyl group attached at the C-5 position of the pentose.

The term “nucleoside” refers to a compound consisting of a purine, deazapurine, or pyrimidine nucleoside base, e.g., adenine, guanine, cytosine, uracil, thymine, deazaadenine, deazaguanosine, and the like, linked to a pentose at the 1′ position, including 2′-deoxy and 2′-hydroxyl forms. When the nucleoside base is purine or 7-deazapurine, the pentose is attached to the nucleobase at the 9- position of the purine or deazapurine, and when the nucleobase is purimidine, the pentose is attached to the nucleobase at the 1-position of the pyrimidine.

The term “analog” includes synthetic analogs having modified base moieties, modified sugar moieties, and/or modified phosphate ester moieties. Phosphate analogs generally include analogs of phosphate wherein the phosphorous atom is in the +5 oxidation state and one or more of the oxygen atoms is replaced with a non-oxygen moiety, e.g. sulfur. Exemplary phosphate analogs include: phosphorothioate, phosphorodithioate, phosphoroselenoate, phosphorodiselenoate, phosphoroanilothioate, phosphoranilidate, phosphoramidate, boronophosphates, including associated counterions, e.g., H+, NH4+, Na+. Exemplary base analogs include: 2,6-diaminopurine, hypoxanthine, pseudouridine, C-5-propyne, isocytosine, isoguanine, 2-thiopyrimidine. Exemplary sugar analogs include: 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, and bromo.

As used herein, the term “superplasmid” refers to a plasmid (DNA molecule) including an insert fragment that includes multiple, at least two, at least three, at least five, at least ten, at least fifteen, at least seventeen, nucleic acid target sequences of interest. The nucleic acid target sequences can be genomic or transcriptomic sequences. The nucleic acid target sequences can be xeno nucleic acids (XNA). The nucleic acid target sequences can be any combination of genomic, transcriptomic or xeno nucleic acid sequences.

As used herein, the term “reaction vessel” generally refers to any container, chamber, device, card, plate, chip, array or assembly, in which a reaction can occur in accordance with the present teachings. In some embodiments, a reaction vessel may be a microtube, for example, but not limited to, a 0.2 mL or a 0.5 mL reaction tube such as a MicroAmp™ Optical tube (Life Technologies Corp., Carlsbad, Calif.) or a micro-centrifuge tube, or other containers of the sort in common practice in molecular biology laboratories. In some embodiments, a reaction vessel can comprise individual reaction sites, For example, a reaction site can include a well of a multi-well plate (such as a 48-, 96-, or 384-well microtiter plate), a spot on a glass slide, a well in a TaqMan™ Array Card or a channel or chamber of a microfluidics device, including without limitation a TaqMan™ Low Density Array, or a through-hole of a TaqMan™ OpenArray™ Real-Time PCR plate (Applied Biosystems, Thermo Fisher Scientific). For example, but not as a limitation, multiple reaction sites can reside on the same support or within the same reaction vessel. In some embodiments, an OpenArray™ Plate, for example, is a reaction plate with 3072 through-holes or different reaction sites. Each such through-hole in such a plate may contain a single TaqMan™ assay. In some embodiments, lab-on-a-chip-like devices are available, for example, from Caliper or Fluidigm. It will be recognized that a variety of reaction vessels, some of which comprise multiple reaction sites, are commercially available or can be designed for use in the context of the present teachings.

The term “reporter group” is used in a broad sense herein and refers to any identifiable or detectable tag, label, or moiety.

The term “thermostable” when used in reference to an enzyme, refers to an enzyme (such as a polypeptide having nucleic acid polymerase activity) that is resistant to inactivation by heat. A “thermostable” enzyme is in contrast to a “thermolabile” polymerase, which can be inactivated by heat treatment. Thermolabile proteins can be inactivated at physiological temperatures, and can be categorized as mesothermostable (inactivation at about 45° C. to about 65° C.), and thermostable (inactivation at greater than about 65° C.). For example, the activities of the thermolabile T5 and T7 DNA polymerases can be totally inactivated by exposing the enzymes to a temperature of about 90° C. for about 30 seconds. A thermostable polymerase activity is more resistant to heat inactivation than a thermolabile polymerase. However, a thermostable polymerase does not mean to refer to an enzyme that is completely resistant to heat inactivation; thus heat treatment may reduce the polymerase activity to some extent, for example, especially if exposed to heat over a long period of time and/or for a repeated number of instances. A thermostable polymerase typically will also have a higher optimum temperature than thermolabile DNA polymerases.

The term “working concentration” refers to the concentration of a reagent that is at or near the optimal concentration used in a solution to perform a particular function (such as synthesis or digestion of a nucleic acid molecule). The working concentration of a reagent is also described equivalently as a “1× concentration” or a “1× solution” (if the reagent is in solution) of the reagent. Accordingly, higher concentrations of the reagent may also be described based on the working concentration; for example, a “2× concentration” or a “2× solution” of a reagent is defined as a concentration or solution that is twice as high as the working concentration of the reagent; a “5× concentration” or a “5× solution” is five times as high as the working concentration, and so on.

The term “reaction mixture” and/or “master mix” may refer to an composition including the various (some or all) reagents and/or components used to synthesize or amplify a target nucleic acid. Such reactions may also be performed using solid supports or semi-solid supports (e.g., an array). The reactions may also be performed in single or multiplex format as desired by the user. These reactions typically include enzymes, aqueous buffers, salts, amplification primers, target nucleic acid, and nucleoside triphosphates. The amplification reaction mixture and/or master mix may include one or more of, for example, a buffer (e.g., Tris), one or more salts (e.g., MgCl2, KC1), glycerol, dNTPs (dA, dT, dG, dC, dU), recombinant BSA (bovine serum albumin), a dye (e.g., ROX passive reference dye or a tracking dye), one or more detergents (e.g., Triton X-100, Nonidet P-40, Tween 20, Brij-58), polyethylene glycol (PEG), polyvinyl pyrrolidone (PVP), gelatin (e.g., fish or bovine source) and/or an antifoam agent. Depending upon the context, the mixture can be either a complete or incomplete amplification reaction mixture. In some embodiments, the master mix does not include amplification primers prior to use in an amplification reaction. In some other embodiments, the master mix does not include target nucleic acid prior to use in an amplification reaction. In some embodiments, an amplification master mix is mixed with a target nucleic acid sample prior to contact with amplification primers. In some other embodiments, an amplification master mix is mixed with amplification primers prior to contact with a target nucleic acid sample.

In some embodiments, the amplification reaction mixture includes amplification primers and a master mix. In some other embodiments, the amplification reaction mixture includes amplification primers, a detectably labeled probe, and a master mix. In some embodiments, the reaction mixture of amplification primers and master mix or amplification primers, probe and master mix are dried in a storage vessel or reaction vessel. In some other embodiments, the reaction mixture of amplification primers and master mix or amplification primers, probe and master mix are lyophilized in a storage vessel or reaction vessel.

The disclosure relates to the amplification of multiple target-specific sequences from a single nucleic acid source or sample. For example, in some embodiments that single nucleic acid sample can include RNA (microbial or otherwise) and in other embodiments, that single nucleic acid sample can include genomic DNA (including microbial genomic DNA). In some embodiments, nucleic acid molecules from at least one other source (e.g., an external control nucleic acid) are combined with the single nucleic acid sample in a reaction mixture prior to the target-specific amplification. It is envisioned that the sample can be from a single individual. The target-specific primers and primer pairs are target-specific sequences that can amplify specific regions of a nucleic acid molecule, for example, a control nucleic acid molecule. The target-specific primers can prime reverse transcription of an RNA to generate a target-specific cDNA. The target-specific primers can amplify microbial DNA, such as bacterial DNA, fungal (e.g., yeast) DNA, protozoa DNA, or viral DNA.

In one embodiment, a sample including one or more target sequences can be amplified using any one or more of the target-specific primers disclosed herein. In another embodiment, amplified target sequences obtained using the methods and associated compositions and kits disclosed herein, can be coupled to a downstream process, such as but not limited to, nucleic acid sequencing. For example, once the nucleic acid sequence of an amplified target sequence is known, the nucleic acid sequence can be compared to one or more reference samples. The output from the amplification procedure can be optionally analyzed for example by nucleic acid sequencing to determine if the expected amplification product based on the target-specific primers is present in the amplification output. In some embodiments, amplicons generated by the selective amplification can be cloned prior to sequencing or the amplicons can be sequenced directly without cloning. It will be understood by those of skill in the art, that the amplicons can be sequenced using any suitable DNA sequencing platform. For example, the amplicons can be sequenced using an Ion Personal Genome Machine™ (PGM™) System or an Ion Proton™ System (Thermo Fisher Scientific) or any other commercially available platform or methodology known to those having skill in the art.

In some embodiments the length of the amplicon that is produced can be modulated through the use of the selected primer pair. In some aspects, each primer of the set (e.g., the forward primer and the reverse primer) can be configured to specifically hybridize to all or a portion of a different region of a target nucleic acid, such that amplifying the target nucleic acid with the selected primer pair results in an amplicon having a specific size. The different regions of the target nucleic acid that each primer hybridizes to can be separated by at least 10 nucleotides, at least 20 nucleotides, at least 50 nucleotides, at least 100 nucleotides, at least 250 nucleotides, at least 500 nucleotides, at least 750 nucleotides, etc. Thus, in some embodiments, the selected primer set can produce an amplicon that is at least 10 nucleotides long, at least 20 nucleotides long, at least 50 nucleotides long, at least 100 nucleotides long, at least 250 nucleotides long, at least 500 nucleotides long, at least 750 nucleotides long, etc. In some embodiments, the selected primer pair produces an amplicon that is less than 500 bases in length, less than 300 bases in length, less than 200 bases in length, or less than 100 bases in length. In some embodiments, the amplicon that is produced is between 20 to 500 nucleotides long. For example, the amplicon can be 20 nucleotides long, 50 nucleotides long, 100 nucleotides long, 200 nucleotides long, 300 nucleotides long, 400 nucleotides long, 500 nucleotides long, or any length in between (e.g., any length between and including 20 to 500 nucleotides long). Systems and methods for designing and selecting sets of amplification primers to give a desired amplicon size, for use according to the methods, compositions and kits described herein, are known to those of skill in the art. See, for example, WO2013134341 A1 and https://www.ncbi.nlm.nih.gov/tools/primer-blast/. Those of skill in the art can also readily determine standard methods for determining amplicon length. For example, in some embodiments, a DNA size marker can be used to demonstrate relative amplicon sizes.

In one embodiment, a nucleic acid control including one or more target sequences can be amplified using any one or more of the target-specific primers disclosed herein. In another embodiment, amplified target sequences obtained using the methods and associated compositions and kits disclosed herein, can be coupled to a downstream process, such as but not limited to, nucleic acid sequencing. For example, once the nucleic acid sequence of an amplified target sequence is known, the nucleic acid sequence can be compared to one or more reference samples. The output from the amplification procedure can be optionally analyzed for example by nucleic acid sequencing to determine if the expected amplification product based on the target-specific primers is present in the amplification output. In some embodiments, amplicons generated by the selective amplification can be cloned prior to sequencing or the amplicons can be sequenced directly without cloning. The amplicons can be sequenced using any suitable DNA sequencing platform. For example, the amplicons can be sequenced using an Ion Personal Genome Machine™ (PGM™) System or an Ion Proton™ System (Thermo Fisher Scientific) or any other commercially available instrumentation.

The method used to amplify the target nucleic acid may be any available to one of skill in the art. Any in vitro means for multiplying the copies of a target sequence of nucleic acid may be utilized. These include linear, logarithmic, real-time, quantitative, end-point and/or any other amplification method. While this disclosure may generally discuss using a polymerase chain reaction (PCR or qPCR) as the nucleic acid amplification reaction, it is expected that the compositions, methods and kits describe herein should be effective with other types of nucleic acid amplification reactions, including both polymerase-mediated amplification reactions (such as helicase-dependent amplification (HDA), recombinase-polymerase amplification (RPA), and rolling circle amplification (RCA)), as well as ligase-mediated amplification reactions (such as ligase detection reaction (LDR), ligase chain reaction (LCR), and gap-versions of each), and combinations of nucleic acid amplification reactions such as LDR and PCR (see, for example, U.S. Pat. No. 6,797,470). Exemplary methods for nucleic acid synthesis include polymerase chain reaction (PCR; see, e.g., U.S. Pat. Nos. 4,683,202; 4,683,195; 4,965,188; and/or 5,035,996), isothermal procedures (using one or more RNA polymerases (see, e.g., PCT Pub. No. WO 2006/081222), strand displacement (see, e.g., U.S. Pat. No. RE39007E), partial destruction of primer molecules (see, e.g., PCT Pub. No. WO 2006/087574)), ligase chain reaction (LCR) (see, e.g., Wu, et al., Genomics 4: 560-569 (1990)), and/or Barany, et al. Proc. Natl. Acad. Sci. USA 88:189-193 (1991)), Qβ RNA replicase systems (see, e.g., PCT Pub. No. WO 1994/016108), RNA transcription-based systems (e.g., TAS, 3SR), rolling circle amplification (RCA) (see, e.g., U.S. Pat. No. 5,854,033; U.S. Pat. Pub. No. 2004/265897; Lizardi et al. Nat. Genet. 19: 225-232 (1998); and/or Bailer et al. Nucleic Acid Res., 26: 5073-5078 (1998)), and strand displacement amplification (SDA) (Little, et al. Clin. Chem. 45:777-784 (1999)), among others. These systems, along with the many other systems available to the skilled artisan, may be suitable for use in polymerizing and/or amplifying target nucleic acids for use as described herein.

In certain embodiments, amplification techniques include at least one cycle of amplification, for example, but not limited to, the steps of: denaturing a double-stranded nucleic acid to separate the component strands; hybridizing a primer or set of primers to a target sequence or primer-binding site(s) of an amplicon (or complements of either, as appropriate); and synthesizing a strand of nucleotides in a template-dependent manner using a DNA polymerase or a polypeptide having DNA polymerase activity. The cycle may or may not be repeated. In certain embodiments, a cycle of amplification includes a multiplicity of amplification cycles, for example, but not limited to 20 cycles, 25 cycles, 30 cycles, 35 cycles, 40 cycles, 45 cycles or more than 45 cycles of amplification.

In some embodiments, amplifying includes thermal cycling using an instrument, for example, but not limited to, a GeneAmp® PCR System 9700, 9600, 2700 or 2400 thermocycler, an Applied Biosystems® ViiA™ 7 Real-Time PCR System, an Applied Biosystems® 7500 Fast Real-Time PCR System, a 7900HT Fast Real-Time PCR System, a StepOne® Real-Time PCR System, a StepOnePlus® Real-Time PCR System, a QuantStudio™ 3 or 5 Real-time PCR System, a QuantStudio™ 6K, 7K or 12K Flex Real-Time PCR System, a QuantStudio™ Dx Real-Time PCR System and the like (all from Thermo Fisher Scientific). Other examples of spectrophotometric thermal cyclers for use in the methods include, but are not limited to, Bio-Rad iCycler iQ™, Cepheid SmartCycler® II, Corbett Research Rotor-Gene 3000, Idaho Technologies R.A.P.I.D.™, MJ Research Chromo 4™, Roche Applied Science LightCycler®, Roche Applied Science LightCycler®2.0, Stratagene Mx3000P™, and Stratagene Mx4000™. It will be recognized that a variety of instruments are commercially available and suitable for use with the methods as disclosed herein.

In some embodiments, a reverse transcription—polymerase chain reaction (RT-PCR) is performed in which both reverse transcription of a target RNA sequence and amplification of the resultant cDNA occurs in the same reaction mixture. In some embodiments, the RT-PCR is performed as a two-step or multi-step process. In other embodiments, the RT PCR is performed in a single step (e.g., 1-step RT-PCR). In some embodiments, the RT-PCR reaction mixture further includes a detectably labeled, target-specific probe such that detection of the amplified cDNA also occurs in the same reaction mixture.

In certain embodiments, an amplification reaction includes a plurality or multiplicity of single-plex reactions performed in parallel under the same assay conditions and/or at substantially the same time. In some embodiments, performing the amplification reactions in parallel forms different amplification products. In certain embodiments, performing the amplification reactions in parallel can form between 10 and 10,000 different amplification products. In some embodiments, performing the amplification reactions in parallel can form between 10 and 1000 different amplification products. In certain embodiments, performing the amplification reactions in parallel can form between 10 and 100 different amplification products or between 10 and 50 different amplification products.

In certain embodiments, an amplification reaction includes multiplex amplification, in which a multiplicity of different target nucleic acids and/or a multiplicity of different amplification product species are simultaneously amplified using a multiplicity of different primer sets. In certain embodiments, a multiplex amplification reaction and a single-plex amplification reaction, including a multiplicity of single-plex or lower-plexy reactions (for example, but not limited to, a two-plex, a three-plex, a four-plex, a five-plex or a six-plex reaction) are performed in parallel.

As described herein, exemplary methods for polymerizing and/or amplifying nucleic acids include, for example, polymerase-mediated extension reactions. For instance, the polymerase-mediated extension reaction can be the polymerase chain reaction (PCR or qPCR). In other embodiments, the nucleic acid amplification reaction is a single-plex or a multiplex PCR or qPCR reaction. For instance, exemplary methods for polymerizing and/or amplifying and detecting nucleic acids suitable for use as described herein are commercially available as TaqMan® assays (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,urine specimen 102; 6,127,155; 6,171,785; 6,214,979; 6,258,569; 6,814,934; 6,821,727; 7,141,377; and/or 7,445,900, all of which are hereby incorporated herein by reference in their entirety). TaqMan® assays are typically carried out by performing nucleic acid amplification on a target polynucleotide using a nucleic acid polymerase having 5′-to-3′ nuclease activity, at least one primer capable of hybridizing to the target polynucleotide, and an oligonucleotide probe capable of hybridizing to the target polynucleotide 3′ relative to the primer. The oligonucleotide probe typically includes a detectable label (e.g., a fluorescent reporter molecule) and a quencher molecule capable of quenching the fluorescence of the reporter molecule. Typically, though not required, the detectable label and quencher molecule are part of a single probe. As amplification proceeds, the polymerase digests the probe to separate the detectable label from the quencher molecule. The detectable label (e.g., fluorescence) is monitored during the reaction, where detection of the label corresponds to the occurrence of nucleic acid amplification (e.g., the higher the signal the greater the amount of amplification). Variations of TaqMan® assays (e.g., LNA™ spiked TaqMan® assay) are known in the art and would be suitable for use in the methods described herein.

In addition to 5′-nuclease probes, such as the probes used in TaqMan® assays, various probes are known in the art and suitable for use in detecting amplified nucleic acids in the provided methods. Exemplary probes include, but are not limited to, various stem-loop molecular beacons (e.g., U.S. Pat. Nos. 6,103,476 and 5,925,517 and Tyagi and Kramer, Nature Biotechnology 14:303-308 (1996)), stemless or linear beacons (e.g., PCT Pub. No. WO 99/21881; U.S. Pat. No. 6,485,901), PNA Molecular Beacons™ (e.g., U.S. Pat. Nos. 6,355,421 and 6,593,091), linear PNA beacons (e.g., Kubista et al., SPIE 4264:53-58 (2001)), non-FRET probes (e.g., U.S. Pat. No. 6,150,097), Sunrise®/Amplifluor® probes (U.S. Pat. No. 6,548,250), stem-loop and duplex Scorpions™ probes (Solinas et al., Nucleic Acids Research 29:E96 (2001) and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes (Svanvik, et al. Anal Biochem 281:26-35 (2000)), self-assembled nanoparticle probes, ferrocene-modified probes described, for example, in U.S. Pat. No. 6,485,901; Mhlanga et al., Methods 25:463-471 (2001); Whitcombe et al., Nature Biotechnology. 17:804-807 (1999); Isacsson et al., Molecular Cell Probes. 14:321-328 (2000); Wolffs et al., Biotechniques 766:769-771 (2001); Tsourkas et al., Nucleic Acids Research. 30:4208-4215 (2002); Riccelli et al., Nucleic Acids Research 30:4088-4093 (2002); Zhang et al., Acta Biochimica et Biophysica Sinica (Shanghai). 34:329-332 (2002); Maxwell et al., J. Am. Chem. Soc. 124:9606-9612 (2002); Broude et al., Trends Biotechnol. 20:249-56 (2002); Huang et al., Chem Res. Toxicol. 15:118-126 (2002); and Yu et al., J. Am. Chem. Soc. 14:11155-11161 (2001); QuantiProbes® (Qiagen), HyBeacons® (French, et al. Mol. Cell. Probes 15:363-374 (2001)), displacement probes (Li, et al. Nucl. Acids Res. 30:e5 (2002)), HybProbes (Cardullo, et al. Proc. Natl. Acad. Sci. USA 85:8790-8794 (1988)), MGB Alert (www.nanogen.com), Q-PNA (Fiandaca, et al. Genome Res. 11:609-611 (2001)), Plexor™ (Promega), LUX™ primers (Nazarenko, et al. Nucleic Acids Res. 30:e37 (2002)), DzyNA primers (Todd, et al. Clin. Chem. 46:625-630 (2000)). Detectably-labeled probes may also include non-detectable quencher moieties that quench the fluorescence of the detectable label, including, for example, black hole quenchers (Biosearch), Iowa B1ack™ quenchers (IDT), QSY quencher (Molecular Probes™; Thermo Fisher Scientific), and Dabsyl and Dabcyl sulfonate/carboxylate Quenchers (Epoch). Detectably-labeled probes may also include two probes, wherein for example a fluorophore is on one probe, and a quencher is on the other, wherein hybridization of the two probes together on a target quenches the signal, or wherein hybridization on a target alters the signal signature via a change in fluorescence. Exemplary systems may also include FRET, salicylate/DTPA ligand systems (Oser et al. Angew. Chem. Int. Engl. 29(10):1167 (1990)), displacement hybridization, homologous probes, and/or assays described in European Pat. No. EP 070685 and/or U.S. Pat. No. 6,238,927. Detectable labels can also include sulfonate derivatives of fluorescein dyes with S03 instead of the carboxylate group, phosphoramidite forms of fluorescein, phosphoramidite forms of Cy5 (available for example from Amersham). All references cited above are hereby incorporated herein by reference in their entirety.

As used herein, the term “detectable label” refers to any of a variety of signaling molecules indicative of nucleic acid synthesis and/or amplification. The reaction mixture may include a detectable label such as SYBR® Green and/or other DNA-binding dyes. Such detectable labels may include or may be, for example, nucleic acid intercalating agents or non-intercalating agents. As used herein, an intercalating agent is an agent or moiety capable of non-covalent insertion between stacked base pairs of a double-stranded nucleic acid molecule. A non-intercalating agent is one that does not insert into the double-stranded nucleic acid molecule. The nucleic acid binding agent may produce a detectable signal directly or indirectly. The signal may be detectable directly using, for example, fluorescence and/or absorbance, or indirectly using, for example, any moiety or ligand that is detectably affected by proximity to a double-stranded nucleic acid molecule. As used herein, an intercalating agent is an agent or moiety capable of non-covalent insertion between stacked base pairs of a double-stranded nucleic acid molecule. A non-intercalating agent acid is suitable such as a substituted label moiety or binding ligand attached to the nucleic acid binding agent. It is typically necessary for the nucleic acid binding agent to produce a detectable signal when bound to a double-stranded nucleic acid such that it is distinguishable from the signal produced when that same agent is in solution or bound to a single-stranded nucleic acid. For example, intercalating agents such as ethidium bromide fluoresce more intensely when intercalated into double-stranded DNA than when bound to single-stranded DNA, RNA, or in solution (e.g., U.S. Pat. Nos. 5,994,056; 6,171,785; and/or 6,814,934). Similarly, actinomycin D fluoresces in the red portion of the UV/VIS spectrum when bound to single-stranded nucleic acids, and fluoresces in the green portion of the UV/VIS spectrum when bound to double-stranded nucleic acids. And in yet another example, the photoreactive psoralen 4-aminomethyl-4-5′,8-trimethylpsoralen (AMT) has been reported to exhibit decreased absorption at long wavelengths and fluorescence upon intercalation into double-stranded DNA (Johnson et al. Photochem. & Photobiol., 33:785-791 (1981). For example, U.S. Pat. No. 4,257,774 describes the direct binding of fluorescent intercalators to DNA (e.g., ethidium salts, daunomycin, mepacrine and acridine orange, 4′,6-diamidino-α-phenylindole). Non-intercalating agents (e.g., minor groove binder moieties (MGBs), such as Hoechst 33258, distamycin, netropsin, may also be suitable for use with the compositions, methods and kits as described herein. For example, Hoechst 33258 (Searle, et al. Nucl. Acids Res. 18(13):3753-3762 (1990)) exhibits altered fluorescence with an increasing amount of a target nucleic acid.

As described herein, one or more detectable labels and/or quenching agents may be attached to one or more primers and/or probes (e.g., detectable label). The detectable label may emit a signal when free or when bound to one of the target nucleic acids. The detectable label may also emit a signal when in proximity to another detectable label. Detectable labels may also be used with quencher molecules such that the signal is only detectable when not in sufficiently close proximity to the quencher molecule. For instance, in some embodiments, the assay system may cause the detectable label to be liberated from the quenching molecule. Any of several detectable labels may be used to label the primers and probes used in the methods described herein. As described herein, in some embodiments the detectable label may be attached to a probe, which may be incorporated into a primer, or may otherwise bind to amplified target nucleic acid (e.g., a detectable nucleic acid binding agent such as an intercalating or non-intercalating dye). When using more than one detectable label, each should differ in their spectral properties such that the labels may be distinguished from each other, or such that together the detectable labels emit a signal that is not emitted by either detectable label alone. Exemplary detectable labels include, for instance, a fluorescent dye or fluorphore (e.g., a chemical group that can be excited by light to emit fluorescence or phosphorescence), “acceptor dyes” capable of quenching a fluorescent signal from a fluorescent donor dye, and the like. Suitable detectable labels may include, for example, fluorosceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); FITC; 6-carboxy-1,4-dichloro-2′,7′-dichlorofluorescein (TET); 6-carboxy-1,4-dichloro-2′,4′,5′,7′-tetrachlorofluorescein (HEX); 6-carboxy-4′,5′-dichloro-2′,7′-dimethoxyfluorescein (JOE); Alexa fluor® fluorophores (e.g., 350, 405, 430, 488, 500, 514, 532, 546, 555, 568, 594, 610, 633, 635, 647, 660, 680, 700, 750); BODIPY™ fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), coumarins (e.g., 7-amino-4-methylcoumarin, AMC, AMCA, AMCA-S, AMCA-X, ABQ, CPM methylcoumarin, coumarin phalloidin, hydroxycoumarin, CMFDA, methoxycoumarin), calcein, calcein AM, calcein blue, calcium dyes (e.g., calcium crimson, calcium green, calcium orange, calcofluor white), Cascade Blue, Cascade Yellow; CyTM dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/tetramethylrhodamine, IAEDANS/fluorescein, EDANS/dabcyl, fluorescein/fluorescein, BODIPY® FL/BODIPY® FL, Fluorescein/QSY7 and QSY9), LysoTracker® and LysoSensor™ (e.g., LysoTracker® Blue DND-22, LysoTracker® Blue-White DPX, LysoTracker® Yellow HCK-123, LysoTracker® Green DND-26, LysoTracker® Red DND-99, LysoSensor™ Blue DND-167, LysoSensor™ Green DND-189, LysoSensor™ Green DND-153, LysoSensor™ Yellow/Blue DND-160, LysoSensor™ Yellow/Blue 10,000 MW dextran), Oregon Green (e.g., 488, 488-X, 500, 514); rhodamines (e.g., real time PCR detection system 110, 123, B, B 200, BB, BG, B extra, 5-carb oxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethylrhodamine), Tetramethylrhodamine (TRITC), WT), Texas Red, Texas Red-X, VIC and other labels described in, e.g., U.S. Pat. Pub. No. 2009/0197254 (incorporated herein by reference in its entirety), among others as would be known to those of skill in the art. Other detectable labels may also be used (see, e.g., U.S. Pat. Pub. No. 2009/0197254 (incorporated herein by reference in its entirety)), as would be known to those of skill in the art. Any of these systems and detectable labels, as well as many others, may be used to detect amplified target nucleic acids.

Other DNA binding dyes are available to one of skill in the art and may be used alone or in combination with other agents and/or components of an assay system. Exemplary DNA binding dyes may include, for example, acridines (e.g., acridine orange, acriflavine), actinomycin D (Jain, et al. J. Mol. Biol. 68:21 (1972)), anthramycin, BOBO™-1, BOBO™-3, BO-PRO™-1, cbromomycin, DAPI (Kapuseinski, et al. Nucl. Acids Res. 6(112): 3519 (1979)), daunomycin, distamycin (e.g., distamycin D), dyes described in U.S. Pat. No. 7,387,887, ellipticine, ethidium salts (e.g., ethidium bromide), fluorcoumanin, fluorescent intercalators as described in U.S. Pat. No. 4,257,774, GelStar® (Lonza), Hoechst 33258 (Searle and Embrey, Nucl. Acids Res. 18:3753-3762 (1990)), Hoechst 33342, homidium, JO-PRO™-1, LIZ dyes, LO-PRO™-1, mepacrine, mithramycin, NED dyes, netropsin, 4′,6-diamidino-α-phenylindole, proflavine, POPO™-1, POPO™-3, PO-PRO™-1, propidium iodide, ruthenium polypyridyls, S5, SYBR® Gold, SYBR® Green I (U.S. Pat. Nos. 5,436,134 and 5,658,751), SYBR® Green II, SYTOX® blue, SYTOX® green, SYTO® 43, SYTO® 44, SYTO® 45, SYTOX® Blue, TO-PRO®-1, SYTO® 11, SYTO® 13, SYTO® 15, SYTO® 16, SYTO® 20, SYTO® 23, thiazole orange (Sigma-Aldrich Chemical Co.), TOTO™-3, YO-PRO®-1, and YOYO®-3 (Molecular Probes; Thermo Fisher Scientific), among others. SYBR® Green I (e.g., U.S. Pat. Nos. 5,436,134; 5,658,751; and/or 6,569,927), for example, has been used to monitor a PCR reactions. Other DNA binding dyes may also be suitable as would be understood by one of skill in the art.

In some aspects, detection of the detectable label or signal may be done using any reagents or instruments that detect a change in fluorescence from a fluorophore. For example, detection may be performed using any spectrophotometric thermal cycler. Examples of spectrophotometric thermal cyclers include, but are not limited to, Applied Biosystems (AB) PRISM® 7000, AB 7300 real-time PCR system, AB 7500 real-time PCR system, AB PRISM™ 7900HT, Bio-Rad ICycler IQ™, Cepheid SmartCycler® II, Corbett Research Rotor-Gene 3000, Idaho Technologies R.A.P.I.D.™, MJ Research Chromo 4™, Roche Applied Science LightCycler®, Roche Applied Science LightCycler®2.0, Stratagene Mx3000P™, and Stratagene Mx4000™. It should be noted that new instruments are being developed at a rapid rate and any like instruments may be used for the methods.

The nucleic acid polymerases that may be employed in the disclosed nucleic acid amplification reactions may be any that function to carry out the desired reaction including, for example, a prokaryotic, fungal, viral, bacteriophage, plant, and/or eukaryotic nucleic acid polymerase. As used herein, the term “DNA polymerase” refers to an enzyme or polypeptide that synthesizes a DNA strand de novo using a nucleic acid strand as a template. In general, DNA polymerases use an existing DNA or RNA as a template for DNA synthesis and catalyze the polymerization of deoxyribonucleotides alongside the template strand, which it reads for incorporation of the appropriate nucleotide. The newly synthesized DNA strand is complementary to the template strand. DNA polymerase can add free nucleotides only to the 3′-hydroxyl end of the newly forming strand. It synthesizes oligonucleotides via transfer of a nucleoside monophosphate from a deoxyribonucleoside triphosphate (dNTP) to the 3′-hydroxyl group of a growing oligonucleotide chain. This results in elongation of the new strand in a 5′-to-3′ direction. Since DNA polymerase can only add a nucleotide onto a pre-existing 3′-OH group, to begin a DNA synthesis reaction, the DNA polymerase needs a primer to which it can add the first nucleotide. Suitable primers may include oligonucleotides of RNA or DNA, or chimeras thereof (e.g., RNA/DNA chimerical primers). The DNA polymerases may be a naturally occurring DNA polymerases or a variant of natural enzyme having the above-mentioned activity. For example, it may include a DNA polymerase having a strand displacement activity, a DNA polymerase lacking 5′-to-3′ exonuclease activity, a DNA polymerase having a reverse transcriptase activity, or a DNA polymerase having an endonuclease activity.

Polymerases used in accordance with the present teachings may be any enzyme that can synthesize a nucleic acid molecule from a nucleic acid template, typically in the 5′ to 3′ direction. Suitable nucleic acid polymerases may also include holoenzymes, functional portions of the holoenzymes, a chimeric or fusion polymerase or polypeptide having polymerase activity, or any modified polymerase that can effectuate the synthesis of a nucleic acid molecule. Within this disclosure, a DNA polymerase may also include a polymerase, terminal transferase, reverse transcriptase, telomerase, polynucleotide phosphorylase and/or any polypeptide having polymerase activity.

The nucleic acid polymerases used in the methods disclosed herein may be mesophilic or thermophilic. Exemplary mesophilic DNA polymerases include T7 DNA polymerase, T5 DNA polymerase, Klenow fragment DNA polymerase, DNA polymerase III and the like. Non-limiting examples of polymerases may include, for example, T7 DNA polymerase, eukaryotic mitochondrial DNA Polymerase y, prokaryotic DNA polymerase I, II, III, IV, and/or V; eukaryotic polymerase α, β, γ, δ, ε, η, ζ, ι, and/or κ; E. coli DNA polymerase I; E. coli DNA polymerase III alpha and/or epsilon subunits; E. coli polymerase IV, E. coli polymerase V; T. aquaticus DNA polymerase I; B. stearothermophilus DNA polymerase I; Euryarchaeota polymerases; terminal deoxynucleotidyl transferase (TdT); S. cerevisiae polymerase 4; translesion synthesis polymerases; reverse transcriptase; and/or telomerase. Non-limiting examples of suitable thermostable DNA polymerases that may be used include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Bacillus sterothermophilus (Bst) DNA polymerase, Bacillus caldophilus (Bca) DNA polymerase, Sulfobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), and mutants, and variants and derivatives thereof (U.S. Pat. Nos. 5,436,149; 4,889,818; 4,965,188; 5,079,352; 5,614,365; 5,374,553; 5,270,179; 5,047,342; 5,512,462; WO 92/06188; WO 92/06200; WO 96/10640; Barnes, Gene 112:29-35 (1992); Lawyer, et al., PCR Meth. Appl. 2:275-287 (1993); Flaman, et al., Nucl. Acids Res. 22(15):3259-3260 (1994)). RNA polymerases such as T3, T5 and SP6 and mutants, variants and derivatives thereof may also be used in accordance with the present teachings. Generally, any type I DNA polymerase may be used in accordance with the invention although other DNA polymerases may be used including, but not limited to, type III or family A, B, C etc. DNA polymerases. In addition, any genetically engineered DNA polymerases, any having reduced or insignificant 3′-to-5′ exonuclease activity (e.g., SuperScript™ DNA polymerase), and/or genetically engineered DNA polymerases (e.g., those having the active site mutation F667Y or the equivalent of F667Y (e.g., in Tth), AmpliTaq™FS, ThermoSequenase™), AmpliTaq™ Gold, Platinum™ Taq DNA Polymerase, Therminator I, Therminator II, Therminator III, Therminator Gamma (New England Biolabs, Beverly, Mass.), and/or any derivatives and fragments thereof, may be used in accordance with the present teachings. Examples of DNA polymerases substantially lacking in 3′ exonuclease activity include, but are not limited to, Taq, Tne(exo-), Tma(exo-), Pfu (exo-), Pwo(exo-) and Tth DNA polymerases, and mutants, variants and derivatives thereof. Other nucleic acid polymerases may also be suitable as would be understood by one of skill in the art.

Enzymes for use in the methods, compositions and kits provided herein may also include any enzyme or polypeptide having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, et al., Science 239:487-491 (1988); U.S. Pat. Nos. 4,889,818 and 4,965,188), Tne DNA polymerase (WO 96/10640), Tma DNA polymerase (U.S. Pat. No. 5,374,553) and mutants, fragments, variants or derivatives thereof (see, e.g., U.S. Pat. Nos. 5,948,614 and 6,015,668, which are incorporated by reference herein in their entireties). As will be understood by one of ordinary skill in the art, modified reverse transcriptases and DNA polymerase having reverse transcriptase activity may be obtained by recombinant or genetic engineering techniques that are well-known in the art. Mutant reverse transcriptases or polymerases may, for example, be obtained by mutating the gene or genes encoding the reverse transcriptase or polymerase of interest by site-directed or random mutagenesis. Such mutations may include point mutations, deletion mutations and insertional mutations. In some embodiments, one or more point mutations (e.g., substitution of one or more amino acids with one or more different amino acids) are used to construct mutant reverse transcriptases or polymerases for use in the invention. Fragments of reverse transcriptases or polymerases may also be obtained by deletion mutation by recombinant techniques that are well-known in the art, or by enzymatic digestion of the reverse transcriptase(s) or polymerase(s) of interest using any of a number of well-known proteolytic enzymes.

Exemplary polypeptides having reverse transcriptase activity for use in the methods provided herein include Moloney Murine Leukemia Virus (M-MLV) reverse transcriptase, Rous Sarcoma Virus (RSV) reverse transcriptase, Avian Myeloblastosis Virus (AMV) reverse transcriptase, Rous Associated Virus (RAV) reverse transcriptase, Myeloblastosis Associated Virus (MAV) reverse transcriptase and Human Immunodeficiency Virus (HIV) reverse transcriptase, and others described in WO 98/47921 and derivatives, variants, fragments or mutants thereof, and combinations thereof. In a further embodiment, the reverse transcriptases are reduced or substantially reduced in RNase H activity, and may be selected from the group consisting of M-MLV H- reverse transcriptase, RSV H- reverse transcriptase, AMV H- reverse transcriptase, RAV H- reverse transcriptase, MAV H- reverse transcriptase and HIV H- reverse transcriptase, and derivatives, variants, fragments or mutants thereof, and combinations thereof. Reverse transcriptases of particular interest include AMV RT and M-MLV RT, and optionally AMV RT and M-MLV RT having reduced or substantially reduced RNase H activity (e.g., AMV RT alpha H-/BH+ and M-MLV RT H−). Reverse transcriptases for use in the invention include SuperScript™, SuperScript™ II, ThermoScript™ and ThermoScript™ II available from Invitrogen™ (Thermo Fisher Scientific). See generally, WO 98/47921, U.S. Pat. Nos. 5,244,797 and 5,668,005, the entire contents of each of which are herein incorporated by reference.

Polypeptides having reverse transcriptase activity for use in the methods provided herein may be obtained commercially, for example, from Invitrogen™ (Thermo Fisher Scientific), Pharmacia (Piscataway, N.J.), Sigma (Saint Louis, Mo.) or Boehringer Mannheim Biochemicals (Indianapolis, Ind.). Alternatively, polypeptides having reverse transcriptase activity may be isolated from their natural viral or bacterial sources according to standard procedures for isolating and purifying natural proteins that are well-known to one of ordinary skill in the art (see, e.g., Houts, et al., J. Virol. 29:517 (1979)). In addition, the polypeptides having reverse transcriptase activity may be prepared by recombinant DNA techniques that are familiar to one of ordinary skill in the art (see, e.g., Kotewicz, et al., Nucl. Acids Res. 16:265 (1988); Soltis and Skalka, Proc. Natl. Acad. Sci. USA 85:3372-3376 (1988)).

DNA polymerases for use in the compositions, methods and kits as disclosed herein may be obtained commercially, for example, from Invitrogen™ (Thermo Fisher Scientific), Pharmacia (Piscataway, N.J.), Sigma (St. Louis, Mo.), Boehringer Mannheim, and New England Biolabs (Beverly, Mass.).

Kits for performing the methods described herein are also provided. Kits including an amplification control nucleic acid for performing the methods described herein are also provided. As used herein, the term “kit” refers to a packaged set of related components, typically one or more compounds or compositions. In some embodiments, the kit may include at least one amplification control nucleic acid composition and may further include a pair of oligonucleotides or primers for polymerizing and/or amplifying at least one target sequence from the control nucleic acid, a nucleic acid polymerase, and/or a corresponding one or more probes labeled with a detectable label for detection of the control nucleic acid. The kit may include at least one amplification control nucleic acid composition including at least one amplification control nucleic acid molecule, such as in the form of a plasmid, or superplasmid as described herein, and may further include a pair of oligonucleotides for polymerizing and/or amplifying at least one target sequence from the control nucleic acid molecule, a nucleic acid polymerase, and/or corresponding one or more probes labeled with a detectable label for detection of the control nucleic acid. The kits may also include samples including other pre-defined target nucleic acids to be used in control reactions. The kits may also include a pair of oligonucleotides or primers for polymerizing and/or amplifying at least one target nucleic acid from a biological sample. The kits may also optionally include stock solutions, buffers, enzymes, detergents, amplification stabilizing components, RNase inhibitor components, detectable labels or other reagents used for amplification and/or detection, tubes, membranes, and the like that may be used to complete the amplification reaction. In some embodiments, multiple primer sets are included. In one embodiment, the kit may include one or more of, for example, a buffer (e.g., Tris), one or more salts (e.g., KC1), glycerol, dNTPs (dA, dT, dG, dC, dU), recombinant BSA (bovine serum albumin), a dye (e.g., ROX passive reference dye), one or more detergents (e.g., Triton X-100, Nonidet P-40, Tween 20, Brij-58), polyethylene glycol (PEG), polyvinyl pyrrolidone (PVP), gelatin (e.g., fish or bovine source) and/or an antifoam agent provided in one or more containers. Other embodiments of particular systems and kits are also contemplated which would be understood by one of skill in the art.

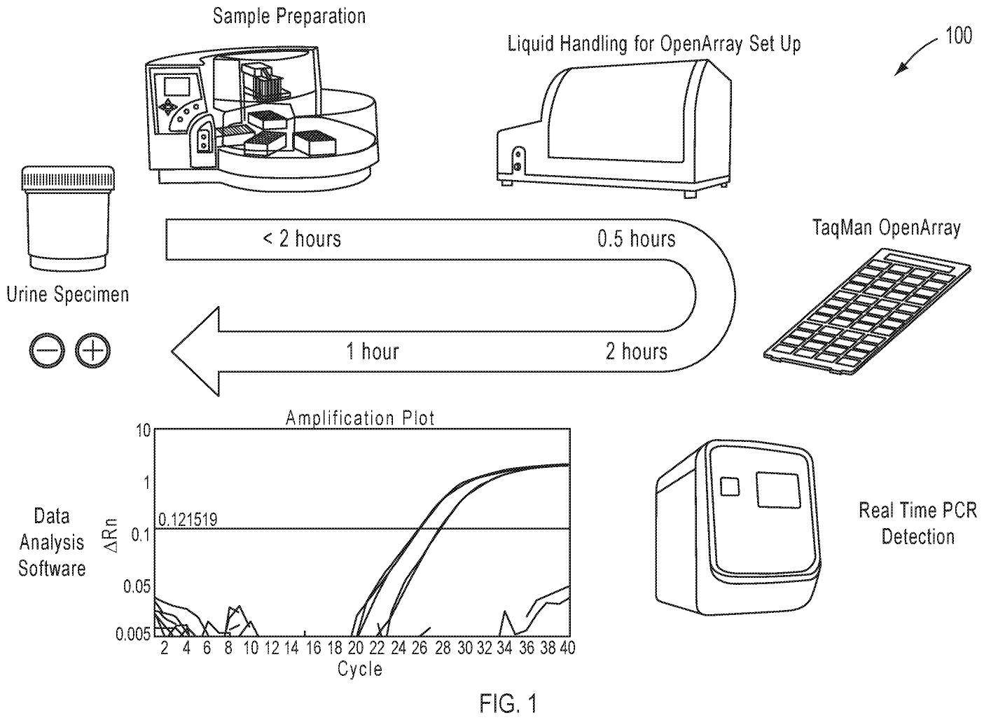

In some embodiments, referencing FIG. 1, a workflow 100 for amplifying nucleic acid sequences involves collecting a urine specimen and performing sample preparation on the sample using any system or method readily available and/or known to those of skill in the art. In some embodiments, the sample prep system extracts a nucleic acid sample from the microbial cells in the urine for subsequent utilization in an open array microfluidic plate. The open array microfluidic plate includes plurality of through-holes with each through-hole including a hydrophobic exterior and a hydrophilic interior. The interior of the through-hole is spotted with an assay selected for the amplification reaction. When the prepared sample is loaded on to the open array microfluidic plate, the fluidic properties of the plate retains an equal volume of the sample in each through-hole. In some embodiments, once the open array microfluidic plate is loaded it is transferred to a real time PCR or quantitative PCR detection system to undergo an amplification reaction. During the amplification reaction, in some embodiments, the real time/quantitative PCR detection system detects formation of the amplicons through the detection of a florescent dye. The detection of the florescent dye indicates the presences of a microorganisms corresponding to the assay utilized within the particular through-hole.

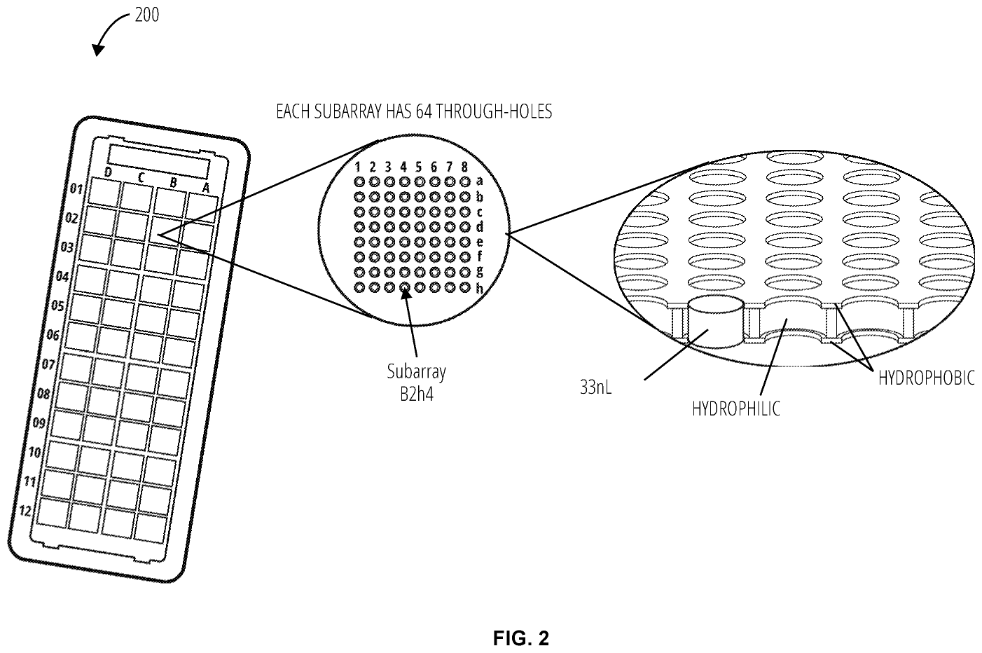

In some embodiments, referencing FIG. 2 a reaction vessel 200 such as a microscope slide-sized plate which includes a micro-subarray each including through-hole is utilized. In some embodiments, each plate comprises 3,072 through-holes or reaction sites. In some embodiments, each plate contains 48 subarrays with 64 through-holes. In some embodiments, each through-hole is 300 μm in diameter and 300 μm in depth. In some embodiments, each of the through-holes includes a hydrophobic exterior and a hydrophilic interior. In some embodiments, the hydrophilic interior is spotted with an assay, such as those listed in Table 1. In some embodiments, reaction mixtures are retained in the through-holes via surface tension.

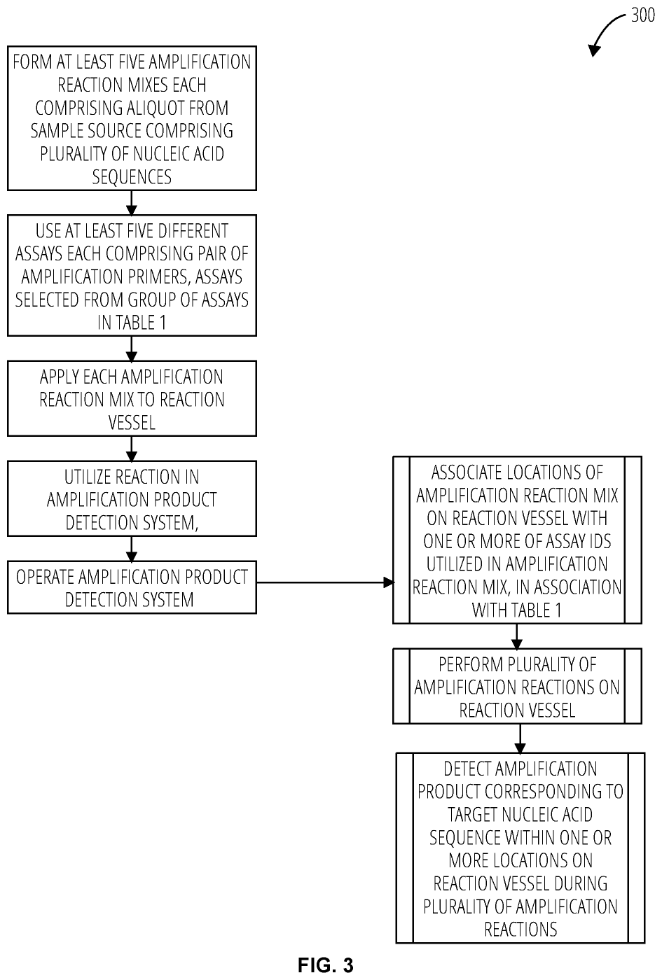

In some embodiments, referencing FIG. 3, a method 300 for amplifying nucleic acid sequences in a nucleic acid sample involves forming at least five amplification reaction mixes each including an aliquot from a sample source including nucleic acid sequences. In some embodiments, the method uses at least five different assays each including a pair of amplification primers, the assays selected from the group of assays in Table 1. In some embodiments, the method applies each amplification reaction mix to a reaction vessel. In some embodiments, the method utilizes the reaction in an amplification product detection system. In some embodiments, the method operates the amplification product detection system. In some embodiments, the amplification product detection system associate locations of the amplification reaction mix on the reaction vessel with one or more of the assay IDs utilized in the amplification reaction mix, in an association table. In some embodiments, the amplification product detection system performs amplification reactions on the reaction vessel. In some embodiments, the amplification product detection system detects an amplification product corresponding to a target nucleic acid sequence within one or more locations on the reaction vessel during the amplification reactions.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following examples, which should not be construed as limiting the scope of the teachings in any way.

### EXAMPLES

A panel of TaqMan™ Assays was designed to detect and/or profile urinary tract microbiota (UTM) by targeting signature genes associated with various UTM. The panel of such assays was designed to discriminate between 17 different species of microorganisms which include pathogenic microbes associated with the bladder, urinary tract and the urogenital area. The panel includes assays to detect the microorganisms (bacteria, and/or fungi) including those listed in Table 1. Of the 17 species listed in Table 1, most of them are bacterial covering a wide range of bacteria (13 gram negative and 3 gram positive). The panel also covers one fungal target. All of microorganisms listed in Table 1 are closely associated with urinary tract health.

For the panel, florescent labeled assays were spotted onto high throughput OpenArray™ plates, as illustrated in FIG. 2. A plasmid (e.g., superplasmid) containing amplicon sequences specific to each assay listed in Table 1 was also designed and prepared as described herein. The superplasmid DNA was quantified by digital PCR using QuantStudio™ 3D Digital PCR System. The superplasmid containing synthetic sequences for the amplicons of all the UTM assays listed in Table 1 was constructed and was used as a positive control. Genomic DNA (gDNA) controls of inclusivity and exclusivity panels were purchased from ATCC. The panel assays were evaluated with synthetic superplasmid and/or ATCC genomic DNA samples using TaqMan® OpenArray® Real-Time PCR Master Mix (Thermo Fisher) on QuantStudio™ 12Flex Real Time PCR System™. The workflow and system used for evaluation of the assay panels is described in further detail herein and also illustrated in FIGS. 1 and 3.

For the amplification control nucleic acid molecule, a DNA sequence was designed and the corresponding DNA molecule synthesized to include all the target amplicons and a portion of their flanking regions for the microbe-specific assays in the panel described above, as well as several control templates, including a 100-200 nucleotide xeno sequence and a sequence fragment from the human RNase P gene sequence. A unique restriction site for downstream linearization was also engineered into the DNA sequence including the target amplicons and a portion of each of their corresponding 5′- and 3′-flanking sequences. The synthesized DNA molecule was cloned into a bacterial plasmid vector to create a multi-target plasmid (i.e., superplasmid). In these examples, the superplasmid was designed to include target sequences for the panel of 17 assays listed in Table 1 (“UTM superplasmid”) along with other control sequences as mentioned above. After transformation of the superplasmid into E. coli and subsequent plasmid DNA extraction, the plasmid was linearized at the unique restriction site by restriction enzyme digestion and the plasmid preparation was quantified. The linearized control plasmid preparation was normalized to a final concentration of 1×10⁷ copies/microliter and was diluted serially from a concentration of 1×10⁷ copies/microliter to 1×10² copies/microliter, and used at the indicated concentrations mentioned below.

### Example 1

Amplification of linearized control plasmid preparations was tested using TaqMan™ OpenArray™ plates (Applied Biosystems) pre-spotted with a panel of 17 different TaqMan™ assays described above plus two control assays (Xeno and RNase P). Each assay included a pair of amplification primers and an oligonucleotide TaqMan™ probe with a detectable label. The TaqMan™ amplification primers and probe were designed to be target specific for the corresponding genes listed for each assay as shown in Table 1. The amplification reactions were run and analyzed on a QuantStudio™ 12K Flex Real-Time PCR System according to the manufacturer's instructions (Applied Biosystems).

Prior to amplification, the amplification control plasmid preparation was serially diluted across 5 logs, from 10⁷ copies per microliter to 10² copies per microliter. For each subarray, a PCR reaction mix was prepared by adding 2.5 microliters of diluted control plasmid preparation to 2.5 microliters TaqMan™ OpenArray Real-Time PCR Master Mix (Thermo Fisher) according to the manufacturer's instructions. Five microliters of the PCR reaction mix with the control nucleic acid samples at varying concentrations was loaded on the OpenArray™ plates using an OpenArray Accufill System and run on the QuantStudio™ 12K Flex System (Thermo Fisher) per the manufacturer's instructions. Four replicates were run for each dilution.

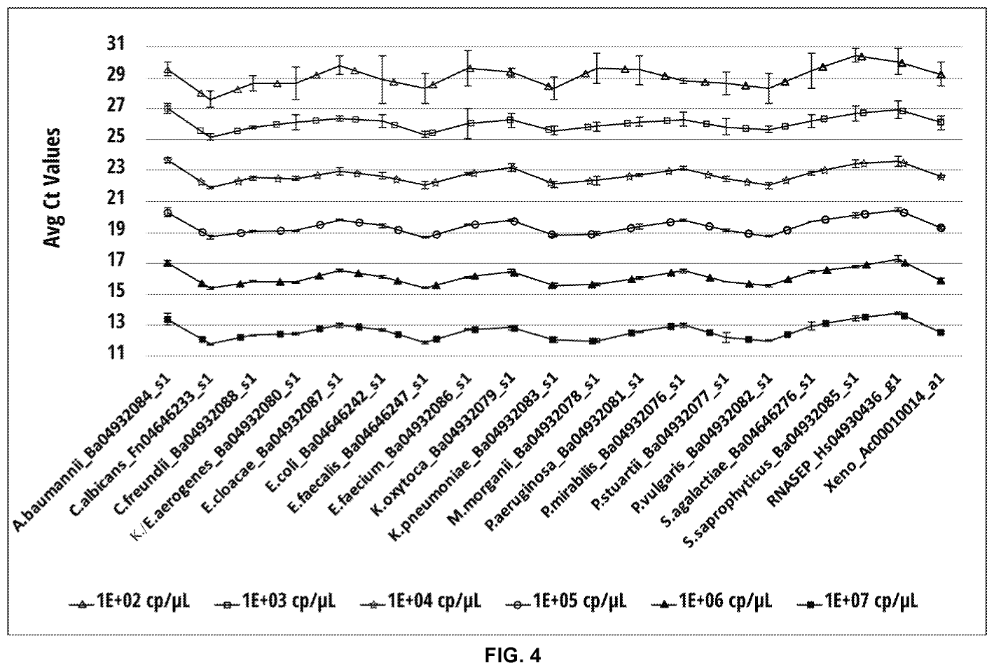

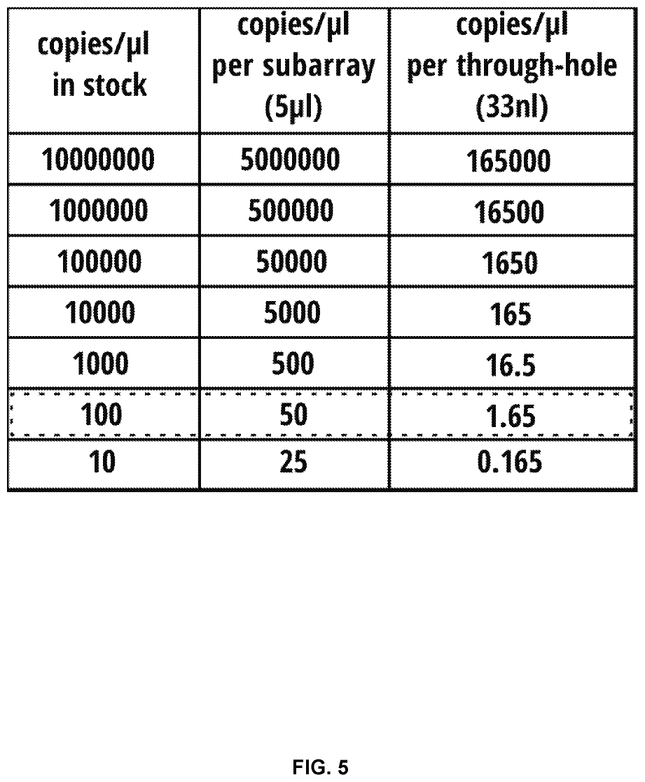

All assays tested showed a limit of detection (LOD) down to at least 100 copies/microliter. Good PCR sensitivity was achieved with the linearized control plasmid with each of the different assays tested. FIG. 4 illustrates the analytical sensitivity of the assays utilizing the superplasmid control DNA as the input sample. In FIG. 4, a serial dilution was performed with the UTM superplasmid containing the templates for all the assays from 10⁷ copies/μl stock to 10² copies/μl. FIG. 5 illustrates a conversion table showing options for presenting copies/μl in the context of stock solution, PCR reaction per sub-array (5 μl) or per through-hole (33 nl).

### Example 2

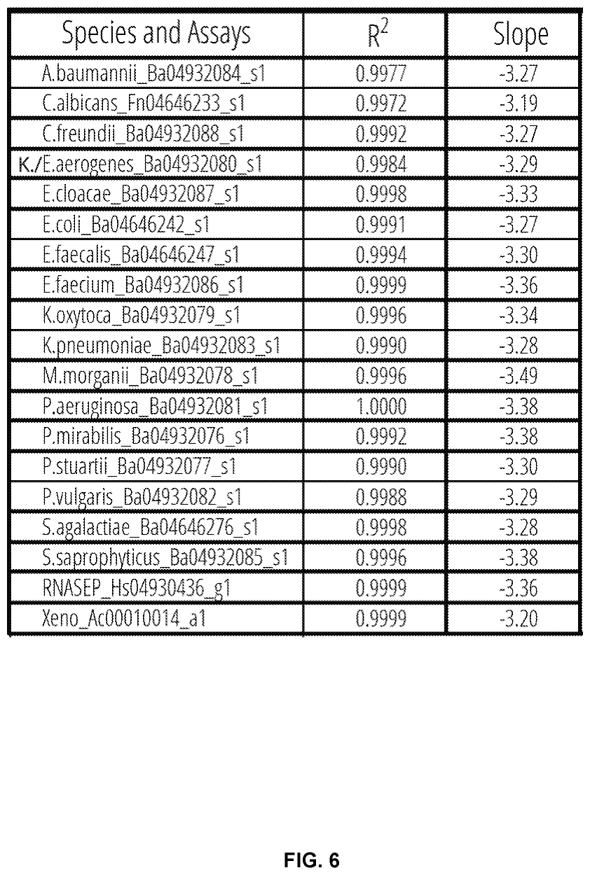

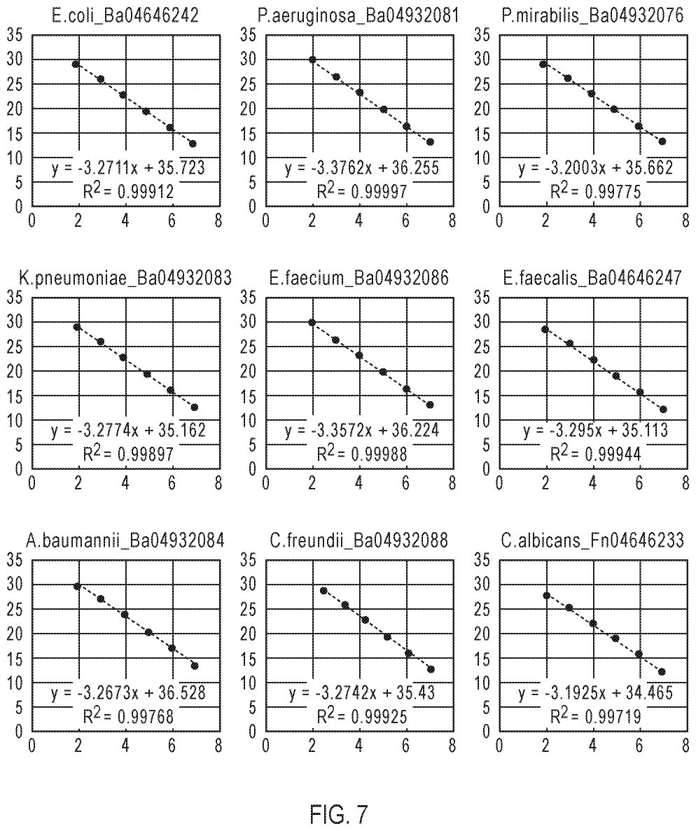

FIG. 6 and FIG. 7 illustrate experimental results from a study testing the dynamic range of the urinary tract microbiota (UTM) assays (TaqMan™ assays) on an open array as described above. A 1:10 serial dilution was performed with the UTM superplasmid from a stock solution of 10⁷ down to 10² copies/μl crossing five logs. PCR reactions were prepared by mixing 2.5 μl diluted control plasmid to 2.5 μl Master mix for each subarray containing 64 through-holes. Each subarray was spotted with 56 assays and each dilution was run in four replicates. FIG. 6 summarizes the R-square and slope of the serial dilution for each target/assay. Good PCR efficiency and reproducibility were achieved with the linearized control plasmid with each of the different assays tested on OpenArray™ plate (FIG. 6). FIG. 7 illustrates nine of the assays shown as scatter plots. For each plot, the X-axis is logio of superplasmid control template concentration (copies/μl) and the Y axis is the Ct values at each concentration. As demonstrated in FIGS. 6 and FIG. 7, limit of detection (LOD) of all assays tested was at least as low as 100 copies/microliter, crossing at least 5 logs of dilution and having an R² greater than 0.99. Together this data demonstrates good dynamic range across at least 5 logs and strong and reproducible linearity.

### Example 3

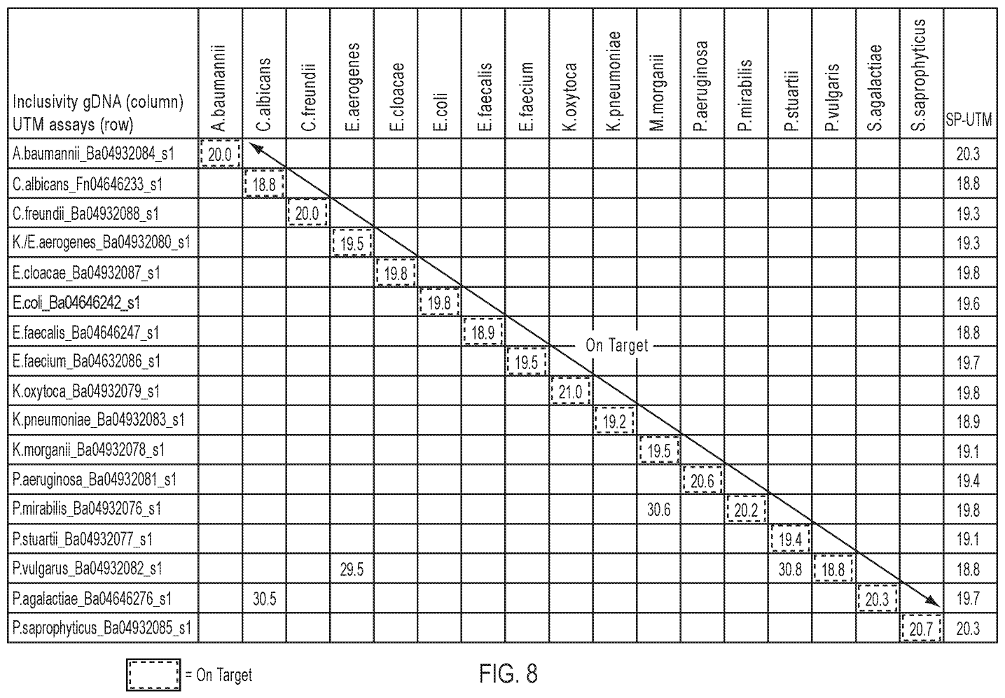

A panel of 17 different UTM TaqMan™ assays listed in Table 1 was evaluated for their accuracy and specificity using a panel of gDNA samples purchased from ATCC microbial cultures inclusive of the tested targets (FIG. 8) and a panel of gDNA samples purchased from

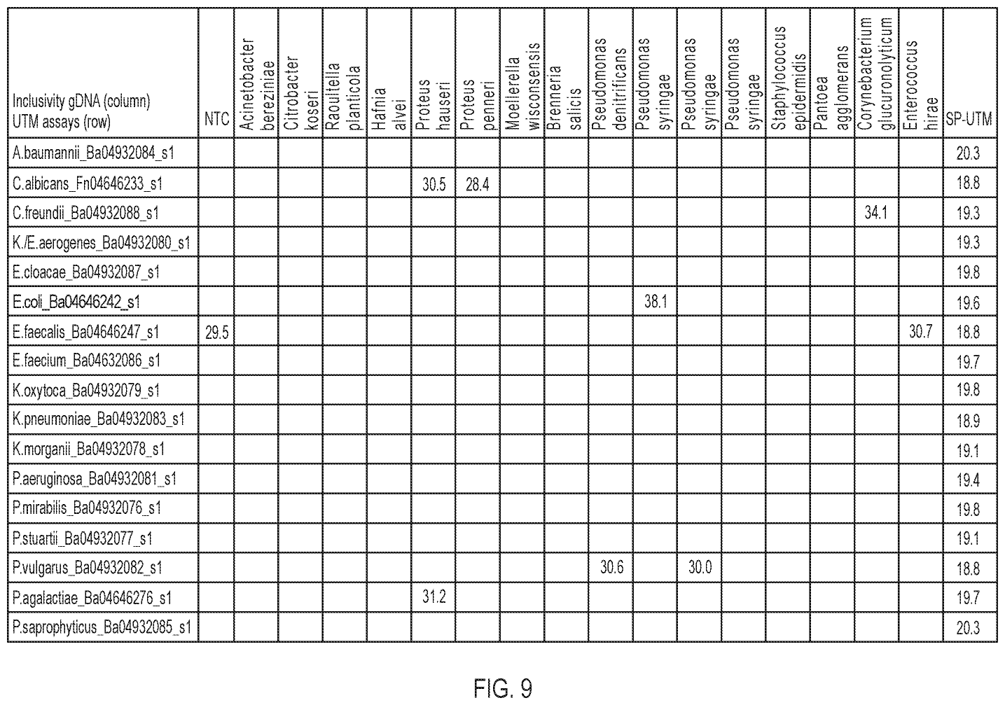

ATCC microbial cultures exclusive of the tested targets (FIG. 9). ATCC gDNA samples were quantified by dPCR using QuantStudio™ 3D Digital PCR System (Thermo Fisher). In both FIG. 8 and FIG. 9, each row represents the TaqMan Assay ID No. used for detection of the corresponding microorganism as listed in Table 1. In FIG. 8, the columns represent the sample type used for each assay, including various ATCC gDNA samples inclusive of the tested targets from microorganisms as shown and a superplasmid nucleic acid molecule/positive control sample (last column), prepared as described herein. In FIG. 9, the columns represent the sample type used for each assay, including a “NTC”-no template control/negative control sample (first column); various ATCC gDNA samples exclusive of the tested targets from various microorganism as shown; and a superplasmid nucleic acid molecule/positive control sample (last column), prepared as described herein. All the gDNA samples tested were used at a concentration of 10⁵ copies/μl based on dPCR readouts. The UTM superplasmid (“SP-UTM”), included as a positive control in both FIG. 8 and FIG. 9, was also used at a concentration of 10⁵ copies/W. A volume of 2.5μ1 of each control sample was mixed with 2.5μ1 TaqMan™ OpenArray™ Real-Time PCR Master Mix (Thermo Fisher) to make a total of 5μl PCR reaction. The PCR reactions were loaded onto each subarray of an OpenArray™ plate on which all the UTM assays are spotted as described above. The OpenArray™ plates were thermal cycled on a QuantStudio™ 12 Flex Real Time PCR System (Applied Biosystems) according to the manufacturer's instructions.

In FIG. 8, the diagonal numbers (dashed outline) represent the average Ct values of 4 replicates for the desired on-target signals. Random background noise was shown in both FIG. 8 and FIG. 9, but were usually sporadic signals detected in 1 of the 4 replicates. The background Ct values were determined to not be significant due to the large Ct differences between the on-target and off-target signals (ΔCt>10). As shown, excellent performance was observed with desirable on-target (accuracy) and non-significant off-target (specificity). The data obtained using the ATCC inclusivity panel (FIG. 8) demonstrates excellent accuracy and within-panel specificity, while the data obtained using the ATCC exclusivity panel (FIG. 9) demonstrates high specificity with closely related near neighboring species.

### Example 4

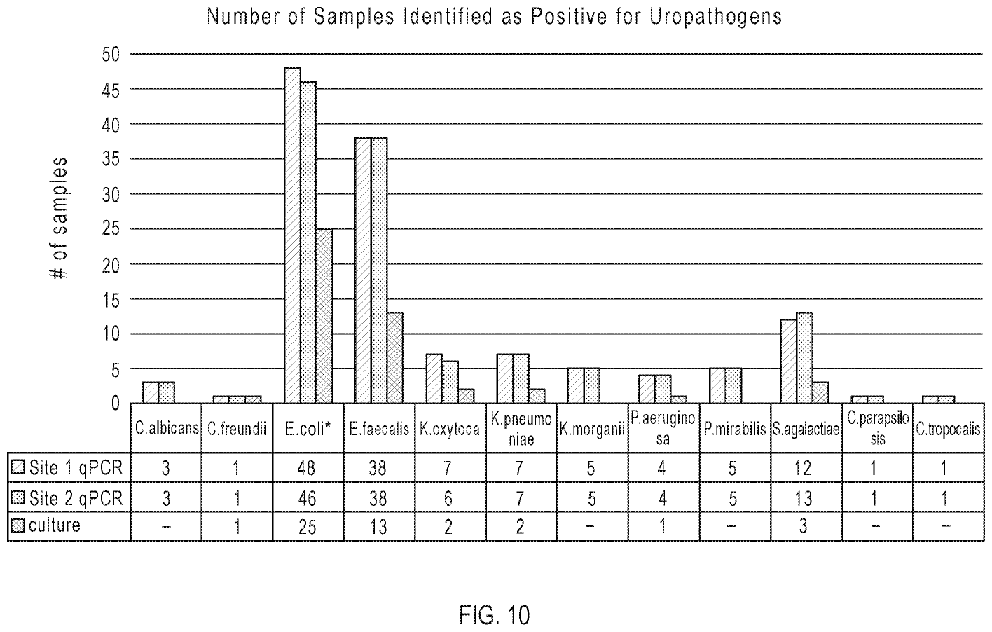

Urine repository research samples were processed using the MagMAX™ DNA Multi-Sample Ultra Kit (Thermo Fisher Scientific) on the KingFisher Flex (Thermo Fisher Scientific) platform according to the manufacturer's instructions. Samples were then screened by nanofluidic TaqMan® OpenArray® qPCR technology using target specific TaqMan® UTM assays as described above. The extracted DNA from the urine samples were run using OpenArray™ plates at two different times/locations under separate independent studies (“Site 1 qPCR” and “Site 2 qPCR”) each using 16 of the assays listed in Table 1; one assay differed between the sites (*E. Coli). The number of samples identified as positive for having the indicated uropathogens, using the UTM TaqMan assays in qPCR on OpenArray, are shown in FIG. 10 (first and second bars for each microorganism).

The urine samples were also cultured on a blood-agar plate for 24 hours and CFU/mL was counted for each sample. Uropathogens were then identified using a Vitek-2 (Biomerieux) platform according to the manufacturer's instructions. The number of urine samples identified as positive for comprising the indicated uropathogens, using the culture method, are shown in FIG. 10 (third bar for each microorganism). As FIG. 10 demonstrates, qPCR results were highly reproducible between the two separate qPCR studies performed at different locations using the UTM TaqMan assays as described herein, with >97% concordance. However, the concordance with culture compared to qPCR was lower (e.g., <80%). The data further indicates that qPCR UTM TaqMan assays were able to identify more uropathogens than traditional culture-based methods.

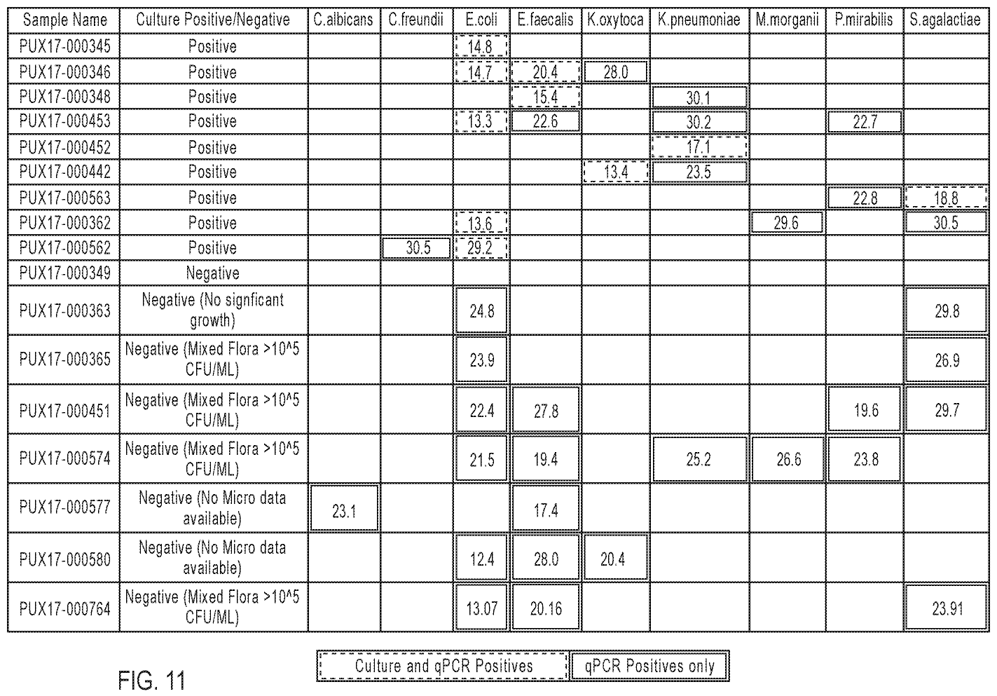

In FIG. 11, a set of urine samples was tested using the culture method and were marked as either positive or negative. Each sample having identity of at least one pathogen and showing significant growth of ≥10\textcircled{ }5 CFU/mL was marked as culture positive (See FIG. 11; second column). Culture samples were marked as culture negative if there was no significant growth (≤10\textcircled{ }5 CFU/mL) or if no micro data was available. A few additional samples were also marked as culture negative as even though they did show significant growth. This was due to culture limitations in cases where more than 2 organisms were present and it was not possible to properly distinguish the mixed flora and/or identify them as a true positive culture (versus a contaminated culture). In these cases, since more than 2 organisms were present (i.e., having a “mixed flora”), urinalysis results were found to be inconclusive or unidentifiable and categorized as culture negative. Positive and negative culture results were then compared to results obtained for the same urine samples using OpenArray™ plates using the UTM TaqMan assays as described herein and listed above in Table 1 (See FIG. 11; “Culture and qPCR Positives” vs. “qPCR Positives only”).

The results obtained from qPCR performed on OpenArray™ plates are shown as average CT values (average of three technical replicates) for various pathogens which were identified (See FIG. 11; highlighted squares). qPCR sample results which were concordant with culture results are highlighted in dark grey. qPCR results discordant with culture results are highlighted in light grey.

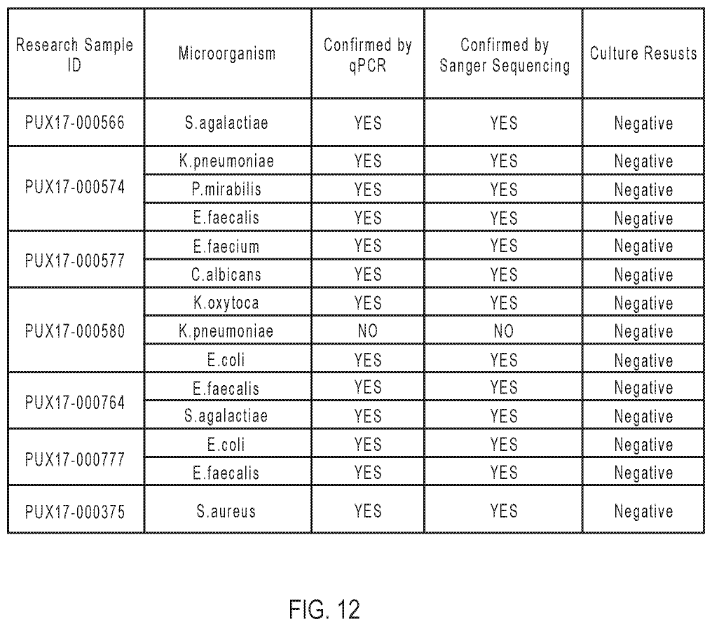

A subset of several culture discordant samples (i.e., samples which showed positive by qPCR and negative for culture growth were further verified using Sanger Sequencing methods. Sequencing results were 100% in concordance with results obtained using OpenArray™ qPCR for each sample that was tested (See FIG. 12). The results from these experiments suggest that OpenArray™ qPCR using the UTM assay panels as described herein is more sensitive than using a traditional culture method for identification and/or detection of urinary tract microbes.

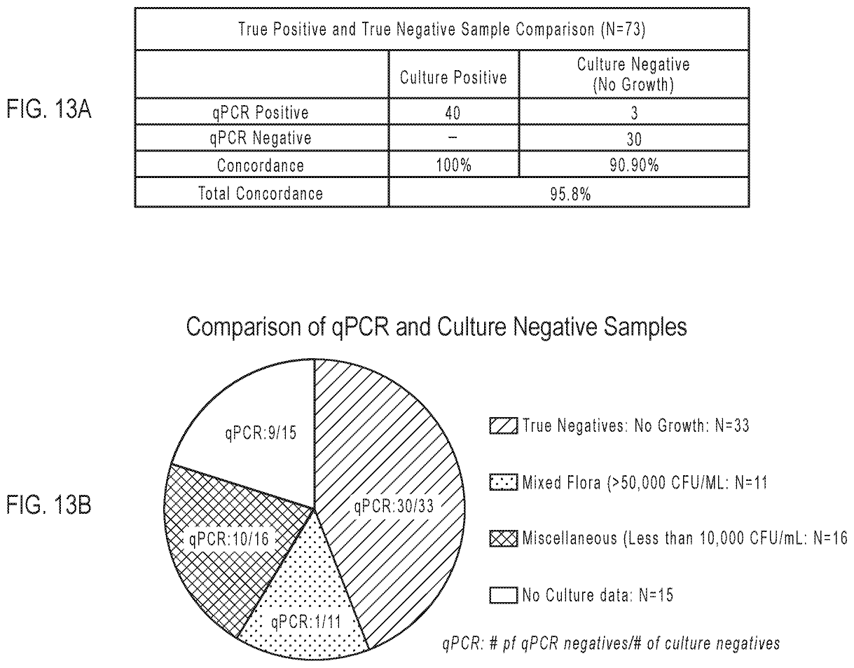

FIGS. 13A and 13B further illustrate concordance of the number of samples identified as either positive or negative for uropathogens using traditional culture methods or using the UTM TaqMan assays of Table 1. Concordance for true positives and true negatives (no growth) was 95.8%.(See FIG. 13A). All the positive samples identified by culture method were confirmed positive by qPCR assays on the OpenArray™ nanofluidic platform.

Culture negative samples were divided into different categories based on observation and limitations. qPCR using the panel of UTM TaqMan assays was able to identify more uropathogens than traditional culture methods and hence the discordance was high for total culture negative samples (See FIG. 13B).

Together this data demonstrates that OpenArray™ qPCR using the UTM TaqMan assays presented herein is more sensitive and accurate when compared to “gold standard” culture data.

1. A method for amplifying a plurality of nucleic acid sequences in a nucleic acid sample comprising:
forming at least five amplification reaction mixes each comprising an aliquot from a sample source comprising a plurality of nucleic acid sequences, using at least five different assays each comprising a pair of amplification primers, the assays selected from the group of assays in Table 1;
applying each amplification reaction mix to a reaction vessel;
performing a plurality of amplification reactions on the reaction vessel; and
detecting an amplification product corresponding to a target nucleic acid sequence within one or more locations on the reaction vessel during the plurality of amplification reactions.

2. The method of claim 1 further comprising:
utilizing the reaction in an amplification product detection system; and
operating the amplification product detection system to:
associate locations of the amplification reaction mix on the reaction vessel with one or more of the assay IDs utilized in the amplification reaction mix, optionally by use of an association table.

3. The method of claim 1, wherein the reaction vessel is a plate with a plurality of wells.

4. The method of claim 1, wherein the reaction vessel is an array.

5. The method of claim 1, wherein the reaction vessel is an open array plate.

6. The method of claim 1, wherein the reaction vessel is a chip microarray.

7. The method of claim 1, forming at least ten amplification reaction mixes each comprising an aliquot from a sample source comprising a plurality of nucleic acid sequences, using at least ten different assays selected from the group of assays in Table 1.

8. The method of claim 1, forming at least fifteen amplification reaction mixes each comprising an aliquot from a sample source comprising a plurality of nucleic acid sequences, using at least fifteen different assays selected from the group of assays in Table 1.

9. The method of claim 1, forming reaction mixes each comprising an aliquot from a sample source comprising a plurality of nucleic acid sequences, using seventeen of the assays in Table 1.

10.-54. (canceled)

55. A composition for determining the presence or absence of at least one target nucleic acid in a biological sample, the composition comprising:
at least five different amplification primer pairs, wherein each of said primers of said pairs comprise a target hybridization region that is configured to specifically hybridize to all or a portion of a region of a nucleic acid sequence of a target microorganism in Table 1 and wherein under suitable conditions said primer pair generates an amplicon; and
at least five detection probes configured to specifically hybridize to all or a portion of a region of said amplicons produced by said primer pairs.

56. The composition of claim 55, further comprising a control nucleic acid molecule comprising a plurality of different nucleic acid target sequences, said plurality of target nucleic acid sequences being specific to at least five genes in Table 1.

57. The composition of claim 55, wherein the composition is a panel or a collection of assays.

58. The composition of claim 57, wherein the panel or collection of assays comprise a panel or collection of TaqMan Assays.

59. The composition of claim 55, wherein the at least one target nucleic acid is a biomarker for a microbe associated with a urinary tract infection.

60.-265. (canceled)

266. An array for nucleic acid amplification, comprising:
a support containing a plurality of reaction sites located within the support or upon the support; each of the plurality of reaction sites containing:
(i) a control nucleic acid molecule containing a plurality of different target sequences,
(ii) an amplification primer pair configured to amplify a corresponding target sequence, and
(iii) a detectably labeled probe configured to hybridize to a nucleic acid sequence generated by extension of at least one of the amplification primers of the pair.

267. The array of claim 266, wherein at least two of the different target sequences comprise at least a 56 nucleotide portion of a gene selected from Table 1 or its corresponding cDNA.

268.-273. (canceled)

274. The array of claim 266, wherein at least one of the reaction sites includes an amplification product.

275. The array of claim 266, wherein the support includes between 10 and 10,000 reaction sites containing different amplification products.

276. The array of claim 266, wherein at least two of the reaction sites each contains a pair of amplification primers configured to amplify a different corresponding target sequence.

277.-287. (canceled)
